(19) United States
(12) Patent Application Publication
(10) Pub. No.: US 2026/0207302 A1
(43) Pub. Date: Jul. 23, 2026

(54) MULTIMODAL TIMELAPSE VISUALIZATION SYSTEM

(71) Applicant: Align Technology, Inc., San Jose, CA (US)

(72) Inventors: Michael Sabina, Campbell, CA (US); Michael Austin Brown, Orlando, FL (US); Christopher E. Cramer, Durham, NC (US)

(21) Appl. No.: 19/430,051

(22) Filed: Dec. 22, 2025

Related U.S. Application Data
(60) Provisional application No. 63/747,737, filed on Jan. 21, 2025.

(51) Int. Cl. A61C 7/00; A61B 1/24; A61C 7/08; A61C 9/00

(52) U.S. Cl. CPC A61C 7/002; A61C 7/08; A61C 9/004; A61B 1/24

(57) ABSTRACT

A method is provided for generating and/or displaying a multimodal timelapse visualization. In some cases, the method can include identifying a plurality of records associated with a dental arch of a patient. Each record corresponds to a point in time and to an imaging modality of a plurality of imaging modalities. Based on processing of at least one record, a clinical finding is determined for the patient. A primary imaging modality is determined based at least in part on the clinical finding. At least a subset of the plurality of records is normalized. A timelapse visualization representing the normalized subset of records displayed in chronological order of the plurality of points in time is generated. The timelapse visualization is provided to a user device for presentation in a user interface of the user device.

### RELATED APPLICATION

The present application claims priority to U.S. Provisional Ser. No. 63/747,737 , filed on Jan. 21, 2025, which is herein incorporated by reference in their entirety.

### TECHNICAL FIELD

The instant specification generally relates to systems and methods for generating and displaying a multimodal timelapse visualization system.

### BACKGROUND

Timelapse video techniques are used in photography and videography to convey the passage of time in a compressed and visually engaging manner. Traditional methods of creating timelapse videos typically involve capturing a series of still photographs and then stitching these images together in post production. While these methods are effective for showcasing gradual changes, they often result in videos that appear disjointed or lack smoothness. This is especially evident in timelapse videos that rely on simple fading transitions between static images, which can create a jarring or artificial effect.

Dental practitioners routinely examine and monitor oral health conditions using various imaging modalities to assess patient conditions and track changes over time. Traditional dental imaging includes intraoral and extraoral photographs, radiographic images such as bitewing and periapical X-rays, panoramic radiographs, and cone beam computed tomography (CBCT) scans. More recent technological advances have introduced intraoral optical scanning, near-infrared imaging, and other specialized imaging techniques that provide additional diagnostic information about oral tissues and structures.

Each imaging modality offers distinct advantages for visualizing different aspects of oral health. Photographic imaging provides clear visualization of surface conditions such as gingival inflammation, tooth discoloration, and aesthetic concerns. Radiographic imaging reveals subsurface conditions including caries, bone levels, and root morphology that may not be visible through clinical examination alone. Three-dimensional imaging modalities such as CBCT and intraoral scanning provide volumetric information about tooth positions, bone architecture, and spatial relationships between oral structures.

Current dental practice typically involves examining one imaging modality at a time and manually comparing images across different time points or modalities when tracking disease progression or treatment outcomes. Practitioners may review historical radiographs to assess caries progression, compare photographs to evaluate gingival changes, or analyze sequential intraoral scans to monitor tooth movement or wear patterns. This approach places substantial cognitive burden on practitioners who must mentally integrate information from multiple sources while accounting for differences in image quality, capture conditions, and temporal spacing between examinations.

Patient education and treatment planning discussions often rely on static images or hand-drawn illustrations to communicate complex oral health concepts. Practitioners may attempt to explain disease progression by showing patients individual images from different time points, but patients frequently struggle to understand subtle changes or appreciate the significance of conditions that appear minor in static representations. The abstract nature of radiographic images presents particular challenges for patient comprehension, as patients may have difficulty relating X-ray findings to their perceived oral health status.

The increasing volume and variety of dental imaging data creates additional challenges for practitioners attempting to synthesize information from multiple sources. Modern dental practices may accumulate dozens of images per patient across multiple modalities over extended time periods. Variations in image capture conditions, including lighting, patient positioning, magnification, and equipment settings, can make direct comparison between images difficult or misleading. These inconsistencies may obscure subtle but clinically significant changes or create apparent changes that are artifacts of the imaging process rather than actual biological changes.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In a first implementation, a method comprises identifying a plurality of records associated with a dental arch of a patient. Each record of the plurality of records corresponds to a point in time of a plurality of points in time and to an imaging modality of a plurality of imaging modalities. The method can further comprise determining, based on processing of at least one record of the plurality of records, a clinical finding for the patient. The method can further comprise determining a primary imaging modality of the plurality of imaging modalities based at least in part on the clinical finding. The method can further comprise normalizing at least a subset of the plurality of records. The method can further comprise generating a timelapse visualization of the dental arch of the patient. The timelapse visualization represents the normalized subset of the plurality of records displayed in chronological order of the plurality of points in time. The method can further comprise providing, to a user device, the timelapse visualization for presentation in a user interface of the user device.

A second implementation may further extend any of the first implementation. In the second implementation, the subset of the plurality of records comprises one or more of the plurality of records corresponding to the primary imaging modality.

A third implementation may further extend any of the first through second implementations. In the third implementation, the subset of the plurality of records comprises a first record corresponding to a first point in time of the plurality of points in time and to a first imaging modality of the plurality of imaging modalities, the subset of the plurality of records comprises a second record corresponding to a second point in time of the plurality of points in time and to a second imaging modality of the plurality of imaging modalities, and the timelapse visualization represents at least one of the first imaging modality or the second imaging modality.

A fourth implementation may further extend any of the first through third implementations. In the fourth implementation, the normalizing is performed across the plurality of records for at least one of a size, a scale, a color balance, a brightness, lighting conditions, contrast level, magnification factor, capture angle, or an orientation.

A fifth implementation may further extend any of the first through third implementations. In the fifth implementation, the timelapse visualization of the dental arch is presented in a dental chart.

A sixth implementation may further extend any of the first through fifth implementations. In the sixth implementation, the may may further comprise identifying a treatment corresponding to the clinical finding; generating one or more predicted records representing the dental arch of the patient affected by the treatment at a future point in time; updating the timelapse visualization of the dental arch of the patient to include the one or more predicted records; and providing, to the user device, the updated timelapse visualization for presentation in the user interface of the user device.

A seventh implementation may further extend any of the first through sixth implementations. In the seventh implementation, the treatment can include at least one of orthodontic treatment, retainer realignment treatment, restorative treatment, periodontal treatment, behavioral modification, appliance-based treatment, or interdisciplinary treatment.

An eighth implementation may further extend any of the first through seventh implementations. In the eighth implementation, the method may further comprise identifying one or more photographs of the patient. The photographs comprise at least a mouth of the patient. The method may further comprise generating one or more predicted photographs of the patient. The one or more predicted photographs represent an improvement of the at least the mouth of the mouth of the patient affected by the treatment at a corresponding future point in time. The method may further comprise generating, based on the one or more predicted photographs of the patient, a video representing the movements of the at least the mouth of the patient over time.

A ninth implementation may further extend any of the first through eighth implementations. In the ninth implementation, the one or more predicted records correspond to the primary imaging modality.

A tenth implementation may further extend any of the first through ninth implementations. In the tenth implementation, generating the one or more predicted records may further comprise determining an effect of the treatment on a condition of at least a portion of the dental arch of the patient at the future point in time, and applying the effect to a most recent record of the plurality of records.

A eleventh implementation may further extend any of the first through tenth implementations. In the eleventh implementation, the effect is determined using artificial intelligence or a predetermined set of rules.

A twelfth implementation may further extend any of the first through eleventh implementations. In the twelfth implementation, the method may further comprise generating one or more predicted records representing the dental arch of the patient affected by the clinical finding at a future point in time; updating the timelapse visualization of the dental arch of the patient to include the one or more predicted records; and providing, to the user device, the updated timelapse visualization for presentation in the user interface of the user device.

A thirteenth implementation may further extend any of the first through twelfth implementations. In the thirteenth implementation, the method may further comprise identifying one or more photographs of the patient. The photographs comprise at least a mouth of the patient. The method may further comprise generating one or more predicted photographs of the patient. The one or more predicted photographs represent a deterioration of the at least the mouth of the patient affected by the clinical finding at a corresponding future point in time. The method may further comprise generating, based on the one or more predicted photographs of the patient, a video representing the deteriorations of the at least the mouth of the patient over time.

A fourteenth implementation may further extend any of the first through thirteenth implementations. In the fourteenth implementation, generating the one or more predicted records is performed based on processing one or more of the plurality of records and sensor data corresponding to the patient. The sensor data is generated by a multisensory tool, and comprises at least one of pressure, temperature, or acceleration.

A fifteenth implementation may further extend any of the first through fourteenth implementations. In the fifteenth implementation, the one or more predicted records represent the dental arch of the patient affected by the a secondary clinical finding at the future point in time. The secondary clinical finding is associated with the clinical finding.

A sixteenth implementation may further extend any of the first through fifteenth implementations. In the sixteenth implementation, the one or more predicted records correspond to the primary imaging modality.

A seventeenth implementation may further extend any of the first through sixteenth implementations. In the seventeenth implementation, generating the one or more predicted records can comprise determining an effect of the clinical finding on a condition at least a portion of the dental arch of the patient at a future point in time, and applying the effect to a most recent record of the plurality of records.

An eighteenth implementation may further extend any of the first through seventeenth implementations. In the eighteenth implementation, the effect is determined using artificial intelligence or a predetermined set of rules.

An nineteenth implementation may further extend any of the first through eighteenth implementations. In the nineteenth implementation, the effect is determined based on at least one of patient data, familial data, general population data, or statistical analysis associated with the clinical finding.

A twentieth implementation may further extend any of the first through nineteenth implementations. In the twentieth implementation, the effect comprises a potential outcome and a velocity of progression of the clinical finding.

A twenty-first implementation may further extend any of the first through twentieth implementations. In the twenty-first implementation, determining the clinical finding for the patient comprises providing, as input, the at least one of the plurality of records to an artificial intelligence model trained to provide an indication of the clinical finding; and receiving, as output from the artificial intelligence model, the indication of the clinical finding.

A twenty-second implementation may further extend any of the first through twenty-first implementations. In the twenty-second implementation, the clinical finding comprises an indication of at least one of: caries, malocclusion, periodontal disease, tooth wear, gingival recession, tooth fracture, bruxism, temporomandibular joint disorder, structural anomaly, orthodontic relapse, or restorative disorder.

A twenty-third implementation may further extend any of the first through twenty-second implementations. In the twenty-third implementation, determining the clinical finding for the patient comprises comparing the at least one record of the plurality of records to a corresponding predetermined criterion.

A twenty-fourth implementation may further extend any of the first through twenty-third implementations. In the twenty-fourth implementation, determining the clinical finding for the patient comprises providing, as input, one or more of the plurality of records to an artificial intelligence model trained to provide an indication of the clinical finding; receiving, as output from the artificial intelligence model, a plurality of indications of the clinical finding, each indication corresponding to a record, wherein each indication comprises a confidence level; and identifying the clinical finding with a highest confidence level.

A twenty-fifth implementation may further extend any of the first through twenty-fourth implementations. In the twenty-fifth implementation, the method may further comprise providing, to the user device, each indication and the corresponding confidence level.

A twenty-sixth implementation may further extend any of the first through twenty-fifth implementations. In the twenty-sixth implementation, the primary imaging modality that corresponds to the clinical finding is determined based on a ranking of the plurality of imaging modalities.

A twenty-seventh implementation may further extend any of the first through twenty-sixth implementations. In the twenty-seventh implementation, the plurality of imaging modalities comprise at least one of intraoral scan, extraoral scan, near-infrared image, cone beam computed tomography (CBCT), photograph, video, or radiograph.

A twenty-eighth implementation may further extend any of the first through twenty-seventh implementations. In the twenty-eighth implementation, the timelapse visualization is an animated representation of the normalized subset of the plurality of records.

A twenty-ninth implementation may further extend any of the fist through twenty-eighth implementations. In the twenty-ninth implementation, generating the timelapse visualization of the dental arch comprises generating, for one or more pairs for chronologically consecutive records in the subset of the plurality of records, one or more intermediate images; and inserting, for each of the one or more pairs, the one or more intermediate images in between the corresponding pair of chronologically consecutive records.

A thirtieth implementation may further extend any of the first through twenty-ninth implementations. In the thirtieth implementation, the one or more intermediate images are generated using interpolation or optical flow techniques.

A thirty-first implementation may further extend any of the first through thirtieth implementation. In the thirty-first implementation, the timelapse visualization comprises data corresponding to at least one of the plurality of imaging modalities other than the primary imaging modality.

A thirty-second implementation may further extend any of the first through thirty-first implementations. In the thirty-second implementation, the method may further comprise identifying an area of interest in the at least one of the plurality of imaging modalities other than the primary imaging modality, wherein the area of interest corresponds to the clinical finding. The method may further comprise providing, in the timelapse visualization, a visual indicator corresponding to the area of interest in the at least one of the plurality of imaging modalities other than the primary imaging modality, wherein the visual indicator highlights the area of interest.

A thirty-third implementation may further extend any of the first through thirty-second implementation. In the thirty-third implementation, the at least one record that is processed to determine the clinical finding has a first imaging modality, and the primary imaging modality is a second imaging modality that is different from the first imaging modality.

A thirty-fourth implementation may further extend any of the first through thirty-third implementations. In the thirty-fourth implementation, the method may further comprise determining, based on the processing of the at least one record of the plurality of records, a plurality of indications of the clinical finding for the patient. Each indication of the clinical finding corresponds to an imaging modality of the plurality of imaging modalities. The method may further comprise determining a discrepancy between at least two indications of the clinical finding of the plurality of indications of the clinical finding. The method may further comprise ranking, based on a correlation between each of the at least two indications of the clinical finding and the corresponding imaging modality, each of the least two indications of the clinical finding. The method may further comprise resolving the discrepancy based on the ranking.

A thirty-fifth implementation may further extend any of the first through thirty-fourth implementations. In the thirty-fifth implementation, the method may further comprise determining a confidence for each of the least two indications of the clinical finding; determining an overall confidence by combining the confidence for each of the at least two indications of the clinical finding; and providing, the user device, the overall confidence corresponding to the clinical finding.

A thirty-sixth implementation may further extend any of the first through thirty-fifth implementations. In the thirty-sixth implementation, the method may further comprise determining, based on the processing of the at least one record of the plurality of records, a plurality of clinical findings for the patient; generating one or more predicted records representing the dental arch of the patient affected by the plurality of clinical findings at a future point in time; generating an updated timelapse visualization of the dental arch by updating the timelapse visualization to include the one or more predicted records; and providing, to the user device, the updated timelapse visualization for presentation in the user interface of the user device.

A thirty-seventh implementation may further extend any of the first through the thirty-sixth implementations. In the thirty-seventh implementation, the method may further comprise receiving, from the user device, a user interaction indicating a first clinical finding of the plurality of clinical findings; determining a subset of clinical findings comprising the plurality of clinical findings other than the first clinical finding; generating a second set of one or more predicted records representing the dental arch of the patient affected by the subset of clinical findings at the future point in time; generating a second updated timelapse visualization of the dental arch by updating the timelapse visualization to include the second set of the one or more predicted records; and providing, to the user device, the second updated timelapse visualization for presentation in the user interface of the user device.

A thirty-eighth implementation may further extend any of the first through thirty-seventh implementations. In the thirty-eighth implementation, the method may further comprise receiving, from the user device, a user interaction indicating a first clinical finding of the plurality of clinical findings; generating a third set of one or more predicted records representing the dental arch of the patient affected by the first clinical finding at the future point in time; generating a third updated timelapse visualization of the dental arch by updating the timelapse visualization to include the third set of the one or more predicted records; and providing, to the user device, the third updated timelapse visualization for presentation in the user interface of the user device.

A thirty-ninth implementation may further extend any of the first through thirty-eighth implementations. In the thirty-ninth implementation, the method may further comprise generating a first set of one or more predicted records representing the dental arch of the patient affected by the clinical finding at a future point in time; generating a second set of one or more predicted records representing the dental arch of the patient affected by behavior modifications corresponding to the clinical finding at the future point in time; generating a third set of one or more predicted records representing the dental arch of the patient affected by a first treatment corresponding to the clinical finding at the future point in time; generating a fourth set of one or more predicted records representing the dental arch of the patient affected by a second treatment corresponding to the clinical finding at the future point in time; generating a plurality of updated timelapse visualizations of the dental arch of the patient, wherein each updated timelapse visualization comprises the timelapse visualization updated to include one of the first set of one or more predicted records, the second set of one or more predicted records, the third set of one or more predicted records, or the fourth set of one or more predicted records; and providing, to the user device, the plurality of updated timelapse visualizations for presentation in the user interface of the user device.

A fortieth implementation may further extend any of the first through thirty-ninth implementations. In the fortieth implementation, the method may further comprise identifying a first record of the plurality of records corresponding to a first imaging modality of the patient at a first point in time; identifying a second record of the plurality of records corresponding to a second imaging modality of the patient at the first point in time; identifying, based on at least one of the first record or the second record, a position of one or more teeth of the dental arch and an orientation of the one or more teeth of the dental arch; and generating, based on the first record and the second record, a three-dimensional model of the dental arch and a jaw of the dental arch.

A forty-first implementation may further extend any of the first through fortieth implementations. In the forty-first implementation, the method may further comprise receiving a third record corresponding to a third imaging modality of the plurality of imaging modalities, wherein the third record corresponds to a second point in time following the first point in time. The method may further comprise identifying at least one change in the position of the one or more teeth of the dental arch or the orientation of the one or more teeth of the dental arch, wherein the at least one change corresponds to a movement of the jaw of the dental arch. The method may further comprise generating, based on the at least one change, an updated three-dimensional model of the dental arch, wherein the updated three-dimensional model represents a simulation of the movement of the jaw of the dental arch over time.

A forty-second implementation may further extend any of the first through forty-first implementations. In the forty-first implementation, the method may further comprise receiving a fourth record corresponding to a fourth imaging modality of the plurality of imaging modalities, wherein the fourth record corresponds to a second point in time following the first point in time. The method may further comprise identifying at least one change in the position of the one or more teeth of the dental arch or the orientation of the one or more teeth of the dental arch, wherein the at least one change corresponds to a movement of soft tissue of the dental arch. The method may further comprise generating, based on the at least one change, an updated three-dimensional model of the dental arch, wherein the updated three-dimensional model represents a simulation of the movement of the soft tissue of the dental arch over time.

A forty-third implementation may further extend any of the first through forty-second implementations. In the forty-third implementation, the method may further comprise determining a secondary imaging modality of the plurality of imaging modalities; normalizing at least a second subset of the plurality of records, wherein each of the second subset of the plurality of records corresponds to a subset of the plurality of points in time; generating a second timelapse visualization of the dental arch of the patient, wherein the second timelapse visualization represents the normalized second subset of the plurality of records displayed in the chronological order of the subset of the plurality of points in time; and providing, to the user device, the second timelapse visualization for presentation in the user interface of the user device.

A forty-fourth implementation may further extend any of the first through forty-third implementations. In the forty-fourth implementation, determining the secondary imaging modality can include receiving a user interaction identifying the secondary imaging modality.

A forty-fifth implementation may further extend any of the first through forty-fourth implementations. In the forty-fifth implementation, the method may further comprise causing the timelapse visualization and the second timelapse visualization to be presented concurrently in the user interface, wherein at least one point in time of the plurality of points in time of the timelapse visualization matches at least a second point in time of the subset of the plurality of points in time of the second timelapse visualization.

A forty-sixth implementation may further extend any of the first through forty-fifth implementations. In the forty-sixth implementation, the method may further comprise identifying a second imaging modality of the plurality of imaging modalities, wherein the plurality of records does not comprise a record corresponding to the second imaging modality; generating, based on the timelapse visualization, a second timelapse visualization of the dental arch of the patient, wherein the second timelapse visualization represents the timelapse visualization in the second imaging modality; and providing, to the user device, the second timelapse visualization for presentation in the user interface of the user device.

A forty-seventh implementation further extend any of the first through forty-sixth implementations. In the forty-seventh implementation, the method may further comprise identifying, based on the plurality of records associated with the dental arch of the patient, one or more events indicative of a health status of the patient, wherein each of the one or more events corresponds a particular point in time of the plurality of points in time. The method may further comprise displaying, in the timelapse visualization, a visual indicator of at least one of the one or more events, wherein the visual indicator is displayed at the particular point in time corresponding to the at least one of the one or more events.

A forty-eighth implementation further extend any of the first through forty-seventh implementations. In the forty-eighth implementation, the method may further comprise simulating, using at least one of a virtual articulator or occlusogram data of the patient, one or more forces acting on one or more teeth of the dental arch of the patient, wherein the one or more forces comprises parafunctional forces. The method may further comprise generating one or more predicted records representing the dental arch of the patient affected by the one or more forces, wherein the one or more predicted records comprises at least one of a profile view image representing a facial change of the patient. The method may comprise updating the timelapse visualization to include the one or more predicted records.

A forty-ninth implementation further extend any of the first through forty-eighth implementations. In the forty-ninth implementation, the method may further include identifying a second subset of the plurality of records, wherein the second subset comprises one or more records of the plurality of records that have a corresponding point in time after a completion of an orthodontic treatment for the patient. The method may further include detecting, based on processing of the second subset of the plurality of records, tooth movement indicating orthodontic relapse. The method may further include determining an amount of the orthodontic relapse by comparing one or more tooth positions of the second subset of the plurality of records to one or more corresponding final tooth positions associated with the orthodontic treatment for the patient. The method may further include updating the timelapse visualization to display the amount of orthodontic relapse.

A fiftieth implementation further extend any of the first through forty-ninth implementations. In the fiftieth implementation, the method may further include identifying compliance data associated with a retainer worn by the patient, wherein the compliance data comprises one or more time periods of retainer non-wear. The method may further include correlating the amount of the orthodontic relapse with the one or more time periods of retainer non-wear. The method may further include displaying, in the timelapse visualization, one or more visual indicators correlating the orthodontic relapse with the one or more time periods of retainer non-wear.

A fifty-first implementation further extend any of the first through fiftieth implementations. In the fifty-first implementation, the method may further include generating one or more predicted records representing prevention of the orthodontic relapse, wherein the timelapse visualization comprises the one or more predicted records representing prevention of orthodontic relapse.

A fifty-second implementation further extend any of the first through fifty-first implementations. In the fifty-second implementation, the method may further include responsive to determining that the amount of the orthodontic relapse satisfies a condition, generating one or more predicted records representing a correction of the orthodontic relapse. The method may further include updating the timelapse visualization to include the one or more predicted records.

In a fifty-third implementation, a system comprises a memory and a processing device to execute instructions from the memory. The processing device is configured to identify a plurality of records associated with a dental arch of a patient, wherein each record of the plurality of records corresponds to a point in time of a plurality of points in time and to an imaging modality of a plurality of imaging modalities. The processing device is further configured to determine, based on processing of at least one record of the plurality of records, a clinical finding for the patient. The processing device is further configured to determine a primary imaging modality of the plurality of the imaging modalities based at least in part on the clinical finding. The processing device is further configured to normalize at least a subset of the plurality of records. The processing device is further configured to generate a timelapse visualization of the dental arch of the patient, wherein the timelapse visualization represents the normalized subset of the plurality of records displayed in chronological order of the plurality of points in time. The processing device is further configured to provide, to a user device, the timelapse visualization for presentation in a user interface of the user device.

A fifty-fourth implementation may further extend any of the fifty-third implementation. In the fifty-fourth implementation, the subset of the plurality of records comprises one or more of the plurality of records corresponding to the primary imaging modality.

A fifty-fifth implementation may further extend any of the fifty-third through fifty-fourth implementations. In the fifty-fifth implementation, the subset of the plurality of records comprises a first record corresponding to a first point in time of the plurality of points in time and to a first imaging modality of the plurality of imaging modalities, wherein the subset of the plurality of records comprises a second record corresponding to a second point in time of the plurality of points in time and to a second imaging modality of the plurality of imaging modalities, and wherein the timelapse visualization represents at least one of the first imaging modality or the second imaging modality.

A fifty-sixth implementation may further extend any of the fifty-third through fifty-fifth implementations. In the fifty-sixth implementation, the normalizing is performed across the plurality of records for at least one of a size, a scale, a color balance, a brightness, lighting conditions, contrast level, magnification factor, capture angle, or an orientation.

A fifty-seventh implementation may further extend any of the fifty-third through fifty-sixth implementations. In the fifty-seventh implementation, the timelapse visualization of the dental arch is presented in a dental chart.

A fifty-eighth implementation may further extend any of the fifty-third through fifty-seventh implementations. In the fifty-eighth implementation, the processing device is further configured to identify a treatment corresponding to the clinical finding; generate one or more predicted records representing the dental arch of the patient affected by the treatment at a future point in time; update the timelapse visualization of the dental arch of the patient to include the one or more predicted records; and provide, to the user device, the updated timelapse visualization for presentation in the user interface of the user device.

A fifty-ninth implementation may further extend any of the fifty-third through fifty-eighth implementations. In the fifty-ninth implementation, the treatment comprises at least one of orthodontic treatment, retainer realignment treatment, restorative treatment, periodontal treatment, behavioral modification, appliance-based treatment, or interdisciplinary treatment.

A sixtieth implementation may further extend any of the fifty-third through fifty-ninth implementations. In the sixtieth implementation, the processing device is further configured to identify one or more photographs of the patient, wherein the photographs comprise at least a mouth of the patient; generate one or more predicted photographs of the patient, wherein the one or more predicted photographs represent an improvement of the at least the mouth of the patient affected by the treatment at a corresponding future point in time; and generate, based on the one or more predicted photographs of the patient, a video representing the improvements of the at least the mouth of the patient over time.

A sixty-first implementation may further extend any of the fifty-third through sixtieth implementations. In the sixty-first implementation, the one or more predicted records correspond to the primary imaging modality.

A sixty-second implementation may further extend any of the fifty-third through sixty-first implementations. In the sixty-second implementation, to generate the one or more predicted records, the processing device is further configured to determine an effect of the treatment on a condition of at least a portion of the dental arch of the patient at the future point in time; and apply the effect to a most recent record of the plurality of records.

A sixty-third implementation may further extend any of the fifty-third through sixty-second implementations. In the sixty-third implementation, the effect is determined using artificial intelligence or a predetermined set of rules.

A sixty-fourth implementation may further extend any of the fifty-third through sixty-third implementations. In the sixty-fourth implementation, the processing device is further configured to generate one or more predicted records representing the dental arch of the patient affected by the clinical finding at a future point in time; update the timelapse visualization of the dental arch of the patient to include the one or more predicted records; and provide, to the user device, the updated timelapse visualization for presentation in the user interface of the user device.

A sixty-fifth implementation may further extend any of the fifty-third through sixty-fourth implementations. In the sixty-fifth implementation, the processing device is further configured to identify one or more photographs of the patient, wherein the photographs comprise at least a mouth of the patient; generate one or more predicted photographs of the patient, wherein the one or more predicted photographs represent a deterioration of the at least the mouth of the patient affected by the clinical finding at a corresponding future point in time; and generate, based on the one or more predicted photographs of the patient, a video representing the deteriorations of the at least the mouth of the patient over time.

A sixty-sixth implementation may further extend any of the fifty-third through sixty-fifth implementations. In the sixty-sixth implementation, generating the one or more predicted records is performed based on the processing one or more of the plurality of records and sensor data corresponding to the patient, wherein the sensor data is generated by a multisensory tool, and wherein the sensor data comprises at least one of pressure, temperature, or acceleration.

A sixty-seventh implementation may further extend any of the fifty-third through sixty-sixth implementations. In the sixty-seventh implementation, the one or more predicted records represent the dental arch of the patient affected by a secondary clinical finding at the future point in time, wherein the secondary clinical finding is associated with the clinical finding.

A sixty-eighth implementation may further extend any of the fifty-third through sixty-seventh implementations. In the sixty-eighth implementation, the one or more predicted records correspond to the primary imaging modality.

A sixty-ninth implementation may further extend any of the fifty-third through sixty-eighth implementations. In the sixty-ninth implementation, to generate the one or more predicted records, the processing device is further configured to determine an effect of the clinical finding on a condition of at least a portion of the dental arch of the patient at the future point in time; and apply the effect to a most recent record of the plurality of records.

A seventieth implementation may further extend any of the fifty-third through sixty-ninth implementations. In the seventieth implementation, the effect is determined using artificial intelligence or a predetermined set of rules.

A seventy-first implementation may further extend any of the fifty-third through seventieth implementations. In the seventy-first implementation, the effect is determined based on at least one of patient data, familial data, general population data, or statistical analysis associated with the clinical finding.

A seventy-second implementation may further extend any of the fifty-third through seventy-first implementations. In the seventy-second implementation, the effect comprises a potential outcome and a velocity of progression of the clinical finding.

A seventy-third implementation may further extend any of the fifty-third through seventy-second implementations. In the seventy-third implementation, to determine the clinical finding for the patient, the processing device is further configured to provide, as input, the at least one of the plurality of records to an artificial intelligence model trained to provide an indication of the clinical finding; and receive, as output from the artificial intelligence model, the indication of the clinical finding.

A seventy-fourth implementation may further extend any of the fifty-third through seventy-third implementations. In the seventy-fourth implementation, the clinical finding comprises an indication of at least one of: caries, malocclusion, periodontal disease, tooth wear, gingival recession, tooth fracture, bruxism, temporomandibular joint disorder, structural anomaly, orthodontic relapse, or restorative disorder.

A seventy-fifth implementation may further extend any of the fifty-third through seventy-fourth implementations. In the seventy-fifth implementation, to determine the clinical finding for the patient, the processing device is further configured to compare the at least one record of the plurality of records to a corresponding predetermined criterion.

A seventy-sixth implementation may further extend any of the fifty-third through seventy-fifth implementations. In the seventy-sixth implementation, to determine the clinical finding for the patient, the processing device is further configured to provide, as input, one or more of the plurality of records to an artificial intelligence model trained to provide an indication of the clinical finding; receive, as output from the artificial intelligence model, a plurality of indications of the clinical finding, each indication corresponding to a record, wherein each indication comprises a confidence level; and identify the clinical finding with a highest confidence level.

A seventy-seventh implementation may further extend any of the fifty-third through seventy-sixth implementations. In the seventy-seventh implementation, the processing device is further configured to provide, to the user device, each indication and the corresponding confidence level.

A seventy-eighth implementation may further extend any of the fifty-third through seventy-seventh implementations. In the seventy-eighth implementation, the primary imaging modality that corresponds to the clinical finding is determined based on a ranking of the plurality of imaging modalities.

A seventy-ninth implementation may further extend any of the fifty-third through seventy-eighth implementations. In the seventy-ninth implementation, the plurality of imaging modalities comprise at least one of: intraoral scan, extraoral scan, near-infrared image, cone beam computed tomography (CBCT), photograph, video, or radiograph.

An eightieth implementation may further extend any of the fifty-third through seventy-ninth implementations. In the eightieth implementation, the timelapse visualization is an animated representation of the normalized subset of the plurality of records.

An eighty-first implementation may further extend any of the fifty-third through eightieth implementations. In the eighty-first implementation, to generate the timelapse visualization of the dental arch, the processing device is further configured to generate, for one or more pairs of chronologically consecutive records in the subset of the plurality of records, one or more intermediate images; and insert, for each of the one or more pairs, the one or more intermediate images in between the corresponding pair of chronologically consecutive records.

An eighty-second implementation may further extend any of the fifty-third through eighty-first implementations. In the eighty-second implementation, the one or more intermediate images are generated using interpolation or optical flow techniques.

An eighty-third implementation may further extend any of the fifty-third through eighty-second implementations. In the eighty-third implementation, the timelapse visualization comprises data corresponding to at least one of the plurality of imaging modalities other than the primary imaging modality.

An eighty-fourth implementation may further extend any of the fifty-third through eighty-third implementations. In the eighty-fourth implementation, the processing device is further configured to identify an area of interest in the at least one of the plurality of imaging modalities other than the primary imaging modality, wherein the area of interest corresponds to the clinical finding; and provide, in the timelapse visualization, a visual indicator corresponding to the area of interest in the at least one of the plurality of imaging modalities other than the primary imaging modality, wherein the visual indicator highlights the area of interest.

An eighty-fifth implementation may further extend any of the fifty-third through eighty-fourth implementations. In the eighty-fifth implementation, the at least one record that is processed to determine the clinical finding has a first imaging modality, and wherein the primary imaging modality is a second imaging modality that is different from the first imaging modality.

An eighty-sixth implementation may further extend any of the fifty-third through eighty-fifth implementations. In the eighty-sixth implementation, the processing device is further configured to determine, based on the processing of the at least one record of the plurality of records, a plurality of indications of the clinical finding for the patient, wherein each indication of the clinical finding corresponds to an imaging modality of the plurality of imaging modalities; determine a discrepancy between at least two indications of the clinical finding of the plurality of indications of the clinical finding; rank, based on a correlation between each of the at least two indications of clinical finding and the corresponding imaging modality, each of the at least two indications of the clinical finding; and resolve the discrepancy based on the ranking.

An eighty-seventh implementation may further extend any of the fifty-third through eighty-sixth implementations. In the eighty-seventh implementation, the processing device is further configured to determine a confidence for each of the at least two indications of the clinical finding; determine an overall confidence by combining the confidence for each of the at least two indications of the clinical finding; and provide, to the user device, the overall confidence corresponding to the clinical finding.

An eighty-eighth implementation may further extend any of the fifty-third through eighty-seventh implementations. In the eighty-eighth implementation, the processing device is further configured to determine, based on the processing of the at least one record of the plurality of records, a plurality of clinical findings for the patient; generate one or more predicted records representing the dental arch of the patient affected by the plurality of clinical findings at a future point in time; generate an updated timelapse visualization of the dental arch by updating the timelapse visualization to include the one or more predicted records; and provide, to the user device, the updated timelapse visualization for presentation in the user interface of the user device.

An eighty-ninth implementation may further extend any of the fifty-third through eighty-eighth implementations. In the eighty-ninth implementation, the processing device is further configured to receive, from the user device, a user interaction indicating a first clinical finding of the plurality of clinical findings; determine a subset of clinical findings comprising the plurality of clinical findings other than first clinical finding; generate a second set of one or more predicted records representing the dental arch of the patient affected by the subset of clinical findings at the future point in time; generate a second updated timelapse visualization of the dental arch by updating the timelapse visualization to include the second set of the one or more predicted records; and provide, to the user device, the second updated timelapse visualization for presentation in the user interface of the user device.

A ninetieth implementation may further extend any of the fifty-third through eighty-ninth implementations. In the ninetieth implementation, the processing device is further configured to receive, from the user device, a user interaction indicating a first clinical finding of the plurality of clinical findings; generate a third set of one or more predicted records representing the dental arch of the patient affected by the first clinical finding at the future point in time; generate a third updated timelapse visualization of the dental arch by updating the timelapse visualization to include the third set of the one or more predicted records; and provide, to the user device, the third updated timelapse visualization for presentation in the user interface of the user device.

A ninety-first implementation may further extend any of the fifty-third through ninetieth implementations. In the ninety-first implementation, the processing device is further configured to generate a first set of one or more predicted records representing the dental arch of the patient affected by the clinical finding at a future point in time; generate a second set of one or more predicted records representing the dental arch of the patient affected by behavior modifications corresponding to the clinical finding at the future point in time; generate a third set of one or more predicted records representing the dental arch of the patient affected by a first treatment corresponding to the clinical finding at the future point in time; generate a fourth set of one or more predicted records representing the dental arch of the patient affected by a second treatment corresponding to the clinical finding at the future point in time; generate a plurality of updated timelapse visualizations of the dental arch of the patient, wherein each updated timelapse visualization comprises the timelapse visualization updated to include one of the first set of one or more predicted records, the second set of one or more predicted records, the third set of one or more predicted records, or the fourth set of one or more predicted records; and provide, to the user device, the plurality of updated timelapse visualizations for presentation in the user interface of the user device.

A ninety-second implementation may further extend any of the fifty-third through ninety-first implementations. In the ninety-second implementation, the processing device is further configured to identify a first record of the plurality of records corresponding to a first imaging modality of the patient at a first point in time; identify a second record of the plurality of records corresponding to a second imaging modality of the dental arch of the patient at the first point in time; identify, based on at least one of the first record or the second record, a position of one or more teeth of the dental arch and an orientation of the one or more teeth of the dental arch; and generate, based on the first record and the second record, a three-dimensional model of the dental arch of the patient, wherein the three-dimensional model comprises the one or more teeth of the dental arch and a jaw of the dental arch.

A ninety-third implementation may further extend any of the fifty-third through ninety-second implementations. In the ninety-third implementation, the processing device is further configured to receive a third record corresponding to a third imaging modality of the plurality of imaging modalities, wherein the third record corresponds to a second point in time following the first point in time; identify at least one change in the position of the one or more teeth of the dental arch or the orientation of the one or more teeth of the dental arch, wherein the at least one change corresponds to a movement of the jaw of the dental arch; and generate, based on the at least one change, an updated three-dimensional model of the dental arch, wherein the updated three-dimensional model represents a simulation of the movement of the jaw of the dental arch over time.

A ninety-fourth implementation may further extend any of the fifty-third through ninety-third implementations. In the ninety-fourth implementation, the processing device is further configured to receive a fourth record corresponding to a fourth imaging modality of the plurality of imaging modalities, wherein the fourth record corresponds to a second point in time following the first point in time; identify at least one change in the position of the one or more teeth of the dental arch or the orientation of the one or more teeth of the dental arch, wherein the at least one change corresponds to a movement of soft tissue of the dental arch; and generate, based on the at least one change, an updated three-dimensional model of the dental arch, wherein the updated three-dimensional model represents a simulation of the movement of the soft tissue of the dental arch over time.

A ninety-fifth implementation may further extend any of the fifty-third through ninety-fourth implementations. In the ninety-fifth implementation, the processing device is further configured to determine a secondary imaging modality of the plurality of imaging modalities; normalize at least a second subset of the plurality of records, wherein each record of the second subset of the plurality of records corresponds to a subset of the plurality of points in time; generate a second timelapse visualization of the dental arch of the patient, wherein the second timelapse visualization represents the normalized second subset of the plurality of records displayed in the chronological order of the subset of the plurality of points in time; and provide, to the user device, the second timelapse visualization for presentation in the user interface of the user device.

A ninety-sixth implementation may further extend any of the fifty-third through ninety-fifth implementations. In the ninety-sixth implementation, to determine the secondary imaging modality, the processing device is further configured to receive a user interaction identifying the secondary imaging modality.

A ninety-seventh implementation may further extend any of the fifty-third through ninety-sixth implementations. In the ninety-seventh implementation, the processing device is further configured to cause the timelapse visualization and the second timelapse visualization to be presented concurrently in the user interface, wherein at least one point in time of the plurality of points in time of the timelapse visualization matches at least a second point in time of the subset of the plurality of points in time of the second timelapse visualization.

A ninety-eighth implementation may further extend any of the fifty-third through ninety-seventh implementations. In the ninety-eighth implementation, the processing device is further configured to identify a second imaging modality of the plurality of imaging modalities, wherein the plurality of records does not comprise a record corresponding to the second imaging modality; generate, based on the timelapse visualization, a second timelapse visualization of the dental arch of the patient, wherein the second timelapse visualization represents the timelapse visualization in the second imaging modality; and provide, to the user device, the second timelapse visualization for presentation in the user interface of the user device.

A ninety-ninth implementation may further extend any of the fifty-third through ninety-eighth implementations. In the ninety-ninth implementation, the processing device is further configured to identify, based on the plurality of records associated with the dental arch of the patient, one or more events indicative of a health status of the patient, wherein each of the one or more events corresponds a particular point in time of the plurality of points in time; and display, in the timelapse visualization, a visual indicator of at least one of the one or more events, wherein the visual indicator is displayed at the particular point in time corresponding to the at least one of the one or more events.

A one hundredth implementation may further extend any of the fifty-third through ninety-ninth implementations. In the one hundredth implementation, the processing device is further configured to simulate, using at least one of a virtual articulator or occlusogram data of the patient, one or more forces acting on one or more teeth of the dental arch of the patient, wherein the one or more forces comprises parafunctional forces; generate one or more predicted records representing the dental arch of the patient affected by the one or more forces, wherein the one or more predicted records comprises at least one of a profile view image representing a facial change of the patient; and update the timelapse visualization to include the one or more predicted records.

A one hundred first implementation may further extend any of the fifty-third through one hundredth implementations. In the one hundred first implementation, the processing device is further configured to identify a second subset of the plurality of records, wherein the second subset comprises one or more records of the plurality of records that have a corresponding point in time after a completion of an orthodontic treatment for the patient; detect, based on processing of the second subset of the plurality of records, tooth movement indicating orthodontic relapse; determine an amount of the orthodontic relapse by comparing one or more tooth positions of the second subset of the plurality of records to one or more corresponding final tooth positions associated with the orthodontic treatment for the patient; and update the timelapse visualization to display the amount of orthodontic relapse.

A one hundred second implementation may further extend any of the fifty-third through one hundred first implementations. In the one hundred second implementation, the processing device is further configured to identify compliance data associated with a retainer worn by the patient, wherein the compliance data comprises one or more time periods of retainer non-wear; correlate the amount of the orthodontic relapse with the one or more time periods of retainer non-wear; and display, in the timelapse visualization, one or more visual indicators correlating the orthodontic relapse with the one or more time periods of retainer non-wear.

A one hundred third implementation may further extend any of the fifty-third through one hundred second implementations. In the one hundred third implementation, the processing device is further configured to generate one or more predicted records representing prevention of the orthodontic relapse, wherein the timelapse visualization comprises the one or more predicted records representing prevention of orthodontic relapse.

A one hundred fourth implementation may further extend any of the fifty-third through one hundred third implementations. In the one hundred fourth implementation, the processing device is further configured to responsive to determining that the amount of the orthodontic relapse satisfies a condition, generate one or more predicted records representing a correction of the orthodontic relapse; and update the timelapse visualization to include the one or more predicted records.

In a one hundred fifth implementation, a non-transitory computer-readable storage medium comprises instructions that, when executed by a processing device, cause the processing device to identify a plurality of records associated with a dental arch of a patient, wherein each record of the plurality of records corresponds to a point in time of a plurality of points in time and to an imaging modality of a plurality of imaging modalities. The instructions further cause the processing device to determine, based on processing of at least one record of the plurality of records, a clinical finding for the patient. The instructions further cause the processing device to determine a primary imaging modality of the plurality of the imaging modalities based at least in part on the clinical finding. The instructions further cause the processing device to normalize at least a subset of the plurality of records. The instructions further cause the processing device to generate a timelapse visualization of the dental arch of the patient, wherein the timelapse visualization represents the normalized subset of the plurality of records displayed in chronological order of the plurality of points in time. The instructions further cause the processing device to provide, to a user device, the timelapse visualization for presentation in a user interface of the user device.

A one hundred sixth implementation may further extend any of the one hundred fifth implementation. In the one hundred sixth implementation, the subset of the plurality of records comprises one or more of the plurality of records corresponding to the primary imaging modality.

A one hundred seventh implementation may further extend any of the one hundred fifth through one hundred sixth implementations. In the one hundred seventh implementation, the subset of the plurality of records comprises a first record corresponding to a first point in time of the plurality of points in time and to a first imaging modality of the plurality of imaging modalities, wherein the subset of the plurality of records comprises a second record corresponding to a second point in time of the plurality of points in time and to a second imaging modality of the plurality of imaging modalities, and wherein the timelapse visualization represents at least one of the first imaging modality or the second imaging modality.

A one hundred eighth implementation may further extend any of the one hundred fifth through one hundred seventh implementations. In the one hundred eighth implementation, the normalizing is performed across the plurality of records for at least one of a size, a scale, a color balance, a brightness, lighting conditions, contrast level, magnification factor, capture angle, or an orientation.

A one hundred ninth implementation may further extend any of the one hundred fifth through one hundred eighth implementations. In the one hundred ninth implementation, the timelapse visualization of the dental arch is presented in a dental chart.

A one hundred tenth implementation may further extend any of the one hundred fifth through one hundred ninth implementations. In the one hundred tenth implementation, the processing device is further to identify a treatment corresponding to the clinical finding; generate one or more predicted records representing the dental arch of the patient affected by the treatment at a future point in time; update the timelapse visualization of the dental arch of the patient to include the one or more predicted records; and provide, to the user device, the updated timelapse visualization for presentation in the user interface of the user device.

A one hundred eleventh implementation may further extend any of the one hundred fifth through one hundred tenth implementations. In the one hundred eleventh implementation, the treatment comprises at least one of orthodontic treatment, retainer realignment treatment, restorative treatment, periodontal treatment, behavioral modification, appliance-based treatment, or interdisciplinary treatment.

A one hundred twelfth implementation may further extend any of the one hundred fifth through one hundred eleventh implementations. In the one hundred twelfth implementation, the processing device is further to identify one or more photographs of the patient, wherein the photographs comprise at least a mouth of the patient; generate one or more predicted photographs of the patient, wherein the one or more predicted photographs represent an improvement of the at least the mouth of the patient affected by the treatment at a corresponding future point in time; and generate, based on the one or more predicted photographs of the patient, a video representing the improvements of the at least the mouth of the patient over time.

A one hundred thirteenth implementation may further extend any of the one hundred fifth through one hundred twelfth implementations. In the one hundred thirteenth implementation, the one or more predicted records correspond to the primary imaging modality.

A one hundred fourteenth implementation may further extend any of the one hundred fifth through one hundred thirteenth implementations. In the one hundred fourteenth implementation, to generate the one or more predicted records, the processing device is further to determine an effect of the treatment on a condition of at least a portion of the dental arch of the patient at the future point in time; and apply the effect to a most recent record of the plurality of records.

A one hundred fifteenth implementation may further extend any of the one hundred fifth through one hundred fourteenth implementations. In the one hundred fifteenth implementation, the effect is determined using artificial intelligence or a predetermined set of rules.

A one hundred sixteenth implementation may further extend any of the one hundred fifth through one hundred fifteenth implementations. In the one hundred sixteenth implementation, the processing device is further to generate one or more predicted records representing the dental arch of the patient affected by the clinical finding at a future point in time; update the timelapse visualization of the dental arch of the patient to include the one or more predicted records; and provide, to the user device, the updated timelapse visualization for presentation in the user interface of the user device.

A one hundred seventeenth implementation may further extend any of the one hundred fifth through one hundred sixteenth implementations. In the one hundred seventeenth implementation, the processing device is further to identify one or more photographs of the patient, wherein the photographs comprise at least a mouth of the patient; generate one or more predicted photographs of the patient, wherein the one or more predicted photographs represent a deterioration of the at least the mouth of the patient affected by the clinical finding at a corresponding future point in time; and generate, based on the one or more predicted photographs of the patient, a video representing the deteriorations of the at least the mouth of the patient over time.

A one hundred eighteenth implementation may further extend any of the one hundred fifth through one hundred seventeenth implementations. In the one hundred eighteenth implementation, generating the one or more predicted records is performed based on the processing one or more of the plurality of records and sensor data corresponding to the patient, wherein the sensor data is generated by a multisensory tool, and wherein the sensor data comprises at least one of pressure, temperature, or acceleration.

A one hundred nineteenth implementation may further extend any of the one hundred fifth through one hundred eighteenth implementations. In the one hundred nineteenth implementation, the one or more predicted records represent the dental arch of the patient affected by a secondary clinical finding at the future point in time, wherein the secondary clinical finding is associated with the clinical finding.

A one hundred twentieth implementation may further extend any of the one hundred fifth through one hundred nineteenth implementations. In the one hundred twentieth implementation, the one or more predicted records correspond to the primary imaging modality.

A one hundred twenty-first implementation may further extend any of the one hundred fifth through one hundred twentieth implementations. In the one hundred twenty-first implementation, to generate the one or more predicted records, the processing device is further to determine an effect of the clinical finding on a condition of at least a portion of the dental arch of the patient at the future point in time; and apply the effect to a most recent record of the plurality of records.

A one hundred twenty-second implementation may further extend any of the one hundred fifth through one hundred twenty-first implementations. In the one hundred twenty-second implementation, the effect is determined using artificial intelligence or a predetermined set of rules.

A one hundred twenty-third implementation may further extend any of the one hundred fifth through one hundred twenty-second implementations. In the one hundred twenty-third implementation, the effect is determined based on at least one of patient data, familial data, general population data, or statistical analysis associated with the clinical finding.

A one hundred twenty-fourth implementation may further extend any of the one hundred fifth through one hundred twenty-third implementations. In the one hundred twenty-fourth implementation, the effect comprises a potential outcome and a velocity of progression of the clinical finding.

A one hundred twenty-fifth implementation may further extend any of the one hundred fifth through one hundred twenty-fourth implementations. In the one hundred twenty-fifth implementation, to determine the clinical finding for the patient, the processing device is further to provide, as input, the at least one of the plurality of records to an artificial intelligence model trained to provide an indication of the clinical finding; and receive, as output from the artificial intelligence model, the indication of the clinical finding.

A one hundred twenty-sixth implementation may further extend any of the one hundred fifth through one hundred twenty-fifth implementations. In the one hundred twenty-sixth implementation, the clinical finding comprises an indication of at least one of: caries, malocclusion, periodontal disease, tooth wear, gingival recession, tooth fracture, bruxism, temporomandibular joint disorder, structural anomaly, orthodontic relapse, or restorative disorder.

A one hundred twenty-seventh implementation may further extend any of the one hundred fifth through one hundred twenty-sixth implementations. In the one hundred twenty-seventh implementation, to determine the clinical finding for the patient, the processing device is further to compare the at least one record of the plurality of records to a corresponding predetermined criterion.

A one hundred twenty-eighth implementation may further extend any of the one hundred fifth through one hundred twenty-seventh implementations. In the one hundred twenty-eighth implementation, to determine the clinical finding for the patient, the processing device is further to provide, as input, one or more of the plurality of records to an artificial intelligence model trained to provide an indication of the clinical finding; receive, as output from the artificial intelligence model, a plurality of indications of the clinical finding, each indication corresponding to a record, wherein each indication comprises a confidence level; and identify the clinical finding with a highest confidence level.

A one hundred twenty-ninth implementation may further extend any of the one hundred fifth through one hundred twenty-eighth implementations. In the one hundred twenty-ninth implementation, the processing device is further to provide, to the user device, each indication and the corresponding confidence level.

A one hundred thirtieth implementation may further extend any of the one hundred fifth through one hundred twenty-ninth implementations. In the one hundred thirtieth implementation, the primary imaging modality that corresponds to the clinical finding is determined based on a ranking of the plurality of imaging modalities.

A one hundred thirty-first implementation may further extend any of the one hundred fifth through one hundred thirtieth implementations. In the one hundred thirty-first implementation, the plurality of imaging modalities comprise at least one of: intraoral scan, extraoral scan, near-infrared image, cone beam computed tomography (CBCT), photograph, video, or radiograph.

A one hundred thirty-second implementation may further extend any of the one hundred fifth through one hundred thirty-first implementations. In the one hundred thirty-second implementation, the timelapse visualization is an animated representation of the normalized subset of the plurality of records.

A one hundred thirty-third implementation may further extend any of the one hundred fifth through one hundred thirty-second implementations. In the one hundred thirty-third implementation, to generate the timelapse visualization of the dental arch, the processing device is further to generate, for one or more pairs of chronologically consecutive records in the subset of the plurality of records, one or more intermediate images; and insert, for each of the one or more pairs, the one or more intermediate images in between the corresponding pair of chronologically consecutive records.

A one hundred thirty-fourth implementation may further extend any of the one hundred fifth through one hundred thirty-third implementations. In the one hundred thirty-fourth implementation, the one or more intermediate images are generated using interpolation or optical flow techniques.

A one hundred thirty-fifth implementation may further extend any of the one hundred fifth through one hundred thirty-fourth implementations. In the one hundred thirty-fifth implementation, the timelapse visualization comprises data corresponding to at least one of the plurality of imaging modalities other than the primary imaging modality.

A one hundred thirty-sixth implementation may further extend any of the one hundred fifth through one hundred thirty-fifth implementations. In the one hundred thirty-sixth implementation, the processing device is further to identify an area of interest in the at least one of the plurality of imaging modalities other than the primary imaging modality, wherein the area of interest corresponds to the clinical finding; and provide, in the timelapse visualization, a visual indicator corresponding to the area of interest in the at least one of the plurality of imaging modalities other than the primary imaging modality, wherein the visual indicator highlights the area of interest.

A one hundred thirty-seventh implementation may further extend any of the one hundred fifth through one hundred thirty-sixth implementations. In the one hundred thirty-seventh implementation, the at least one record that is processed to determine the clinical finding has a first imaging modality, and wherein the primary imaging modality is a second imaging modality that is different from the first imaging modality.

A one hundred thirty-eighth implementation may further extend any of the one hundred fifth through one hundred thirty-seventh implementations. In the one hundred thirty-eighth implementation, the processing device is further to determine, based on the processing of the at least one record of the plurality of records, a plurality of indications of the clinical finding for the patient, wherein each indication of the clinical finding corresponds to an imaging modality of the plurality of imaging modalities; determine a discrepancy between at least two indications of the clinical finding of the plurality of indications of the clinical finding; rank, based on a correlation between each of the at least two indications of clinical finding and the corresponding imaging modality, each of the at least two indications of the clinical finding; and resolve the discrepancy based on the ranking.

A one hundred thirty-ninth implementation may further extend any of the one hundred fifth through one hundred thirty-eighth implementations. In the one hundred thirty-ninth implementation, the processing device is further to determine a confidence for each of the at least two indications of the clinical finding; determine an overall confidence by combining the confidence for each of the at least two indications of the clinical finding; and provide, to the user device, the overall confidence corresponding to the clinical finding.

A one hundred fortieth implementation may further extend any of the one hundred fifth through one hundred thirty-ninth implementations. In the one hundred fortieth implementation, the processing device is further to determine, based on the processing of the at least one record of the plurality of records, a plurality of clinical findings for the patient; generate one or more predicted records representing the dental arch of the patient affected by the plurality of clinical findings at a future point in time; generate an updated timelapse visualization of the dental arch by updating the timelapse visualization to include the one or more predicted records; and provide, to the user device, the updated timelapse visualization for presentation in the user interface of the user device.

A one hundred forty-first implementation may further extend any of the one hundred fifth through one hundred fortieth implementations. In the one hundred forty-first implementation, the processing device is further to receive, from the user device, a user interaction indicating a first clinical finding of the plurality of clinical findings; determine a subset of clinical findings comprising the plurality of clinical findings other than first clinical finding; generate a second set of one or more predicted records representing the dental arch of the patient affected by the subset of clinical findings at the future point in time; generate a second updated timelapse visualization of the dental arch by updating the timelapse visualization to include the second set of the one or more predicted records; and provide, to the user device, the second updated timelapse visualization for presentation in the user interface of the user device.

A one hundred forty-second implementation may further extend any of the one hundred fifth through one hundred forty-first implementations. In the one hundred forty-second implementation, the processing device is further to receive, from the user device, a user interaction indicating a first clinical finding of the plurality of clinical findings; generate a third set of one or more predicted records representing the dental arch of the patient affected by the first clinical finding at the future point in time; generate a third updated timelapse visualization of the dental arch by updating the timelapse visualization to include the third set of the one or more predicted records; and provide, to the user device, the third updated timelapse visualization for presentation in the user interface of the user device.

A one hundred forty-third implementation may further extend any of the one hundred fifth through one hundred forty-second implementations. In the one hundred forty-third implementation, the processing device is further to generate a first set of one or more predicted records representing the dental arch of the patient affected by the clinical finding at a future point in time; generate a second set of one or more predicted records representing the dental arch of the patient affected by behavior modifications corresponding to the clinical finding at the future point in time; generate a third set of one or more predicted records representing the dental arch of the patient affected by a first treatment corresponding to the clinical finding at the future point in time; generate a fourth set of one or more predicted records representing the dental arch of the patient affected by a second treatment corresponding to the clinical finding at the future point in time; generate a plurality of updated timelapse visualizations of the dental arch of the patient, wherein each updated timelapse visualization comprises the timelapse visualization updated to include one of the first set of one or more predicted records, the second set of one or more predicted records, the third set of one or more predicted records, or the fourth set of one or more predicted records; and provide, to the user device, the plurality of updated timelapse visualizations for presentation in the user interface of the user device.

A one hundred forty-fourth implementation may further extend any of the one hundred fifth through one hundred forty-third implementations. In the one hundred forty-fourth implementation, the processing device is further to identify a first record of the plurality of records corresponding to a first imaging modality of the patient at a first point in time; identify a second record of the plurality of records corresponding to a second imaging modality of the dental arch of the patient at the first point in time; identify, based on at least one of the first record or the second record, a position of one or more teeth of the dental arch and an orientation of the one or more teeth of the dental arch; and generate, based on the first record and the second record, a three-dimensional model of the dental arch of the patient, wherein the three-dimensional model comprises the one or more teeth of the dental arch and a jaw of the dental arch.

A one hundred forty-fifth implementation may further extend any of the one hundred fifth through one hundred forty-fourth implementations. In the one hundred forty-fifth implementation, the processing device is further to receive a third record corresponding to a third imaging modality of the plurality of imaging modalities, wherein the third record corresponds to a second point in time following the first point in time; identify at least one change in the position of the one or more teeth of the dental arch or the orientation of the one or more teeth of the dental arch, wherein the at least one change corresponds to a movement of the jaw of the dental arch; and generate, based on the at least one change, an updated three-dimensional model of the dental arch, wherein the updated three-dimensional model represents a simulation of the movement of the jaw of the dental arch over time.

A one hundred forty-sixth implementation may further extend any of the one hundred fifth through one hundred forty-fifth implementations. In the one hundred forty-sixth implementation, the processing device is further to receive a fourth record corresponding to a fourth imaging modality of the plurality of imaging modalities, wherein the fourth record corresponds to a second point in time following the first point in time; identify at least one change in the position of the one or more teeth of the dental arch or the orientation of the one or more teeth of the dental arch, wherein the at least one change corresponds to a movement of soft tissue of the dental arch; and generate, based on the at least one change, an updated three-dimensional model of the dental arch, wherein the updated three-dimensional model represents a simulation of the movement of the soft tissue of the dental arch over time.

A one hundred forty-seventh implementation may further extend any of the one hundred fifth through one hundred forty-sixth implementations. In the one hundred forty-seventh implementation, the processing device is further to determine a secondary imaging modality of the plurality of imaging modalities; normalize at least a second subset of the plurality of records, wherein each record of the second subset of the plurality of records corresponds to a subset of the plurality of points in time; generate a second timelapse visualization of the dental arch of the patient, wherein the second timelapse visualization represents the normalized second subset of the plurality of records displayed in the chronological order of the subset of the plurality of points in time; and provide, to the user device, the second timelapse visualization for presentation in the user interface of the user device.

A one hundred forty-eighth implementation may further extend any of the one hundred fifth through one hundred forty-seventh implementations. In the one hundred forty-eighth implementation, to determine the secondary imaging modality, the processing device is further to receive a user interaction identifying the secondary imaging modality.

A one hundred forty-ninth implementation may further extend any of the one hundred fifth through one hundred forty-eighth implementations. In the one hundred forty-ninth implementation, the processing device is further to cause the timelapse visualization and the second timelapse visualization to be presented concurrently in the user interface, wherein at least one point in time of the plurality of points in time of the timelapse visualization matches at least a second point in time of the subset of the plurality of points in time of the second timelapse visualization.

A one hundred fiftieth implementation may further extend any of the one hundred fifth through one hundred forty-ninth implementations. In the one hundred fiftieth implementation, the processing device is further to identify a second imaging modality of the plurality of imaging modalities, wherein the plurality of records does not comprise a record corresponding to the second imaging modality; generate, based on the timelapse visualization, a second timelapse visualization of the dental arch of the patient, wherein the second timelapse visualization represents the timelapse visualization in the second imaging modality; and provide, to the user device, the second timelapse visualization for presentation in the user interface of the user device.

A one hundred fifty-first implementation may further extend any of the one hundred fifth through one hundred fiftieth implementations. In the one hundred fifty-first implementation, the processing device is further to identify, based on the plurality of records associated with the dental arch of the patient, one or more events indicative of a health status of the patient, wherein each of the one or more events corresponds a particular point in time of the plurality of points in time; and display, in the timelapse visualization, a visual indicator of at least one of the one or more events, wherein the visual indicator is displayed at the particular point in time corresponding to the at least one of the one or more events.

A one hundred fifty-second implementation may further extend any of the one hundred fifth through one hundred fifty-first implementations. In the one hundred fifty-second implementation, the processing device is further to simulate, using at least one of a virtual articulator or occlusogram data of the patient, one or more forces acting on one or more teeth of the dental arch of the patient, wherein the one or more forces comprises parafunctional forces; generate one or more predicted records representing the dental arch of the patient affected by the one or more forces, wherein the one or more predicted records comprises at least one of a profile view image representing a facial change of the patient; and update the timelapse visualization to include the one or more predicted records.

A one hundred fifty-third implementation may further extend any of the one hundred fifth through one hundred fifty-second implementations. In the one hundred fifty-third implementation, the processing device is further to identify a second subset of the plurality of records, wherein the second subset comprises one or more records of the plurality of records that have a corresponding point in time after a completion of an orthodontic treatment for the patient; detect, based on processing of the second subset of the plurality of records, tooth movement indicating orthodontic relapse; determine an amount of the orthodontic relapse by comparing one or more tooth positions of the second subset of the plurality of records to one or more corresponding final tooth positions associated with the orthodontic treatment for the patient; and update the timelapse visualization to display the amount of orthodontic relapse.

A one hundred fifty-fourth implementation may further extend any of the one hundred fifth through one hundred fifty-third implementations. In the one hundred fifty-fourth implementation, the processing device is further to identify compliance data associated with a retainer worn by the patient, wherein the compliance data comprises one or more time periods of retainer non-wear; correlate the amount of the orthodontic relapse with the one or more time periods of retainer non-wear; and display, in the timelapse visualization, one or more visual indicators correlating the orthodontic relapse with the one or more time periods of retainer non-wear.

A one hundred fifty-fifth implementation may further extend any of the one hundred fifth through one hundred fifty-fourth implementations. In the one hundred fifty-fifth implementation, the processing device is further to generate one or more predicted records representing prevention of the orthodontic relapse, wherein the timelapse visualization comprises the one or more predicted records representing prevention of orthodontic relapse.

A one hundred fifty-sixth implementation may further extend any of the one hundred fifth through one hundred fifty-fifth implementations. In the one hundred fifty-sixth implementation, the processing device is further to responsive to determining that the amount of the orthodontic relapse satisfies a condition, generate one or more predicted records representing a correction of the orthodontic relapse; and update the timelapse visualization to include the one or more predicted records.

In a one hundred fifty-seventh implementation, a system comprises a memory and a processing device to execute instructions from the memory. The processing device is configured to receive a plurality of records associated with a dental arch of a patient, wherein each record of the plurality of records corresponds to a point in time of a plurality of points in time and to an imaging modality of a plurality of imaging modalities. The processing device is further configured to provide the plurality of records for further processing. The further processing comprises determining, based on processing of at least one record of the plurality of records, a clinical finding for the patient. The further processing comprises determining a primary imaging modality of the plurality of the imaging modalities based at least in part on the clinical finding. The further processing comprises normalizing at least a subset of the plurality of records. The further processing comprises generating a timelapse visualization of the dental arch of the patient, wherein the timelapse visualization represents the normalized subset of the plurality of records displayed in chronological order of the plurality of points in time. The processing device is further configured to receive the timelapse visualization of the dental arch of the patient. The processing device is further configured to output the timelapse visualization for presentation to a display.

A one hundred fifty-eighth implementation may further extend any of the one hundred fifty-seventh implementation. In the one hundred fifty-eighth implementation, the subset of the plurality of records comprises one or more of the plurality of records corresponding to the primary imaging modality.

A one hundred fifty-ninth implementation may further extend any of the one hundred fifty-seventh through one hundred fifty-eighth implementations. In the one hundred fifty-ninth implementation, the subset of the plurality of records comprises a first record corresponding to a first point in time of the plurality of points in time and to a first imaging modality of the plurality of imaging modalities, wherein the subset of the plurality of records comprises a second record corresponding to a second point in time of the plurality of points in time and to a second imaging modality of the plurality of imaging modalities, and wherein the timelapse visualization represents at least one of the first imaging modality or the second imaging modality.

A one hundred sixtieth implementation may further extend any of the one hundred fifty-seventh through one hundred fifty-ninth implementations. In the one hundred sixtieth implementation, the normalizing is performed across the plurality of records for at least one of a size, a scale, a color balance, a brightness, lighting conditions, contrast level, magnification factor, capture angle, or an orientation.

A one hundred sixty-first implementation may further extend any of the one hundred fifty-seventh through one hundred sixtieth implementations. In the one hundred sixty-first implementation, the timelapse visualization of the dental arch is presented in a dental chart.

A one hundred sixty-second implementation may further extend any of the one hundred fifty-seventh through one hundred sixty-first implementations. In the one hundred sixty-second implementation, the further processing further compress identifying a treatment corresponding to the clinical finding; generating one or more predicted records representing the dental arch of the patient affected by the treatment at a future point in time; updating the timelapse visualization of the dental arch of the patient to include the one or more predicted records; and wherein the instructions are further to receive the updated timelapse visualization; and output the updated timelapse visualization for presentation to the display.

A one hundred sixty-third implementation may further extend any of the one hundred fifty-seventh through one hundred sixty-second implementations. In the one hundred sixty-third implementation, the treatment comprises at least one of orthodontic treatment, retainer realignment treatment, restorative treatment, periodontal treatment, behavioral modification, appliance-based treatment, or interdisciplinary treatment.

A one hundred sixty-fourth implementation may further extend any of the one hundred fifty-seventh through one hundred sixty-third implementations. In the one hundred sixty-fourth implementation, the further processing further compromises identifying one or more photographs of the patient, wherein the photographs comprise at least a mouth of the patient; generating one or more predicted photographs of the patient, wherein the one or more predicted photographs represent an improvement of the at least the mouth of the patient affected by the treatment at a corresponding future point in time; and generating, based on the one or more predicted photographs of the patient, a video representing the improvements of the at least the mouth of the patient over time; and wherein the instructions are further to receive the video representing the improvements of the at least the mouth of the patient over time; and output the video representing the improvements of the at least the mouth of the patient over time for presentation to the display.

A one hundred sixty-fifth implementation may further extend any of the one hundred fifty-seventh through one hundred sixty-fourth implementations. In the one hundred sixty-fifth implementation, the one or more predicted records correspond to the primary imaging modality.

A one hundred sixty-sixth implementation may further extend any of the one hundred fifty-seventh through one hundred sixty-fifth implementations. In the one hundred sixty-sixth implementation, generating the one or more predicted records comprises determining an effect of the treatment on a condition of at least a portion of the dental arch of the patient at the future point in time; and applying the effect to a most recent record of the plurality of records.

A one hundred sixty-seventh implementation may further extend any of the one hundred fifty-seventh through one hundred sixty-sixth implementations. In the one hundred sixty-seventh implementation, the effect is determined using artificial intelligence or a predetermined set of rules.

A one hundred sixty-eighth implementation may further extend any of the one hundred fifty-seventh through one hundred sixty-seventh implementations. In the one hundred sixty-eighth implementation, the further processing further comprises generating one or more predicted records representing the dental arch of the patient affected by the clinical finding at a future point in time; and updating the timelapse visualization of the dental arch of the patient to include the one or more predicted records; and wherein the instructions are further to receive the updated timelapse visualization of the dental arch of the patient; and output the updated timelapse visualization for presentation to the display.

A one hundred sixty-ninth implementation may further extend any of the one hundred fifty-seventh through one hundred sixty-eighth implementations. In the one hundred sixty-ninth implementation, the further processing further comprises identifying one or more photographs of the patient, wherein the photographs comprise at least a mouth of the patient; generating one or more predicted photographs of the patient, wherein the one or more predicted photographs represent a deterioration of the at least the mouth of the patient affected by the clinical finding at a corresponding future point in time; and generating, based on the one or more predicted photographs of the patient, a video representing the deteriorations of the at least the mouth of the patient over time; and wherein the instructions are further to receive the video representing the deteriorations of the at least the mouth of the patient over time; and output the video representing the deteriorations of the at least the mouth of the patient over time for presentation to the display.

A one hundred seventieth implementation may further extend any of the one hundred fifty-seventh through one hundred sixty-ninth implementations. In the one hundred seventieth implementation, generating the one or more predicted records is performed based on the processing one or more of the plurality of records and sensor data corresponding to the patient, wherein the sensor data is generated by a multisensory tool, and wherein the sensor data comprises at least one of pressure, temperature, or acceleration.

A one hundred seventy-first implementation may further extend any of the one hundred fifty-seventh through one hundred seventieth implementations. In the one hundred seventy-first implementation, the one or more predicted records represent the dental arch of the patient affected by a secondary clinical finding at the future point in time, wherein the secondary clinical finding is associated with the clinical finding.

A one hundred seventy-second implementation may further extend any of the one hundred fifty-seventh through one hundred seventy-first implementations. In the one hundred seventy-second implementation, the one or more predicted records correspond to the primary imaging modality.

A one hundred seventy-third implementation may further extend any of the one hundred fifty-seventh through one hundred seventy-second implementations. In the one hundred seventy-third implementation, generating the one or more predicted records comprises determining an effect of the clinical finding on a condition of at least a portion of the dental arch of the patient at the future point in time; and applying the effect to a most recent record of the plurality of records.

A one hundred seventy-fourth implementation may further extend any of the one hundred fifty-seventh through one hundred seventy-third implementations. In the one hundred seventy-fourth implementation, the effect is determined using artificial intelligence or a predetermined set of rules.

A one hundred seventy-fifth implementation may further extend any of the one hundred fifty-seventh through one hundred seventy-fourth implementations. In the one hundred seventy-fifth implementation, the effect is determined based on at least one of patient data, familial data, general population data, or statistical analysis associated with the clinical finding.

A one hundred seventy-sixth implementation may further extend any of the one hundred fifty-seventh through one hundred seventy-fifth implementations. In the one hundred seventy-sixth implementation, the effect comprises a potential outcome and a velocity of progression of the clinical finding.

A one hundred seventy-seventh implementation may further extend any of the one hundred fifty-seventh through one hundred seventy-sixth implementations. In the one hundred seventy-seventh implementation, determining the clinical finding for the patient comprises providing, as input, the at least one of the plurality of records to an artificial intelligence model trained to provide an indication of the clinical finding; and receiving, as output from the artificial intelligence model, the indication of the clinical finding.

A one hundred seventy-eighth implementation may further extend any of the one hundred fifty-seventh through one hundred seventy-seventh implementations. In the one hundred seventy-eighth implementation, the clinical finding comprises an indication of at least one of: caries, malocclusion, periodontal disease, tooth wear, gingival recession, tooth fracture, bruxism, temporomandibular joint disorder, structural anomaly, orthodontic relapse, or restorative disorder.

A one hundred seventy-ninth implementation may further extend any of the one hundred fifty-seventh through one hundred seventy-eighth implementations. In the one hundred seventy-ninth implementation, determining the clinical finding for the patient comprises comparing the at least one record of the plurality of records to a corresponding predetermined criterion.

A one hundred eightieth implementation may further extend any of the one hundred fifty-seventh through one hundred seventy-ninth implementations. In the one hundred eightieth implementation, determining the clinical finding for the patient comprises providing, as input, one or more of the plurality of records to an artificial intelligence model trained to provide an indication of the clinical finding; receiving, as output from the artificial intelligence model, a plurality of indications of the clinical finding, each indication corresponding to a record, wherein each indication comprises a confidence level; and identifying the clinical finding with a highest confidence level.

A one hundred eighty-first implementation may further extend any of the one hundred fifty-seventh through one hundred eightieth implementations. In the one hundred eighty-first implementation, the processing device is further configured to receive each indication and the corresponding confidence level.

A one hundred eighty-second implementation may further extend any of the one hundred fifty-seventh through one hundred eighty-first implementations. In the one hundred eighty-second implementation, the primary imaging modality that corresponds to the clinical finding is determined based on a ranking of the plurality of imaging modalities.

A one hundred eighty-third implementation may further extend any of the one hundred fifty-seventh through one hundred eighty-second implementations. In the one hundred eighty-third implementation, the plurality of imaging modalities comprise at least one of: intraoral scan, extraoral scan, near-infrared image, cone beam computed tomography (CBCT), photograph, video, or radiograph.

A one hundred eighty-fourth implementation may further extend any of the one hundred fifty-seventh through one hundred eighty-third implementations. In the one hundred eighty-fourth implementation, the timelapse visualization is an animated representation of the normalized subset of the plurality of records.

A one hundred eighty-fifth implementation may further extend any of the one hundred fifty-seventh through one hundred eighty-fourth implementations. In the one hundred eighty-fifth implementation, generating the timelapse visualization of the dental arch comprises generating, for one or more pairs of chronologically consecutive records in the subset of the plurality of records, one or more intermediate images; and inserting, for each of the one or more pairs, the one or more intermediate images in between the corresponding pair of chronologically consecutive records.

A one hundred eighty-sixth implementation may further extend any of the one hundred fifty-seventh through one hundred eighty-fifth implementations. In the one hundred eighty-sixth implementation, the one or more intermediate images are generated using interpolation or optical flow techniques.

A one hundred eighty-seventh implementation may further extend any of the one hundred fifty-seventh through one hundred eighty-sixth implementations. In the one hundred eighty-seventh implementation, the timelapse visualization comprises data corresponding to at least one of the plurality of imaging modalities other than the primary imaging modality.

A one hundred eighty-eighth implementation may further extend any of the one hundred fifty-seventh through one hundred eighty-seventh implementations. In the one hundred eighty-eighth implementation, the further processing further comprises identifying an area of interest in the at least one of the plurality of imaging modalities other than the primary imaging modality, wherein the area of interest corresponds to the clinical finding; and providing, in the timelapse visualization, a visual indicator corresponding to the area of interest in the at least one of the plurality of imaging modalities other than the primary imaging modality, wherein the visual indicator highlights the area of interest.

A one hundred eighty-ninth implementation may further extend any of the one hundred fifty-seventh through one hundred eighty-eighth implementations. In the one hundred eighty-ninth implementation, the at least one record that is processed to determine the clinical finding has a first imaging modality, and wherein the primary imaging modality is a second imaging modality that is different from the first imaging modality.

A one hundred ninetieth implementation may further extend any of the one hundred fifty-seventh through one hundred eighty-ninth implementations. In the one hundred ninetieth implementation, the further processing further comprises determining, based on the processing of the at least one record of the plurality of records, a plurality of indications of the clinical finding for the patient, wherein each indication of the clinical finding corresponds to an imaging modality of the plurality of imaging modalities; determining a discrepancy between at least two indications of the clinical finding of the plurality of indications of the clinical finding; ranking, based on a correlation between each of the at least two indications of clinical finding and the corresponding imaging modality, each of the at least two indications of the clinical finding; and resolving the discrepancy based on the ranking.

A one hundred ninety-first implementation may further extend any of the one hundred fifty-seventh through one hundred ninetieth implementations. In the one hundred ninety-first implementation, the further processing further comprises determining a confidence for each of the at least two indications of the clinical finding; determining an overall confidence by combining the confidence for each of the at least two indications of the clinical finding; and receiving the overall confidence corresponding to the clinical finding.

A one hundred ninety-second implementation may further extend any of the one hundred fifty-seventh through one hundred ninety-first implementations. In the one hundred ninety-second implementation, the further processing further comprises determining, based on the processing of the at least one record of the plurality of records, a plurality of clinical findings for the patient; generating one or more predicted records representing the dental arch of the patient affected by the plurality of clinical findings at a future point in time; and generating an updated timelapse visualization of the dental arch by updating the timelapse visualization to include the one or more predicted records; and wherein the instructions are further to receive the updated timelapse visualization; and output the updated timelapse visualization for presentation to the display.

A one hundred ninety-third implementation may further extend any of the one hundred fifty-seventh through one hundred ninety-second implementations. In the one hundred ninety-third implementation, the in response to receiving a user interaction indicating a first clinical finding of the plurality of clinical findings, the further processing further comprises determining a subset of clinical findings comprising the plurality of clinical findings other than first clinical finding; generating a second set of one or more predicted records representing the dental arch of the patient affected by the subset of clinical findings at the future point in time; and generating a second updated timelapse visualization of the dental arch by updating the timelapse visualization to include the second set of the one or more predicted records; and wherein the instructions are further to receive the second updated timelapse visualization of the dental arch; and output the second updated timelapse visualization for presentation to the display.

A one hundred ninety-fourth implementation may further extend any of the one hundred fifty-seventh through one hundred ninety-third implementations. In the one hundred ninety-fourth implementation, in response to receiving a user interaction indicating a first clinical finding of the plurality of clinical findings, the further processing further comprises: generating a third set of one or more predicted records representing the dental arch of the patient affected by the first clinical finding at the future point in time; and generating a third updated timelapse visualization of the dental arch by updating the timelapse visualization to include the third set of the one or more predicted records; and wherein the instructions are further to receive the third updated timelapse visualization of the dental arch; and output the third updated timelapse visualization for presentation to the display.

A one hundred ninety-fifth implementation may further extend any of the one hundred fifty-seventh through one hundred ninety-fourth implementations. In the one hundred ninety-fifth implementation, the further processing further comprises generating a first set of one or more predicted records representing the dental arch of the patient affected by the clinical finding at a future point in time; generating a second set of one or more predicted records representing the dental arch of the patient affected by behavior modifications corresponding to the clinical finding at the future point in time; generating a third set of one or more predicted records representing the dental arch of the patient affected by a first treatment corresponding to the clinical finding at the future point in time; generating a fourth set of one or more predicted records representing the dental arch of the patient affected by a second treatment corresponding to the clinical finding at the future point in time; generating a plurality of updated timelapse visualizations of the dental arch of the patient, wherein each updated timelapse visualization comprises the timelapse visualization updated to include one of the first set of one or more predicted records, the second set of one or more predicted records, the third set of one or more predicted records, or the fourth set of one or more predicted records; and wherein the instructions are further to receive the plurality of updated timelapse visualizations; and output the plurality of updated timelapse visualizations for presentation to the display.

A one hundred ninety-sixth implementation may further extend any of the one hundred fifty-seventh through one hundred ninety-fifth implementations. In the one hundred ninety-sixth implementation, the further processing further comprises identifying a first record of the plurality of records corresponding to a first imaging modality of the patient at a first point in time; identifying a second record of the plurality of records corresponding to a second imaging modality of the dental arch of the patient at the first point in time; identifying, based on at least one of the first record or the second record, a position of one or more teeth of the dental arch and an orientation of the one or more teeth of the dental arch; and generating, based on the first record and the second record, a three-dimensional model of the dental arch of the patient, wherein the three-dimensional model comprises the one or more teeth of the dental arch and a jaw of the dental arch.

A one hundred ninety-seventh implementation may further extend any of the one hundred fifty-seventh through one hundred ninety-sixth implementations. In the one hundred ninety-seventh implementation, the further processing further comprises identifying a third record corresponding to a third imaging modality of the plurality of imaging modalities, wherein the third record corresponds to a second point in time following the first point in time; identifying at least one change in the position of the one or more teeth of the dental arch or the orientation of the one or more teeth of the dental arch, wherein the at least one change corresponds to a movement of the jaw of the dental arch; and generating, based on the at least one change, an updated three-dimensional model of the dental arch, wherein the updated three-dimensional model represents a simulation of the movement of the jaw of the dental arch over time.

A one hundred ninety-eighth implementation may further extend any of the one hundred fifty-seventh through one hundred ninety-seventh implementations. In the one hundred ninety-eighth implementation, the further processing further comprises identifying a fourth record corresponding to a fourth imaging modality of the plurality of imaging modalities, wherein the fourth record corresponds to a second point in time following the first point in time; identifying at least one change in the position of the one or more teeth of the dental arch or the orientation of the one or more teeth of the dental arch, wherein the at least one change corresponds to a movement of soft tissue of the dental arch; and generating, based on the at least one change, an updated three-dimensional model of the dental arch, wherein the updated three-dimensional model represents a simulation of the movement of the soft tissue of the dental arch over time.

A one hundred ninety-ninth implementation may further extend any of the one hundred fifty-seventh through one hundred ninety-eighth implementations. In the one hundred ninety-ninth implementation, the further processing further comprises determining a secondary imaging modality of the plurality of imaging modalities; normalizing at least a second subset of the plurality of records, wherein each record of the second subset of the plurality of records corresponds to a subset of the plurality of points in time; and generating a second timelapse visualization of the dental arch of the patient, wherein the second timelapse visualization represents the normalized second subset of the plurality of records displayed in the chronological order of the subset of the plurality of points in time; and wherein the instructions are further to receive the second timelapse visualization; and output the second timelapse visualization for presentation to the display.

A two hundredth implementation may further extend any of the one hundred fifty-seventh through one hundred ninety-ninth implementations. In the two hundredth implementation, determining the secondary imaging modality comprises receiving a user interaction identifying the secondary imaging modality.

A two hundred first implementation may further extend any of the one hundred fifty-seventh through two hundredth implementations. In the two hundred first implementation, the instructions are further to cause the timelapse visualization and the second timelapse visualization to be presented concurrently, wherein at least one point in time of the plurality of points in time of the timelapse visualization matches at least a second point in time of the subset of the plurality of points in time of the second timelapse visualization.

A two hundred second implementation may further extend any of the one hundred fifty-seventh through two hundred first implementations. In the two hundred second implementation, the further processing further comprises identifying a second imaging modality of the plurality of imaging modalities, wherein the plurality of records does not comprise a record corresponding to the second imaging modality; generating, based on the timelapse visualization, a second timelapse visualization of the dental arch of the patient, wherein the second timelapse visualization represents the timelapse visualization in the second imaging modality; and wherein the instructions are further to receive the second timelapse visualization; and output the second timelapse visualization for presentation to the display.

A two hundred third implementation may further extend any of the one hundred fifty-seventh through two hundred second implementations. In the two hundred third implementation, the further processing further comprises identifying, based on the plurality of records associated with the dental arch of the patient, one or more events indicative of a health status of the patient, wherein each of the one or more events corresponds a particular point in time of the plurality of points in time; and displaying, in the timelapse visualization, a visual indicator of at least one of the one or more events, wherein the visual indicator is displayed at the particular point in time corresponding to the at least one of the one or more events.

A two hundred fourth implementation may further extend any of the one hundred fifty-seventh through two hundred third implementations. In the two hundred fourth implementation, the further processing further comprises simulating, using at least one of a virtual articulator or occlusogram data of the patient, one or more forces acting on one or more teeth of the dental arch of the patient, wherein the one or more forces comprises parafunctional forces; generating one or more predicted records representing the dental arch of the patient affected by the one or more forces, wherein the one or more predicted records comprises at least one of a profile view image representing a facial change of the patient; and updating the timelapse visualization to include the one or more predicted records.

A two hundred fifth implementation may further extend any of the one hundred fifty-seventh through two hundred fourth implementations. In the two hundred fifth implementation, the further processing further comprises identifying a second subset of the plurality of records, wherein the second subset comprises one or more records of the plurality of records that have a corresponding point in time after a completion of an orthodontic treatment for the patient; detecting, based on processing of the second subset of the plurality of records, tooth movement indicating orthodontic relapse; determining an amount of the orthodontic relapse by comparing one or more tooth positions of the second subset of the plurality of records to one or more corresponding final tooth positions associated with the orthodontic treatment for the patient; and updating the timelapse visualization to display the amount of orthodontic relapse.

A two hundred sixth implementation may further extend any of the one hundred fifty-seventh through two hundred fifth implementations. In the two hundred sixth implementation, the further processing further comprises identifying compliance data associated with a retainer worn by the patient, wherein the compliance data comprises one or more time periods of retainer non-wear; correlating the amount of the orthodontic relapse with the one or more time periods of retainer non-wear; and displaying, in the timelapse visualization, one or more visual indicators correlating the orthodontic relapse with the one or more time periods of retainer non-wear.

A two hundred seventh implementation may further extend any of the one hundred fifty-seventh through two hundred sixth implementations. In the two hundred seventh implementation, the further processing further comprises generating one or more predicted records representing prevention of the orthodontic relapse, wherein the timelapse visualization comprises the one or more predicted records representing prevention of orthodontic relapse.

A two hundred eighth implementation may further extend any of the one hundred fifty-seventh through two hundred seventh implementations. In the two hundred eighth implementation, the further processing further comprises responsive to determining that the amount of the orthodontic relapse satisfies a condition, generating one or more predicted records representing a correction of the orthodontic relapse; and updating the timelapse visualization to include the one or more predicted records.

A two hundred ninth implementation comprises a method to perform the instructions of any of the one hundred fifty-seventh through two hundred eighth implementations.

A two hundred tenth implementation comprises a non-transitory computer-readable storage medium comprising instructions that, when executed by a processing device, cause the processing device to perform the instructions of any of the one hundred fifty-seventh through two hundred eighth implementations.

### BRIEF DESCRIPTION OF THE DRAWINGS

Aspects and embodiments of the present disclosure will be understood more fully from the detailed description given below and from the accompanying drawings of various aspects and embodiments of the disclosure, which, however, should not be taken to limit the disclosure to the specific aspects or embodiments, but are for explanation and understanding only.

FIG. 1 illustrates a workflow for generating and/or displaying a multimodal timelapse visualization, in accordance with embodiments of the present disclosure.

FIG. 2 illustrates an architecture comprising a set of systems for generating and/or displaying a multimodal timelapse visualization, in accordance with embodiments of the present disclosure.

FIG. 3 shows a block diagram of an example system for generating and/or displaying a multimodal timelapse visualization, in accordance with some embodiments of the present disclosure.

FIG. 4 illustrates a system workflow for generating a multimodal timelapse, in accordance with some embodiments of the present disclosure.

FIG. 5 illustrates a flow diagram of an example method for generating and displaying a timelapse visualization of a dental arch of a patient, in accordance with some embodiments of the present disclosure.

FIG. 6 illustrates an example of a multimodal presentation of a visualization of a simulated outcome of orthodontic treatment generated by the multimodal timelapse system, in accordance with some embodiments of the present disclosure.

FIG. 7 illustrates a block diagram of an example computing device, in accordance with some embodiments of the present disclosure.

FIG. 8A illustrates a tooth repositioning appliance, according to certain embodiments.

FIG. 8B illustrates a tooth repositioning system, according to certain embodiments.

FIG. 8C illustrates a method of orthodontic treatment using a plurality of appliances, according to certain embodiments.

FIG. 9 illustrates a method for designing an orthodontic appliance, according to certain embodiments.

FIG. 10 illustrates a method for digitally planning an orthodontic treatment, according to certain embodiments.

FIG. 11A illustrates a series of palatal expanders that are configured to progressively expand the suture, according to certain embodiments.

FIG. 11B illustrates a passive holder (e.g., retainer) that may be worn, for example, after the series of

FIG. 11A has completed expanding the patient's palate, according to certain embodiments.

### DETAILED DESCRIPTION

Described herein are embodiments for generating and displaying a multimodal timelapse. Dental practitioners face substantial challenges when attempting to visualize and communicate changes in oral health conditions over time. Current practices typically involve examining one imaging modality at a time, such as intraoral scans, x-rays, photographs, or CBCT images. The comparison can be used to support a diagnosis, or to visualize the progression of a disease, for example. Once a dental practitioner has made a diagnosis, the dental practitioner creates a treatment plan and presents it to the patient, along with the risks, benefits, alternatives, and consequences of no treatment. During the treatment plan presentation, the dental practitioner educates the patient about the different treatment options, which can involve the use of abstract terminology and hand-drawn illustrations to communicate the need for treatment and to demonstrate potential outcomes if no treatment is chosen.

The above described approach creates several pain points that hinder effective diagnosis and patient care. The amount of data a dental practitioner considers when making a diagnosis is ever increasing, which can lead to information overload for a dental practitioner and/or the patient. Information overload can occur when practitioners must process increasing amounts of data from multiple sources that may present conflicting information. The data can come from multiple sources and may be in conflict, which may be difficult for dental practitioners to sift through and prioritize. Furthermore, it can be difficult for the dental practitioner to explain the progression of a disease quickly and efficiently to patients. This can make it challenging to assess whether the patient has an adequate understanding of the current state of their oral health to make an informed decision about their treatment. Using current standards, dental practitioners can be ill-equipped to communicate reasons for treatment when patients are not aware there is an issue (e.g., if the patient is asymptomatic, is not experiencing pain, does not recognize bleeding gums as a problem, etc.). Additionally, assessing whether the patient has an adequate understanding of their current oral health state to make an informed treatment decision can be challenging, particularly where patients are asymptomatic or when patients do not recognize symptoms.

Accordingly, aspects and implementations of the present disclosure address the above-noted and other challenges by providing a system and method for generating and/or displaying a timelapse that leverages one or more imaging modalities to provide a visualization of clinical findings in different states over time (e.g., past, present, and/or future), optionally from a variety of angles and/or views. The imaging modalities can include, for example, three-dimensional extraoral scans, sensor data, video, intraoral scans, x-rays, photographs, cone bean computed tomography (CBCT) scans, near infrared images, etc. The visualization can highlight a clinical finding across multiple imaging modalities. In some embodiments, the visualization can include a predictive simulation that displays the progression and/or improvement of a clinical finding with a series of treatment options. In some embodiments, the predictive simulation can also be displayed as a timeline with a series of milestones. Thus, the system and method described herein aids the dental practitioner in determining the best treatment option for a patient, and enables the patient to participate in the treatment planning process. Through co-discovery, the patient can share their concerns and desires regarding treatment, while the dental practitioner can show the patient the advantages and/or disadvantages of different options, by showing images of the potential outcomes or the change of a condition over time.

As an illustrative example, a patient may have previously declined treatment for a caries that a dental practitioner identified on an x-ray. The timelapse system described herein can provide a visualization of the carious lesion growing over time, using radiographs and/or other imaging. Without the multimodal timelapse visualization described herein, the patient would not be able to easily visualize the progression of the carious lesion over time. Furthermore, the incorporation of sensor data (e.g., pressure, temperature, accelerometer, etc.) can provide further insights, improve predictions, and bridge the communication gap between patients and doctors, including in the time between visits.

In some embodiments, the state of a patient's teeth and/or the condition of their oral health can be tracked using milestones on a timeline. Events such as treatment planned events, completed work, failed work, treatment progress, and/or poor outcomes can be displayed and used to navigate the records. Thus, the timelapse system can enable visualization of individual actions and/or combinations of events and their impact on the overall oral health of the patient. In some embodiments, the timelapse system can further enable a dental practitioner to filter the records based on individual items and/or combinations of items, thus adjusting the visualization to meet specific needs. In some embodiments, the milestone tracking can serve as a source of motivation for patients and/or dental practitioners. The timelapse system can show current state milestones and/or predicted future milestones based on treatment acceptance or refusal. The temporal milestone framework can facilitate treatment discussions and informed consent processes.

In some embodiments, as dental work is completed, the multimodal timelapse system can show the change to the patient's dentition over a certain period of time, which can optionally be presented on a timeline to show points in time. For example, the timelapse visualization can show a current dentition state and then show a predicted final dentition state (e.g., after the treatment). Such a timelapse visualization over a timeline can be a source of motivation for the patient and/or the dental practitioner.

In some embodiments, a patient-facing timelapse visualization can be used as patient motivation, education, and/or as a conversation tool. For example, different conditions can be combined and displayed to demonstrate the progression of a more complex condition (e.g., bruxism can consist of occlusal wear, gingival recession, decrease in tooth height, etc.). The timelapse can be used to show that the current state could have ben avoided by changing a series of variables, and/or to show a future state if the issues are not addressed (e.g., if the patient refuses treatment).

In some embodiments (e.g., in the case of discrepancies between imaging modalities), the multimodal timelapse system described herein can weigh multiple clinical findings, determine a priority, and present a single source of truth in the event that there are discrepancies between imaging modalities with an indication of confidence for the detection across image types (e.g., 97% confidence). In some embodiments, the multimodal timelapse system can provide the user with the ability to turn findings on and/or off as needed, and to only predict the future state the user's selections.

In some embodiments, the timelapse system can normalize the images for lighting, shading, and/or color to create nominal surfaces for normal, healthy tissues to allow abnormal structures to be seen more easily.

In some embodiments, the timelapse system can position the records in a standard format (e.g., corresponding to a standard dental tooth chart), which can track progress over time, even if the record sets consist of different types of records (e.g., of different imaging modalities). The standard format positioning functionality can handle different types of records across different dates, such as near infrared images on one date and bitewing radiographs on another date, within a unified standardized interface. Applicant hereby incorporates by reference the following application as if set forth fully here, as an example method and system for aligning records from different imaging modalities to a standardized format: U.S. Provisional Ser. No. 63/742,611 , filed on Jan. 1, 2025.

In some embodiments, artificial intelligence model(s) can consider the specific patient's data, familial data, general population data, and/or other statistical analysis to forecast potential outcomes and velocity of progression of a clinical finding and/or improvement.

In some embodiments, the multimodal timelapse system described herein can use changes to the state of a patient's oral health in any imaging modality and display a clinical finding on the most comprehensive imaging modality available. The multimodal timelapse system can harmonize the appearance and outlines of the conditions in all images within a given timeframe.

Embodiments described herein provide for an improved method and apparatus for generating and/or displaying a timelapse visualization of the changes in a patient's dentition, dental arches, mouth, teeth, etc. By combining information from different imaging sources, the system may provide a more complete picture of oral health conditions than any single modality could offer alone. The timelapse system described herein can provide dental practitioners with a quick and efficient method to communicate complex dental subjects in an easy-to-understand way, thus saving time and facilitating treatment discussions with patients. When discrepancies exist between different imaging modalities, the system may weigh clinical findings, determine priority, and present a unified assessment while maintaining confidence indicators for detections across different image types. Additionally, the timelapse system described herein can provide personalized risks and/or benefits and progression of disease, rather than the one-size-fits-all approach of conventional systems. In embodiments, normalized visualizations that present complex dental conditions in formats that patients can readily understand are provided, moving beyond abstract explanations to concrete visual demonstrations of a patient's oral health status and potential treatment outcomes. Thus, the timelapse system described herein enhances the overall quality of dental care and patient experience.

Predictive modeling capabilities represent another technical advantage of embodiments of the present disclosure, as the system may incorporate patient-specific data, familial history, general population statistics, and/or other analytical factors to forecast potential outcomes and the velocity of disease progression or improvement. The system may adjust progression rates for various conditions and account for factors that may indirectly affect outcomes, such as dietary habits or behavioral patterns. Treatment planning benefits from the ability to visualize multiple treatment scenarios, allowing practitioners to demonstrate trade-offs between different approaches in terms of quality, time, and cost considerations. The system may filter visualizations based on individual conditions or combinations of conditions, preventing information overload while maintaining the ability to show interconnected relationships between different oral health issues.

For example, treatment planning and presentation can be a difficult and time-consuming process when trying to achieve informed consent of different options. With every treatment plan, there are tradeoffs between quality, value, and time, which a patient may not fully understand. For example, a patient may easily understand the need to fix a broken tooth, but may not as easily understand the benefits of putting the teeth in a better position first (e.g., ortho-restorative treatment). Additionally, the system and method described herein can allow for an objective way to describe the potential pathways and treatment outcomes. The timelapse visualization can aid in performing a cost-benefit analysis. A dental practitioner can present a series of treatment plans, or narrow the options to meet the patient's needs or wishes. For example, utilizing the multimodal timelapse system described herein, the dental practitioner can present the patient with timelapse visualization of the following options: (a) no treatment or behavior modifications, (b) no treatment but implements behavior modifications, (c) treatment A, and/or (d) treatment B. The multimodal timelapse visualizations described herein can help facilitate the discussion between the patient and dental practitioner enhance the overall quality of dental care and patient experience.

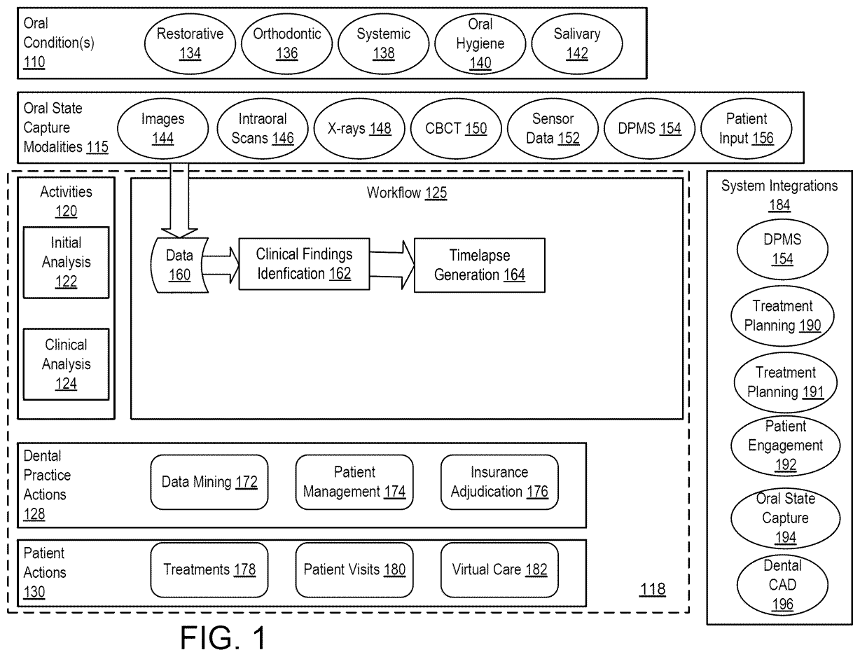

FIG. 1 illustrates a workflow 125 for generating and displaying a multimodal timelapse visualization of the changes in a patient's dentition, dental arches, mouth, teeth, etc. by a multimodal timelapse system 118, in accordance with embodiments of the present disclosure. The workflow 125 may be a general digital workflow covering use of radiographs, intraoral scans, dentition and/or facial photographs (e.g., as taken by a patient of their own dentition or face), and/or other oral state capture modalities within a digital platform of integrated products/services to provide temporal visualizations of clinical findings, assess oral health progression over time, and/or predict future oral health states. The workflow 125 may be used to assist doctors and/or users of a multimodal timelapse system 118 to visualize changes in patient oral health conditions, identify disease progression patterns, determine treatment outcomes, provide predictive simulations of future states with and/or without treatment, facilitate patient education and treatment acceptance, and so on. The workflow 125 may be executed by a digital platform of integrated products in embodiments.

A patient may have one or more oral conditions 110. Oral conditions 110 may include or be related to caries, gum recession, gingival swelling, tooth wear, bleeding, malocclusion, tooth crowding, tooth spacing, plaque, tooth stains, and/or tooth cracks, for example. In some embodiments, the oral conditions 110 may include restorative conditions 134, orthodontic conditions 136, systematic conditions 138, oral hygiene conditions 140, salivary conditions 142, and so on. Restorative conditions 134 may include conditions such as caries that are addressable by performing restorative dental treatment. Such restorative dental treatment may include drilling and filling caries, performing root canals, forming preparations of teeth and applying caps or crowns to the preparations, pulling teeth, adding bridges to teeth, and so on. Restorative conditions may also include results of past restorative treatments of the patient's oral cavity. Examples of past restorations include fillings, caps, crowns, bridges, and so on. Orthodontic conditions may include conditions treatable via orthodontic treatment. Such orthodontic conditions may include a malocclusion (e.g., tooth crowding, overbite, underbite, posterior crossbite, posterior open bite, tooth gaps, etc.). Orthodontic conditions may be associated with restorative conditions in some instances. For example, tooth crowding may cause caries, which results in restorative treatment. Systematic conditions 138 may include conditions such as periodontitis, periodontal bone loss, gum recession, tooth wear, and so on. Systematic conditions 138 may be associated with restorative conditions 134 and/or orthodontic conditions 136.

Oral hygiene conditions 140 may include brushing and flossing related conditions, such as development of calculus on teeth, caries, and so on. Oral hygiene conditions 140 may be related to restorative conditions 134, orthodontic conditions 136 and/or systematic conditions 138 in embodiments. Salivary conditions 142 may include a pH level of a patient's mouth that is outside of normal, a low level of saliva, and so on. Salivary conditions 142 may be related to restorative conditions 134, orthodontic conditions 136, systematic conditions 138 and/or oral hygiene conditions 140 in embodiments. For example, the detection and identification of salivary conditions may be used as an input to an ML model that can use such information to assess periodontal disease, acid reflux, vomiting, poor diet, oral cancer, and/or oropharyngeal cancer. For example, biomarkers of saliva may be used to assist in the assessment and/or management of periodontal disease. Tooth erosion, caries and/or saliva biomarkers may be used to identify acid reflux, vomiting and/or poor diet. In some instances, an oral condition of a patient may include a cross-classification. Such oral conditions may belong to multiple different categories of oral conditions 110. For example, caries may be a restorative condition 134, an orthodontic condition 136 and an oral hygiene condition 140. Oral condition may include oral conditions associate with a dental treatment that has already begun in some embodiments.

A dental practice (e.g., a group practice or solo practice) or a patient may capture data about a patient's oral state using one or more oral state capture modalities 115. A common oral state capture modality used by dental practices are radiographs (i.e., x-rays) 148 generated by radiography machines. There are multiple different types of x-rays that a genal practice may capture of a patient's oral cavity, including bite-wing x-rays, panoramic x-rays and periapical x-rays.

A bite-wing x-ray is a type of dental radiograph used to detect dental caries (cavities) and monitor the health of teeth and supporting bone. During a bite-wing x-ray, the patient bites down on a small tab or wing-shaped device attached to the x-ray film or sensor. This helps keep the film or sensor in place while the x-ray is taken. An x-ray machine (also referred to as a radiography machine) is positioned outside the mouth to capture images of the upper and lower teeth on one side of the mouth at a time. Accordingly, a bite-wing x-ray includes upper and lower teeth of one side of a patient's mouth. In embodiments, bite-wing x-rays are useful for detecting cavities between teeth and for assessing the fit of dental fillings and crowns. Bite-wing x-rays may also be used to help in diagnosing gum disease and/or to monitor bone levels around the teeth in embodiments.

A periapical x-ray, also known as a periapical radiograph, is a type of dental x-ray that focuses on specific areas of the mouth, particularly individual teeth and the surrounding bone. During a periapical x-ray, the dentist or dental radiographer positions an x-ray machine so that it captures detailed images of one or more teeth from crown to root, as well as the surrounding bone structure and supporting tissues. Periapical x-rays may provide a comprehensive view of the entire tooth, including the root tip (apex) and the bone around the tooth's root. In embodiments, periapical x-rays may be used to help diagnose oral health problems such as tooth decay (caries), infections or abscesses at the root of a tooth, bone loss around a tooth due to periodontal (gum) disease, abnormalities in the root structure or surrounding bone, evaluation of dental trauma or injuries, and so on. Periapical x-rays may also be used to assist in assessment of the status of teeth prior to dental procedures such as root canal treatment or extraction.

A panoramic x-ray, also known as a panoramic radiograph or orthopantomogram (OPG), is a type of dental radiograph that provides a comprehensive view of the entire mouth, including the teeth, jaws, temporomandibular joints (TMJ), and surrounding structures in a single image. During a panoramic x-ray, the patient stands or sits in an upright position while an x-ray machine rotates around their head in a semi-circle. The x-ray machine captures a continuous image as it moves, creating a detailed panoramic view of the entire oral and maxillofacial region. In embodiments, a panoramic x-ray may be used to assist in evaluation of the development and position of teeth, including impacted teeth, assessing the health of the jawbone and surrounding structures, detecting cysts, tumors, or other abnormalities in the jaw or adjacent tissues, planning orthodontic treatment by assessing tooth alignment and development, evaluating the placement and condition of dental implants, and/or diagnosing temporomandibular joint (TMJ) disorders or other jaw-related issues.

Another oral state capture modality that is increasingly common in dental practices are intraoral scans 146, and three-dimensional (3D) models of dental arches (or portions thereof) based on such intraoral scans. Intraoral scans are produced by an intraoral scanning system that generally includes an intraoral scanner and a computing device connected to the intraoral scanner by a wired or wireless connection. The intraoral scanner is a handheld device equipped with one or more small cameras and/or optical sensors. The dentist or dental professional moves the intraoral scanner around the patient's mouth, capturing multiple 3D images or scans of the teeth and surrounding structures from various angles. As the intraoral scanner captures the images or scans, they may be processed and displayed on a computer screen in real-time or near real-time. The collected images or scans are stitched together to create a complete 3D digital model of the patient's teeth and oral cavity. This digital impression can be manipulated, analyzed, and shared electronically with dental laboratories or specialists as needed.

An intraoral scan application executing on the computing device of an intraoral scanning system may generate a 3D model (e.g., a virtual 3D model) of the upper and/or lower dental arches of the patient from received intraoral scan data (e.g., images/scans). To generate the 3D model(s) of the dental arches, the intraoral scan application may register and stitch together the intraoral scans generated from an intraoral scan session. In one embodiment, performing image registration includes capturing 3D data of various points of a surface in multiple intraoral scans, and registering the intraoral scans by computing transformations between the intraoral scans. The intraoral scans may then be integrated into a common reference frame by applying appropriate transformations to points of each registered intraoral scan.

In one embodiment, registration is performed for each pair of adjacent or overlapping intraoral scans. Registration algorithms may be carried out to register two adjacent intraoral scans for example, which essentially involves determination of the transformations which align one intraoral scan with the other. Registration may involve identifying multiple points in each intraoral scan (e.g., point clouds) of a pair of intraoral scans, surface fitting to the points of each intraoral scans, and using local searches around points to match points of the two adjacent intraoral scans. For example, the intraoral scan application may match points, edges, curvature features, spin-point features, etc. of one intraoral scan with the closest points, edges, curvature features, spin-point features, etc. interpolated on the surface of the other intraoral scan, and iteratively minimize the distance between matched points. Registration may be repeated for each adjacent and/or overlapping scans to obtain transformations (e.g., rotations around one to three axes and translations within one to three planes) to a common reference frame. Using the determined transformations, the intraoral scan application may integrate the multiple intraoral scans into a first 3D model of the lower dental arch and a second 3D model of the upper dental arch.

The intraoral scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Oral state capture modalities 115 may additionally or alternatively include one or more types of images 144 (e.g., 2D and/or 3D images) of a patient's oral cavity. For example, patient's may generate 2D or 3D images of their dentition using a patient device (e.g., a mobile phone or tablet computer of the patient), and may upload the image(s) via a virtual dental care application, which may execute on the patient device. In some instances, patients may generate images of their oral cavity based on the instruction of an application or service such as a virtual dental care application or service. In some cases, images of a patient's oral cavity (e.g., those taken by a dental practitioner or by a patient themselves) may be taken while the patient wears a cheek retractor to retract the lips and cheeks of the patient and provide better access for dental imaging (i.e., for intraoral photography). Dental practices may additionally include cameras for generating 3D images of a patient's oral cavity and/or cameras for generating 2D images of a patient's oral cavity.

Some dental practices also use cone beam computed tomography (CBCT) 150 as an oral state capture modality 115. CBCT is a medical imaging technique that uses a cone-shaped X-ray beam to create detailed 3D images of the dental and maxillofacial structures. CBCT scanners may be specifically designed for imaging the head and neck region, including the teeth, jawbones, facial bones, and surrounding tissues. A CBCT machine emits a cone-shaped X-ray beam that rotates around the patient's head. A detector on the opposite side of the machine captures a sequence of X-ray images from different angles. The x-ray images are processed to reconstruct them into a detailed 3D volumetric dataset. This dataset provides a comprehensive view of the patient's oral anatomy in three dimensions. CBCT scans may facilitate accurate diagnosis of various dental and maxillofacial conditions, including impacted teeth, dental infections, bone abnormalities, and temporomandibular joint disorders. In embodiments, CBCT imaging may be used for various dental and maxillofacial applications, including implant planning, orthodontic treatment planning, endodontic evaluations, oral surgery, and periodontal assessments.

For image-based oral state capture modalities, multiple depictions and views of the oral cavity and internal structures can be captured (e.g., in radiographs, intraoral scans, etc.). Examples of views include occlusal views, buccal views, lingual views, proximal-distal views, panoramic views, periapical views, bitewings views, and so on.

Oral state capture modalities 115 may additionally or alternatively include sensor data 152 from one or more worn sensors. In some instances, a patient may be prescribed a compliance device (e.g., an electronic compliance indicator), an orthodontic aligner, a palatal expander, a sleep apnea device, a night guard, a retainer, or other dental appliance to be worn by the patient. Any such dental appliance may include one or more integrated sensors, which may include force sensors, pressure sensors, pH sensors, sensors for measuring saliva bacterial content, temperature sensors, contact sensors, bio sensors, and so on. Sensor data from the sensor(s) of a dental appliance worn by a patient may be reported to multimodal timelapse system 118 in embodiments.

In some embodiments, multimodal timelapse system 118 may include one or more system integrations 184 with external systems, which may or may not be dental related. Such system integrations 184 may be for data to be provided to the multimodal timelapse system 118 and/or for the multimodal timelapse system 118 to provide data to the other system(s).

Dental practices generally use a dental practice management system (DPMS) 154 for managing the dental practices. A DPMS 154 is a software solution designed to streamline and automate various administrative and clinical tasks within a dental practice. DPMS 154 are tailored for the needs of dental offices and help dentists and their staff manage patient information, appointments, billing, and other aspects of dental practice management efficiently. A DPMS 154 allows a dental practice to maintain comprehensive patient records, including demographic information, medical history, treatment plans, and clinical notes. The DPMS 154 provides a centralized database that enables dental staff to access patient information quickly and efficiently. DPMS 154 generally includes features for scheduling patient appointments, managing appointment calendars, and sending appointment reminders to patients. DPMS 154 provides tools for creating and managing treatment plans for patients, including digital charting of dental procedures, diagnoses, and treatment progress. This helps dentists and hygienists track patient care effectively and ensure continuity of treatment. DPMS 154 may help to automate billing processes, including generating invoices, processing payments, and managing insurance claims. It can also verify patient insurance coverage, estimate treatment costs, and submit claims electronically to insurance providers for faster reimbursement. DPMS 154 may generate financial reports and analytics to help dental practices track revenue, expenses, and profitability.

In embodiments, data from a DPMS 154 is used as one type of oral state capture modality 115. Multimodal timelapse system 118 may interface with a DPMS 154 to retrieve patient records for a patient, including past oral conditions of the patient, doctor notes, patient information (e.g., name, gender, age, address, etc.), and so on.

In addition to an ability to ingest data from a DPMS 154, multimodal timelapse system 118 in embodiments may be able to generate reports and/or other outputs that can be ingested by the DPMS 154. Accordingly, once the multimodal timelapse system 118 performs an assessment of a patient's oral conditions, oral health problems, treatment recommendations, etc., the multimodal timelapse system 118 may format such data into a format that can be understood by the DPMS 154. The dental treatment assessment system may then automatically add new data entries to the DPMS 154 for a patient based on an analysis of patient data from one or more oral state capture modalities 115.

The multimodal timelapse system 118 may have a system integration with one or more oral state capture systems (e.g., such as an intraoral scanner or intraoral scanning system) 194, from which intraoral scans 146, images 144, 3D models, and/or data from other oral state capture modalities may be received. Examples of oral state capture systems include an intraoral scanning system, a radiograph system or machine, a CBCT machine, and so on.

In embodiments, an output of multimodal timelapse system 118 may be provided to a dental computer aided drafting (CAD) system 196, such as Exocad® by Align Technology. The dental CAD system 196 may be used for designing dental restorations such as crowns, bridges, inlays, onlays, veneers, and dental implant restorations. The dental CAD system 196 may provide a comprehensive suite of tools and features that enable dental professionals to create precise and customized dental restorations digitally. The dental CAD system 196 may import digital impressions (e.g., 3D digital models of a patient's dental arches) captured using intraoral scanners, and may further import data on a patient's oral health from multimodal timelapse system 118. For example, the multimodal timelapse system 118 may export a report on a patient's treatment progress to the dental CAD system 196, which may be used together with a digital impression of the patient's dental arches to develop an appropriate restoration for the patient, for implant planning, for planning of surgery for implant placement, for updating dental treatment, and so on.

In embodiments, multimodal timelapse system 118 may have a system integration 184 with a patient engagement system (e.g., which may include a patient portal and/or patient application) 192. The patient portal may be a portal to an online patient-oriented service. Similarly, the patient application may be an application (e.g., on a patient's mobile device, tablet computer, laptop computer, desktop computer, etc.) that interfaces with a patient-oriented service.

In an example, multimodal timelapse system 118 may integrate with a virtual care system. The virtual care system may provide a suite of digital tools and services designed to enhance patient care and communication between orthodontists/dentists and their patients. The virtual care system may leverage technology to facilitate remote monitoring, consultation, and treatment planning, allowing patients to receive dental care more conveniently and effectively.

In one embodiment, the patient engagement system 192 is or includes a virtual care system that may provide remote monitoring, teleconsultation, treatment planning, patient education and engagement, data management, and data analytics. With respect to remote monitoring, the virtual care system enables orthodontists and dentists to remotely monitor their patients'treatment progress (e.g., for orthodontic treatment) using advanced digital tools. This may include the use of smartphone apps, patient portals, or other software platforms that allow patients to capture and upload photos or videos of their teeth and orthodontic appliances. Such patient uploaded data may be provided to multimodal timelapse system 118 for automated assessment in embodiments. With regards to patient education and engagement, the virtual care system may provide reports, presentations, etc. generated by multimodal timelapse system 118 to patients (e.g., via a patient portal and/or application). For example, the multimodal timelapse system 118 may automatically generate informational videos, treatment progress trackers, compliance reminders, reports, presentations, and so on that are tailored to a patient's oral health and/or to a determination of patient non-compliance, which may be provided to the patient via the patient portal and/or application.

In embodiments, multimodal timelapse system 118 may have a system integration 184 with one or more treatment planning system 190 and/or treatment management system 191 such as ClinCheck® provided by Align Technology®. For example, multimodal timelapse system 118 may have a system integration with an orthodontic treatment planning system and/or with a restorative dental treatment planning system. A treatment planning system 190 may use digital impressions and/or a report output by multimodal timelapse system 118 to plan an orthodontic treatment and/or a restorative treatment (e.g., to plan an ortho-restorative treatment). The treatment planning system 190 may plan and simulate orthodontic and/or restorative treatments. Treatment management system 191 may then receive data during treatment and determine updates to the treatment based on the treatment plan and the updated data.

In an example, an orthodontic treatment planning system may use advanced 3D imaging technology to create virtual models of patients'teeth and jaws based on digital impressions or intraoral scans. These digital models may be used to plan and simulate the entire course of orthodontic treatment, including the movement of individual teeth and the progression of treatment over time. Orthodontists can specify the desired tooth movements, treatment duration, and other parameters, taking into account a report provided by multimodal timelapse system 118, to create personalized treatment plans tailored to each patient's unique anatomy, oral health, and preferences. The orthodontic treatment planning system enables orthodontists to simulate the step-by-step progression of orthodontic treatment virtually, showing patients how their teeth will gradually move and align over the course of treatment. Orthodontists can visualize the planned tooth movements in 3D and make adjustments as needed to optimize treatment outcomes. The orthodontic treatment planning system may provide orthodontists and patients with visualizations of the predicted treatment outcomes, including before-and-after simulations that demonstrate the expected changes in tooth position and alignment, and how those changes might affect the patient's overall oral health as optionally predicted by the multimodal timelapse system 118. These visualizations help patients understand the proposed treatment plan and make informed decisions about their orthodontic care.

During treatment, updated data may be gathered about a patient's dentition, and such data (e.g., in the form of one or more oral state capture modalities 115) may be processed by the multimodal timelapse system 118, optionally in view of an already generated orthodontic treatment plan, to generate an updated report of the patient's overall oral health and/or a treatment progress report (e.g., that may indicate whether the treatment is on-track or off-track as a whole and/or for one or more teeth, that may indicate why treatment is off-track, and so on). The updated report may be provided by the multimodal timelapse system 118 to the orthodontic treatment planning system and/or orthodontic treatment management system to enable the orthodontic treatment planning/management system to perform informed modifications to the treatment plan. Thus, integration of the dental treatment assessment system with the orthodontic treatment planning system and/or treatment management system supports an iterative design process, allowing orthodontists to review and refine treatment plans based on patient feedback, clinical considerations, treatment progress, and automated reports output by multimodal timelapse system 118. Additionally, the updated report may be provided to the patient engagement system 192 (e.g., to a virtual dental care system) for sharing with the patient.

Accordingly, multimodal timelapse system 118 may perform treatment planning and/or management on its own and/or based on integration with one or more treatment planning systems for planning and/or managing orthodontic treatment, restorative treatment, and/or ortho-restorative treatment. An output of such planning may be an orthodontic treatment plan, a restorative treatment plan, and/or an ortho-restorative treatment plan. A doctor may provide one or more modifications to the generated treatment plan, and the treatment plan may be updated based on the doctor modifications.

In addition to those systems mentioned herein that multimodal timelapse system 118 may integrate with, multimodal timelapse system 118 may integrate with any system, application, etc. related to dentistry and/or orthodontics.

Multimodal timelapse system 118 may execute a workflow 125 that includes processing and analysis of data 160 from one or more oral state capture modalities 115. The workflow 125 may include analysis of data 160 at block 162 to identify clinical findings and/or oral health conditions in embodiments. In some embodiments, the workflow 125 may include preprocessing of data 160 to perform segmentation, registration, and/or normalization operations. As a result of such preprocessing, the multimodal timelapse system 118 may establish spatial correspondence between records captured at different time points and across different imaging modalities. Such preprocessing can be performed on individual teeth, anatomical regions, and/or soft tissue landmarks to enable accurate temporal comparisons. Additionally, processing logic may identify anatomical landmarks using automated image analysis techniques to support cross-modal registration even in edentulous cases where traditional tooth-based landmarks are unavailable. Based on the preprocessed data, the multimodal timelapse system 118, at block 12, may analyze the records to detect oral health conditions such as caries, periodontal disease, tooth wear, malocclusion, bruxism, and/or other clinical findings using artificial intelligence models and/or predetermined criteria. Clinical findings may be associated with confidence levels and correlated across multiple imaging modalities to resolve discrepancies and provide unified assessments. One of more of the operations of the workflow 125 may be performed by and/or assisted by application of artificial intelligence and/or machine learning models in embodiments. Multiple embodiments are discussed with reference to machine learning (ML) and artificial intelligence (AI) models herein.

In some embodiments, the workflow 125 may include performing multimodal clinical finding identification and/or primary imaging modality selection. To perform clinical finding identification, the multimodal timelapse system 118 may process multiple imaging modalities using trained machine learning models and rule-based algorithms that analyze geometric features, density patterns, color variations, and/or other modality-specific characteristics to detect and classify oral health conditions. In some embodiments, the multimodal timelapse system 118 may determine a primary imaging modality based on the identified clinical findings, selecting the modality that provides optimal visualization and/or diagnostic accuracy for each specific condition. For example, radiographic imaging may be selected for caries visualization while photographic imaging may be selected for gingival conditions to enhance patient comprehension.

In embodiments, at block 164, the multimodal timelapse system 118 can generate a timelapse visualization including the normalized data 160 and the clinical findings identified at block 162. The timelapse visualization may include temporal sequences showing disease progression or improvement, predictive simulations of future states based on treatment scenarios or natural progression, treatment outcome comparisons, and/or milestone-based timeline displays with significant clinical events. The visualization may include smooth transitions between time points through interpolation techniques and optical flow algorithms, and may incorporate data from multiple imaging modalities while displaying results in a primary modality optimized for clinical accuracy and patient education, in embodiments. The timelapse visualization may demonstrate bone remodeling simulation, soft tissue changes, and/or virtual articulator functionality to predict forces on teeth and potential complications, in embodiments.

In embodiments, a report may be generated including the data 160 and/or outputs of blocks 162 and/or 164. The report may include labeled images, a dental chart, notes, annotations, and/or other information. The report may include a dynamic presentation (e.g., a video) that shows progression of dental conditions over time in some embodiments. The report may be stored in a data store and/or exported to one or more other systems (e.g., DPMS 154, treatment planning system 190, patient engagement system 192, dental CAD system 196).

The dental treatment assessment system 128 may perform multiple dental practice actions 128 and/or patient actions 130 in addition to, or instead of, storing a generated report and/or exporting the report to other systems. Examples of dental practice actions 128 that may be performed include data mining 172, patient management 174 and/or insurance adjudication 176. Examples of patient actions 128 that may be performed include treatments 178, patient visits 180, and/or virtual care 182. One or more of the actions may be performed based on leveraging external systems in embodiments. For example, virtual care 182 may be performed based on leveraging a patient portal and/or application of a virtual dental care system. Patient visits 180 may be performed based on leveraging a DPMS 154. Treatments 178 may be performed based on leveraging a treatment planning system 190 for planning, tracking and/or management of a treatment. Patient management 174 and/or insurance adjudication 176 may be performed based on leverage of a DPMS 154.

Data mining 172 may include analysis of patient data of a dental practice in embodiments. Data mining may be performed for a single dental practice or for multiple different dental practices. Data mining may be performed to refine treatment planning and/or treatment management in embodiments.

Patient management 174 for a dental practice may include a range of tasks and processes aimed at providing quality care and ensuring positive experiences for patients throughout their interactions with the dental practice. Patient management may include appointment scheduling, patient registration and check-in, medical and dental history and records management (e.g., including information about past treatments, allergies, medications, and relevant medical conditions for each patient), treatment planning and coordination, financial management and billing (e.g., including collecting payments, processing insurance claims, providing cost estimates, and discussing payment options or financing arrangements with patients), patient communication and education (e.g., providing information about treatments, procedures, and oral hygiene instructions, as well as addressing patient concerns, answering questions, and maintaining open lines of communication throughout the treatment process), follow-up and recall, and patient satisfaction and feedback management.

The multimodal timelapse system 118 may perform multiple clinical practice actions and patient engagement activities in addition to, or instead of, generating and displaying timelapse visualizations. Examples of clinical practice actions that may be performed include treatment planning support through comparative visualization of multiple treatment scenarios, informed consent facilitation by demonstrating risks and benefits of treatment options, progress monitoring through milestone tracking, outcome prediction using patient-specific data and population statistics, and forensic documentation for legal or insurance purposes. Examples of patient engagement activities that may be performed include motivational visualization generation showing positive treatment outcomes, treatment acceptance enhancement through demonstration of consequences of no treatment, virtual care support through remote monitoring and patient education, email, texting, and/or social media sharing capabilities for positive motivational content. One or more of these actions may be performed based on leveraging external systems in embodiments. For example, treatment planning may be performed based on leveraging treatment data 153 and patient data 156 including familial history and behavioral factors. Patient engagement may be performed based on leveraging social media platforms, email systems, and/or patient portal integration for virtual care delivery and remote patient motivation.

Insurance adjudication 176 for a dental practice refers to the process of evaluating and determining the coverage and reimbursement for dental services provided to patients by their dental insurance carriers. Insurance adjudication 176 involves submitting claims to insurance companies, reviewing the claims for accuracy and completeness, and processing them according to the terms of the patient's insurance policy. After providing dental services (e.g., treatment) to a patient, the dental practice submits a claim to the patient's insurance company electronically or via paper.

Some of the analyses that are performed to assess the patient's dental health and/or treatment progression analyses that compare dentition states of the patient at multiple different points in time. Time-based comparative analyses that may be performed include a time-based comparison of gum recession, a time-based comparison of tooth wear, a time-based comparison of tooth movement, a time-based comparison of tooth staining, and so on. In some embodiments, processing logic automatically selects data collected at different points in time to perform such time-based analyses. Alternatively, a user may manually select data from one or more points in time to use for performing such time-based analyses.

In one embodiment, different types of analyses are performed that include analysis for tooth cracks, gum recession, tooth wear, occlusal contacts, crowding and/or spacing of teeth and/or other malocclusions, plaque, tooth stains, calculus, bone loss, bridges, fillings, implants, crowns, impacted teeth, root-canal fillings, caries, and/or dental treatment progression. Additional, fewer and/or alternative oral conditions may also be analyzed and reported. In embodiments, multiple different types of analyses are performed to determine presence, location and/or severity of one or more of the oral conditions. One type of analysis that may be performed is a point-in-time analysis that identifies the presence and/or severity levels of one or more oral conditions at a particular point-in-time based on data generated at that point-in-time. Another type of analysis that may be performed is a time-based analysis that compares oral conditions at two or more points in time to determine changes in the oral conditions, progression of the oral conditions and/or rates of change of the oral conditions. For example, in embodiments a comparative analysis is performed to determine differences between dentition images taken at different points in time. The differences may be measured to determine an amount of change, and the amount of change together with the times at which the dentition images were taken may be used to determine a rate of change. This technique may be used, for example, to identify an amount of change and/or a rate of change for tooth positions, tooth wear, staining, plaque, crowding, spacing, gum recession, caries development, tooth cracks, and so on.

In embodiments, one or more trained models are used to perform at least some of the mentioned analyses. The trained models may include physics models, statistical models, and/or machine learning models, for example.

In one embodiment, intraoral data from one or more points in time are input into one or more trained machine learning models that have been trained to receive the intraoral data as an input and to output classifications of one or more types of oral conditions, and/or of treatment progress. In one embodiment, the trained machine learning model(s) is trained to identify areas of interest (AOIs) from the input intraoral data and to classify the AOIs based on oral conditions. The AOIs may be or include regions associated with particular oral conditions. The regions may include nearby or adjacent pixels or points that satisfy some criteria, for example. The intraoral data that is input into the one or more trained machine learning model may include three-dimensional (3D) data and/or two-dimensional (2D) data. The intraoral data may include, for example, one or more 3D models of a dental arch, one or more projections of one or more 3D models of a dental arch onto one or more planes (optionally comprising height maps), one or more x-rays of teeth, one or more CBCT scans, a panoramic x-ray, near-infrared and/or infrared imaging data, color image(s), ultraviolet imaging data, intraoral scans, one or more bitewing x-rays, one or more periapical x-rays, and so on. If data from multiple imaging modalities are used (e.g., panoramic x-rays, bitewing x-rays, periapical x-rays, CBCT scans, 3D scan data, color images, and NIRI imaging data), then the data may be registered and/or stitched together so that the data is in a common reference frame and objects in the data are correctly positioned and oriented relative to objects in other data. One or more feature vectors may be input into the trained model, where the feature vectors include multiple channels of information for each point or pixel of an image. The multiple channels of information may include color channel information from a color image, depth channel information from intraoral scan data, a 3D model or a projected 3D model, intensity channel information from an x-ray image, and so on.

In embodiments, image processing and/or 3D data processing may be performed on image data and/or other dental data. Such image processing and/or 3D data processing may be performed using one or more algorithms. The image processing may include performing automated measurements such as size measurements, distance measurements, amount of change measurements, rate of change measurements, ratios, percentages, and so on.

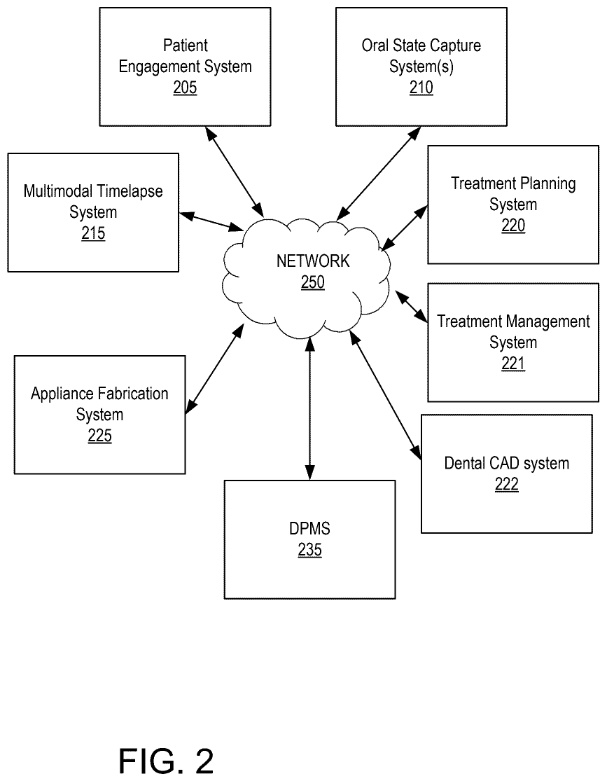

FIG. 2 illustrates an architecture comprising a set of systems for generating and/or displaying a multimodal timelapse visualization, in accordance with embodiments of the present disclosure. The systems in one embodiment include a patient engagement system 205, one or more oral state capture systems 210, a treatment planning and/or management system 220, a DPMS 235, an appliance fabrication system 225, and/or a multimodal timelapse system 215.

In embodiments, multimodal timelapse system 215 corresponds to multimodal timelapse system 118 of FIG. 1. Furthermore, in embodiments patient engagement system 205 corresponds to corresponds to patient engagement system 192. In further embodiments, treatment planning system 220 may correspond to treatment planning system 190 and/or treatment management system 221 may correspond to treatment management system 191 of FIG. 1. The treatment planning and/or management systems 220, 221 may provide treatment plans, treatment recommendations, orthodontic/restorative integration capabilities, and/or other capabilities. These systems may, in some implementations, take in representations of dentition, identify (through human activities and/or automation) orthodontic/restorative treatments to dentition, provide staging/intermediate positioning/final positioning capabilities of orthodontic/restorative treatments, receive and/or process modifications to the treatment plan, provide updated treatments, support appliance design, etc. In some implementations, the treatment planning systems implement an end-to-end digital treatment planning workflow.

Treatment planning system 220 may provide controls for modifying and/or moving oral structures, teeth, etc. In some embodiments, treatment planning system 220 Includes hard limits on some movements, oral structure positions, etc., and may determine when and/or where certain types of interactions are permitted. This may include comparison of an instructed movement/position of one or more oral structures against entries in hard limit databases that store information about movements that are and/or not feasible.

The treatment management system 221 and/or multimodal timelapse system 215 may provide interactive tools to allow users to plan/manage treatments, examine the state of a person's dentition, evaluate data from x various oral state capture modalities, and so on. The treatment management system 221 and/or multimodal timelapse system 215 can provide users with an immersive experience where they can evaluate and/or annotate a person's dentition, plan/implement possible treatments for the person's dentition, review/approve/implement actions/recommendations, etc. In some implementations, the treatment management system 221 and/or multimodal timelapse system 215 implements one or more standalone tools related to interaction with a segmented radiographic representation of the oral cavity. The multimodal timelapse system 215 may communicate to the treatment planning system 220 and/or treatment management system 221 for ortho-restorative capabilities through APIs or other architecture.

In some implementations, the multimodal timelapse system 215 is combined with ortho-restorative capabilities (e.g., e.g., treatment planning and/or treatment management functionalities). In such implementations, a user might be presented with ortho-restorative capabilities on one or more 3D models of a dental arch (e.g., generated from an intraoral scan scan) of the oral cavity. The 3D model(s) may show teeth represented from an intraoral scan, and may show teeth and other oral structures and/or conditions as determined from a segmented radiographic representation of the oral cavity.

In further embodiments, oral state capture system(s) 210 may correspond to one or more oral state capture systems 194 of FIG. 1. In further embodiments, dental CAD system 222 may correspond to dental CAD system 154 of FIG. 1. In further embodiments, DPMS 235 may correspond to DPMS 154 of FIG. 1.

The appliance fabrication system 225 may include one or more systems that allow appliance design and/or fabrication in embodiments. Appliance fabrication system 225 may include systems for manufacturing dental appliances and/or orthodontic appliances for patients, for example. Such dental and/or orthodontic appliances may include orthodontic aligners (e.g., clear polymeric aligners), palatal expanders, sleep apnea devices, retainers, mouth guards, night guards, and so on. In some embodiments, treatment planning and/or management system 220 generates digital models for one or more molds and/or dental appliances. The digital models for dental appliances may be used to directly print dental appliances using additive manufacturing such as 3D printings. Digital models for molds may be used to print molds associated with dental appliances. Polymeric sheets may then be thermoformed over the printed molds and trimmed to form the dental appliances in embodiments. Appliance fabrication system 225 may include 3D printers, thermoforming machines, automation machines for part movement and handling, quality control stations, and so on.

Some or all of the indicated systems may be connected via a network 250, which may include one or more public networks (e.g., the Internet) and/or private networks (e.g., intranets). Systems may exchange information such are reports, 3D model files, standard tessellation language interface (STI) files for use in 3D printing, and so on.

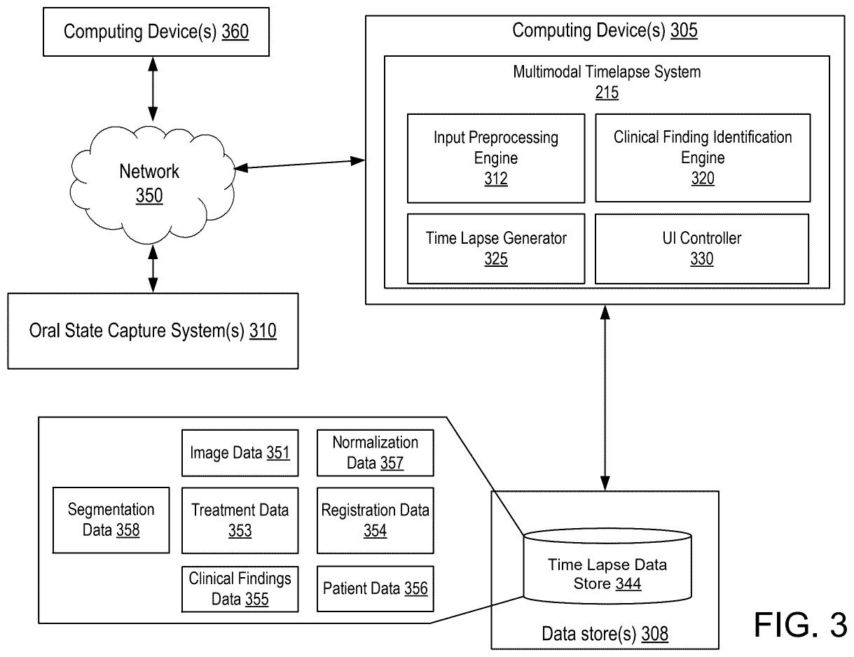

FIG. 3 illustrates a block diagram of an example system 300 for generating and/or displaying a multimodal timelapse visualization, in accordance with some embodiments of the present disclosure. System 300 includes a computing device 305 (e.g., a user device) that may be coupled to one or more computing devices 360 (e.g., server computing devices), oral state capture system(s) 310, and/or a data store 308.

Computing devices 305 and/or 360 may each include a processing device, memory, secondary storage, one or more input devices (e.g., such as a keyboard, mouse, tablet, and so on), one or more output devices (e.g., a display, a printer, etc.), and/or other hardware components. Computing device 305 may be connected to a data store 308 either directly or via a network (e.g., network 350). The network 350 may be a local area network (LAN), a public wide area network (WAN) (e.g., the Internet), a private WAN (e.g., an intranet), or a combination thereof. The computing device 305 may additionally or alternatively be connected to computing device(s) 360 and/or oral state capture systems 310 via a network 350, which may be a local area network (LAN), a public wide area network (WAN) (e.g., the Internet), a private WAN (e.g., an intranet), or a combination thereof. In some embodiments, oral state capture system(s) 310 connect to computing device(s) 305 directly via a wired or wireless connection.

Data store 308 may be an internal data store, or an external data store that is connected to computing device 305 directly or via a network. Examples of network data stores include a storage area network (SAN), a network attached storage (NAS), and a storage service provided by a cloud computing service provider. Data store 308 may include a file system, a database, or other data storage arrangement.

In some embodiments, data store 308 can include a timelapse data store 344. In some embodiments, the timelapse data store 344 can include image data 351 (e.g., intraoral scan data, CBCT data, bite-wing x-rays, panoramic x-rays, periapical x-rays, 3D face scan data, 2D facial images, etc.), treatment data 353, registration data 354, clinical findings data 355, patient data 356, normalization data 357, and/or segmentation data 358. In some embodiments, image data 351, treatment data 353, registration data 354, clinical findings data 355, patient data 356, normalization data 357, and/or segmentation data 358 can reference a patient identifier.

In some embodiments, patient data 356 can include information identifying individual patients, including for example a patient identification number by which other data is referenced. In some embodiments, patient data 356 can include patient-specific information including demographics, medical history, familial history, and/or behavioral factors that may influence oral health outcomes. In some embodiments, patient data 356 can store one or more generated multimodal timelapse visualization(s), e.g., as generated by timelapse generator 325. In some embodiments, patient data 356 can include one or more records for the patient. Each record can reference data corresponding to data of a particular imaging modality (e.g., particular image data) and to a particular point in time. For example, a first record for a patient can include (or reference) intraoral scan data generated a first point in time, a second record for the patient can include (or reference) CBCT data generated at the first point in time, a third record for the patient can include (or reference) x-ray data generated at a second point in time, and so on.

In some embodiments, patient data 356 can include timeline-based milestone tracking functionality. The multimodal timelapse system 215 can track the state of a patient's teeth and oral health conditions using milestones positioned on a temporal timeline. Events such as treatment planning milestones, completed work, failed work, treatment progress, and/or poor outcomes can be displayed and/or used to navigate patient records. In some embodiments, the events can include specific dental procedures, interventions, and/or therapeutic actions that have been identified and/or scheduled for a patient, e.g., based on an identified clinical finding. In some embodiments, patient data 356 can include patient complaint data and/or historical treatment outcome data of the patient. This data can be used to enhance prediction models and/or treatment recommendations.

In some embodiments, image data 351 can include scan data corresponding to one or more scans (e.g., intraoral scan(s), CBCT scan(s), etc.), radiograph data corresponding to one or more x-rays (e.g., panoramic x-rays, bite-wing x-rays, periapical x-rays, etc.), two-dimensional (2D) image data corresponding to one or more photographs (e.g., generated by a camera and/or a smart phone), three-dimensional (3D) facial scan data, and/or other types of image data of a patient's dental arch(es), mouth, and/or face. In some embodiments, the image data 351 may be supplemented by additional data captured by one or more sensors (e.g., generated by a compliance indicator, multisensory tool, etc.). In some embodiments, image data 351 can include data generated by the oral state capture system 310. In some embodiments, image data 351 can be used to generate a virtual model (e.g., a virtual 2D model and/or a virtual 3D model) of a patient's dental arch(es). Each virtual model can reflect the condition of the dental arch at a particular point in time. Each 3D virtual model can include a 3D surface and appearance properties mapped to each point of the 3D surface (e.g., mapped to points on the 3D surface as textures). In some embodiments, image data 351 can be segmented to generate segmentation data 358. In some embodiments, segmentation data 358 can include segmented image data, e.g., as generated by input preprocessing engine 312, a segmented 3D model and/or segmented intraoral scans. Segmentation may be performed using one or more trained AI models (e.g., such as neural networks), which may perform instance segmentation and/or semantic segmentation in embodiments.

Registration data may be generated based on registration of image data to other image data. In embodiments, registration of image data may be performed within an imaging modality and across imaging modalities. Accordingly, x-ray image data may be registered to a 3D model generated from intraoral scan data, for example. In some embodiments, registration data 354 can include registration data generated by input preprocessing engine 312. In some embodiments, registration data 354 can store one or more transformation matrix that indicates the rotations, translations, and/or deformations that will cause one image/3D model (e.g., generated from a first scanning session) to corresponding to another image/3D model (e.g., generated from a second scanning session). In some embodiments, the registration data 354 can store registrations corresponding to individual records of a patient.

In some embodiments, registration data 354 can include transformation parameters, e.g., generated by input preprocessing engine 312 during registration. Transformation parameters can refer to values that define spatial relationships between different coordinate system and/or imaging positions. Transformation parameters can include rotations, translations, scaling factors, and/or deformations needed to align one image or 3D model with another. As an illustrative example, transformation parameters can specify a 15-degree rotation, a 2 millimeter translation along the x-axis, and a 1.2× scaling factor to align an intraoral scan from a first visit with a scan from a second visit.

In some embodiments, clinical findings data 355 can include information indicating clinical findings for a record, e.g., as determined by clinical finding identification engine 320. In some embodiments, normalization data 357 can include data that is used to normalize the records for size, scale, brightness, orientation, and/or color. Normalization data 357 may be generated by performing normalization on data items in image data 351. In embodiments, images within the same imaging modality are normalized (e.g., to one another). In some embodiments, images are normalized across imaging modalities.

In some embodiments, clinical findings data 355 can include anatomical landmark data. An anatomical landmark can refer to an identifiable structural reference point within an oral cavity and/or surrounding tissue. In some embodiments, anatomical landmarks can include individual teeth, tooth surfaces, gingival margins, palatal rugae, tori, muscle attachments, and/or bone contours. In some embodiments, the anatomical landmarks can include jaw joint positions, occlusal contact points, and/or soft tissue boundaries. In some embodiments, anatomical landmarks can be identified by the input preprocessing engine 312 using automated image analysis techniques.

In some embodiments, the anatomical landmark data can originate from image data 351, e.g., captured by oral state capture system(s) 310. For example, the anatomical landmark data can be extracted from intraoral scan data, CBCT scan data, photograph data, x-ray data, and/or other imaging modality data stored in the timelapse data store 344. In some embodiments, the input preprocessing engine 312 can access the imaging data to identify and/or catalog anatomical landmarks. In some embodiments, the input preprocessing engine 312 can generate anatomical landmark data through automated image analysis techniques. For example, the input preprocessing engine 312 can use one or more machine learning models trained to identify anatomical landmark patterns across different imaging modalities. In some embodiments, segmentation algorithms can isolate individual anatomical structures and establish their spatial coordinates within the imaging data. In some embodiments, computer vision techniques can detect consistent anatomical features such as tooth boundaries, gingival margins, and bone contours without requiring machine learning models.

In some embodiments, the anatomical landmark data can enable registration and normalization of records from different imaging sessions by providing consistent reference points. In some embodiments, the anatomical landmark data can facilitate alignment of records to a standardized dental coordinate system regardless of original imaging angles or patient positioning. In some embodiments, the anatomical landmark data can support cross-modal correlation of clinical findings across different imaging modalities. I some embodiments, the anatomical landmark data can maintain spatial consistency in timelapse visualizations even when traditional tooth-based registration is not possible due to missing or severely damaged teeth.

In some embodiments, oral state capture system(s) 310 can include an intraoral scanner, a CBCT scanner (and/or another imaging device, such as a CT scanner), an electronic compliance indicator (ECI) device, a camera, a video camera, a multisensory tool, an x-ray device, and/or optionally a computing device. In some embodiments, the computing device can be part of a scanner in the oral state capture system 310. In some embodiments, the computing device can be part of computing device 360, 305, and/or a separate device (not shown), and the oral state capture system 310 can send captured data (e.g., scan data, image data, video data) for processing on a separate device. In some embodiments, the oral state capture system 310 can include a patient or client device (e.g., a mobile phone or tablet computer) that can take 2D or 3D images and/or videos of the patient's anatomy in a non-clinical setting (e.g., at a patient's home). The oral state capture system 310 can obtain scan data (e.g., stored as scan data 351).

In some embodiments, oral state capture system 310 includes a CBCT machine. A CBCT machine is a type of x-ray machine that uses a cone-shaped x-ray beam to capture data about the patient's anatomy. The CBCT scan can generate multiple (e.g., 150-200) images form a variety of angles. In some embodiments, the data captured can be used to reconstruct a 3D image of the patient's teeth, mouth, jaw, neck, ear, nose and/or throat.

In some embodiments, oral state capture system 310 includes an intraoral scanning system comprising a scanner for obtaining intraoral scans (e.g., 3D data) of a patient's dentition and optionally a computing device. Alternatively, oral state capture system 310 may include an intraoral scanner, and the computing device may connect to the intraoral scanner to effectuate intraoral scanning. In embodiments, the computing device or another computing device of oral state capture system 310 includes an intraoral scan application that processes intraoral scans generated by the intraoral scanner to generate 3D models of the patient's upper and/or lower dental arches.

In some embodiments, the intraoral scanner may include a probe (e.g., a hand held probe) for optically capturing three-dimensional structures. The intraoral scanner may be used to perform an intraoral scan of a patient's oral cavity. An intraoral scan application running on a computing device may communicate with the scanner to effectuate the intraoral scan. A result of the intraoral scan may be scan data 351 that may include one or more sets of intraoral scans, which may include intraoral images. Each intraoral scan may include a two-dimensional (2D) or 3D image that may include depth information (e.g., a height map) of a portion of a dental site. In embodiments, intraoral scans include x, y and z information. In one embodiment, the intraoral scanner generates numerous discrete (i.e., individual) intraoral scans.

In some embodiments, the oral state capture system 310 can include an ECI device. In some embodiments, the ECI device can be used to accurately monitor of a patient's compliance to a prescribed aligner schedule. For instance, an aligner that is ECI-capable can have one or more sensors designed to detect temperature and/or proximity to a patient's tooth. The sensors can pair to a mobile phone, e.g., via a Bluetooth®-enabled “smart” aligner case, and can receive and/or transmit data between the mobile phone and the ECI. In some embodiments, the ECI device can include a pressure sensor that can measure pressure and can convert the measured physical pressure exerted on it into an electrical signal. The pressure sensor on the occlusal surface of the teeth can detect the occlusal force or biting pressure, which can be used to detect bruxism (grinding and/or clenching of the teeth). The pressure sensor can include a sensing element that directly responds to pressure, a transducer that converts the physical change in the sensing element into an electrical signal, a signal conditioning component that can amplify, filter, and/or convert the signal into a digital signal, and/or an output component that can transmit the conditioned signal to a processing device. For example, the pressure sensor can be used to measure and analyze the forces exerted during various dental procedures and treatments, such as occlusal analysis, implantology, orthodontics, prosthodontics, and/or periodontology. In some embodiments, the pressure sensor can measure electrical activity recorded during execution of a sequence of actions (e.g., bruxism-related events such as teeth clenching and teeth grinding, etc., and/or bruxism-unrelated events such as swallowing, lightly nodding the head, lightly shaking the head, speaking, etc.). In some embodiments, the pressure sensor can record a time-averaged value during execution of a particular sequence of actions. The pressure sensor can detect, record, and/or transmit signals to the computing device. The pressure data (e.g., the detected signals) can indicate clenching or grinding of a patient. In some embodiments, the pressure sensor can be attached to a processing device in oral state capture system 310, or can be otherwise connected to a processing device in oral state capture system 310.

In some embodiments, oral state capture system 310 can include sensors such as pressure sensors, temperature sensors, and/or accelerometers. In some embodiments, such sensors may be integrated into a dental appliance, such as a palatal expander, an orthodontic aligner, a night guard, and so on. A pressure sensor can capture bite force measurements and occlusal contact patterns that contribute to understanding of conditions such as bruxism or temporomandibular joint disorder. Temperature sensors can detect inflammatory conditions or changes in oral tissue health. Accelerometers and other motion sensors can track jaw movement patterns and identify parafunctional habits that contribute to tooth wear or other oral health issues. Thus, the data from these (and/or other) sources to provide additional insight and improve prediction accuracy. The sensor data can be stored in scan data 351 and/or integrated with imaging data to create more comprehensive assessments of health conditions and their underlying causes. In some embodiments, the sensor data corresponds to sensor data 152 of FIG. 1.

In some embodiments, oral state capture system 310 is connected to data store(s) 308 either directly or via network 350. In some embodiments, oral state captures system 310 transmits scan data (e.g., CBCT scan data, intraoral scan data, images, sensor data etc.) and/or video recording data to data store 308 for storage therein.

According to an example in which oral state capture system(s) 310 includes an intraoral scanning system, a user (e.g., a practitioner) may subject a patient to intraoral scanning. In doing so, the user may apply an intraoral scanner to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the intraoral scanner may provide scan data 351 to computing device 305 (or to another computing device of oral state capture system 310). The scan data 351 may be provided in the form of intraoral scan data sets, each of which may include 2D intraoral images (e.g., color 2D images) and/or 3D intraoral scans of particular teeth and/or regions of an intraoral site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the intraoral scanner to the computing device 305 (or other computing device) in the form of one or more points (e.g., one or more pixels and/or groups of pixels). For instance, the intraoral scanner may provide an intraoral scan as one or more 3D point clouds. The intraoral scans may each comprise height information.

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions such as malocclusions, temporomandibular joint disorder (TMD), gingival recession, tooth grinding, orthodontic relapse following completion of orthodontic treatment, and so on. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at an intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

In embodiments, intraoral scanning may be performed on a patient's oral cavity during a visitation of a dental office. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. The scan data 351 generated from the intraoral scanning may include 3D scan data, 2D color images, NIR (near infrared) and/or infrared images, and/or ultraviolet images, of all or a portion of the upper jaw and/or lower jaw. The scan data 351 may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Intraoral scanners may work by moving the intraoral scanner inside a patient's mouth to capture all viewpoints of one or more tooth. During scanning, the intraoral scanner is calculating distances to solid surfaces in some embodiments. Each intraoral scan is overlapped algorithmically, or ‘stitched’, with the previous set of scans to generate a growing 3D surface. As such, each scan is associated with a rotation in space, or a projection, to how it fits into the 3D surface.

During intraoral scanning, an intraoral scan application (e.g., executing on computing device 305 or a computing device of oral state capture system 310) may register and stitch together two or more intraoral scans generated thus far from the intraoral scan sessions. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. In embodiments, separate 3D surfaces are generated for the upper jaw and the lower jaw. This process may be performed in real time or near-real time to provide an updated view of the captured 3D surfaces during the intraoral scanning process.

When a scan session or a portion of a scan session associated with a particular scanning role (e.g., upper jaw role, lower jaw role, bite role, etc.) is complete (e.g., all scans for an intraoral site or dental site have been captured), the intraoral scan application may automatically generate a virtual 3D model of one or more scanned dental sites (e.g., of an upper jaw and a lower jaw). The final 3D model(s) may each be a set of 3D points and their connections with each other (i.e., a mesh). To generate a virtual 3D model, the intraoral scan application may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). Registration algorithms are carried out to register two adjacent or overlapping intraoral scans (e.g., two adjacent blended intraoral scans) and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, the intraoral scan application may match points of one scan with the closest points interpolated on the surface of another scan, and iteratively minimize the distance between matched points. Other registration techniques may also be used. Registration data can be stored in data store 308 as a portion of scan data 351, in embodiments.

The intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D model). The intraoral scan application may integrate intraoral scans into a single virtual 3D model (or two virtual 3D models, one for each dental arch) by applying the appropriate determined transformations to each of the intraoral scans. Each transformation may include rotations about one to three axes and translations within one to three planes.

In some embodiments, the intraoral scan application can determine transformation parameters by comparing corresponding anatomical landmarks between different records. In some embodiments, the intraoral scan application can compute transformation matrices that indicate the rotations, translations, and/or deformations to align images from different imaging sessions and/or modalities. In some embodiments, the intraoral scan application can store transformation parameters for each record to maintain the relationship between the original capture position and the standardized position. The transformation parameters can be stored as registration data 354. In some embodiments, each record in patient data 356 can have associated transformation parameters that define its relationship to the standardized coordinate system. In some embodiments, the transformation parameter enable the system to convert between the original capture position and the normalized position used for timelapse visualization. In some embodiments, these parameters can be used to reverse the standardization if needed for specific analysis or display purposes, for example.

In some embodiments, the intraoral scan application can perform soft tissue registration for edentulous cases where teeth are absent or severely damaged. In some embodiments, when processing edentulous records, the intraoral scan application can map soft tissue landmarks to corresponding positions in the standard dental coordinate system. Soft tissue landmarks include, for example, palatal rugae, tori, and/or muscle attachments. The intraoral scan application can extrapolate tooth positions based on the spatial relationship between soft tissue features and typical dental arch anatomy. This landmark-based registration can enable accurate positioning of edentulous records within the timelapse visualization event when traditional tooth-based registration may not be possible. In some embodiments, the soft tissue landmark data can be stored as clinical findings data 355 and used by the timelapse generator 325 to maintain spatial consistency across records captured at different points in time. This approach can be used for full mouth reconstruction cases or forensic applications where historical tooth positions are determined from soft tissue evidence.

The generated virtual 3D model can include color information. In some embodiments, the oral state data 351 can include color information, e.g., from 2D color images captured during the scanning process. The oral state capture system 310 can use the color information to add color texture to the 3D model(s). Once virtual 3D model(s) of the patient's dental arches are generated, they may be stored in data store 308 as a portion of scan data 351 in embodiments.

In some embodiments, computing device 305 is a desktop computer, a laptop computer, a server computer, etc., located at a doctor's office. In some embodiments, computing device 305 is a server computing device (e.g., of a data center) that may be accessed from client devices (e.g., client devices of doctors, patients, etc.). In some embodiments, computing device 305 is a virtual machine. For example, computing device 305 may be a virtual machine that runs in a cloud computing environment.

In some embodiments, computing device 305 includes a multimodal timelapse system 215 that can include several interconnected components that work together to process, analyze, and visualize oral health data across different time points and imaging modalities. Multimodal timelapse system 215 can include an input preprocessing engine 312, a clinical finding identification engine 320, a timelapse generator 325, and/or a UI controller 330. Multimodal timelapse system 215 can include software, hardware, and/or firmware configured to perform one or more operations with respect to generating and/or displaying a multimodal timelapse visualization of a patient's oral health over time.

In some embodiments, input preprocessing engine 312 can be a software program, module, or logic hosted by a device (e.g., computing device 305) to process image data 351. Input preprocessing engine 312 can perform one or more operations on image data 351 to prepare the image data 351 for the clinical finding identification engine 320, the multimodal timelapse generator 325, and/or the UI controller 330. Input preprocessing engine 312 can perform operations such as filtering, stabilizing, cropping, image enhancement (e.g., to sharpen an image), segmentation, and/or other operations. In some embodiments, if image data 351 does not include a 3D model of a dental arch (e.g., includes 2D images or intraoral scans but no 3D models of dental arches), input preprocessing engine 312 can process the 2D image(s) and/or intraoral scan(s) to generate one or more 3D models. In some embodiments, 3D models can be generated from 2D images. In some embodiments, input preprocessing engine 312 can project 3D scan data 351 into 2D, e.g., using a mesh projection algorithm. Input preprocessing engine 312 can segment the 2D scan data using 2D segmentation techniques and/or 3D segmentation techniques. The resulting segmentation can then be back-projected onto the 3D model or intraoral scan(s) if segmentation was performed in 2D, and/or stored in segmentation data 358.

In some embodiments, input preprocessing engine 312 can segment image data 351 into sections of the patient's dental arch. A section can include one or more teeth of the patient and/or a section of the patient's jaw, in embodiments. In some embodiments, input preprocessing engine 312 can receive or otherwise identify a dataset of image data 351 that corresponds to image data of a patient's dental arch in a particular imaging modality. Input preprocessing engine 312 can segment the dataset into multiple sections. In some embodiments, each section corresponds to an individual tooth, or an individual section of the dental arch corresponding to an individual tooth (e.g., if a tooth is missing, the input preprocessing engine 312 can generate segmentation data corresponding to the area of the dental arch corresponding to the missing tooth). Segmentation data 358 can be stored in data store(s) 308.

In some embodiments, input preprocessing engine 312 can include or implement a trained machine learning model that has been trained to perform semantic segmentation and/or instance segmentation of oral structures (e.g., to determine sizes, shapes, locations, tooth numbers, etc. of individual teeth, gingiva, etc.) for one or more image modalities (e.g., 3D models, 2D images, x-rays, etc.). Applicant hereby incorporates by reference the following application as if set forth fully here, as an example of a machine learning dental segmentation system and method, and training of such a machine learning segmentation system: U.S. Pat. Pub. No. 20210196434A1, published on Jul. 1, 2021, issued on Feb. 20, 2024, U.S. Pat. No. 11,903,793 B2.

In some embodiments, input preprocessing engine 312 can store segmentation information for image data 351 as segmentation data 358. In some embodiments, input preprocessing engine 312 can be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, individual teeth, regions of the dental arch corresponding to individual teeth, gingiva, etc. The trained machine learning model of a segmentation module may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects and/or for each instance of a feature and/or object. In one embodiment, a single mask or map includes segmentation information for all identified classes of features and/or objects. Some types of features are location-specific features and are represented in one or more masks. In some embodiments, input preprocessing engine 312 can perform one or more processing and/or computer vision techniques or operations to extract segmentation information from images (e.g., scan data 351). Such image processing and/or computer vision techniques may or may not include the use of trained machine learning models. Accordingly, in some embodiments, input preprocessing engine 312 does not include a machine learning module.

In some embodiments, input preprocessing engine 312 can process scan data 351 to identify anatomical landmarks using automated image analysis techniques. In some embodiments, input preprocessing engine 312 can detect consistent anatomical features across different imaging modalities. In some embodiments, input preprocessing engine 312 can use a machine learning model trained to identify anatomical landmark patterns in the image data 351 for one or more types of imaging modalities (e.g., intraoral scan data, photographs, x-rays, CBCT scans, and/or other image data). In some embodiments, the input preprocessing engine 312 can use segmentation algorithms to isolate individual anatomical structures and establish their spatial coordinates. The anatomical landmarks can be stored as clinical findings data 355 in some embodiments.

In some embodiments, the input preprocessing engine 312 can register and normalize the image data 351. In some embodiments, the input preprocessing engine 312 can register and normalize the image data 351 before segmentation. For instance, the input preprocessing engine 312 can alter the representation of one image so that it matches another image. The input preprocessing engine 312 can normalize the image data 351 for size, scale, brightness, color, orientation, and so on. Normalization data 357 can be stored in data store 308.

In some embodiments, the input preprocessing engine 112 can perform normalization across different imaging modalities by establishing standardized parameters for each modality type. In some embodiments, the input preprocessing engine 112 can identify the specific imaging modality of each record and apply modality-appropriate normalization algorithms. For example, radiographic images can be normalized for intensity values and contrast levels to account for different sensor types and exposure settings. As another example, photographic records can be normalized for color temperature, white balance, and/or lighting conditions to correct for variations in capture environments.

In some embodiments, the normalization process can standardize spatial dimensions across different imaging modalities to enable accurate cross-modal comparison. The input preprocessing engine 112 can apply scaling transformations to align the field of view and magnification levels between modalities such as intraoral photographs and corresponding scan regions. CBCT data can be normalized for voxel spacing and slice thickness to maintain consistent three-dimensional measurements. Intraoral scan data can be normalized for mesh density and surface resolution to enable accurate geometric comparisons.

In some embodiments, the input preprocessing engine 112 can normalize anatomical orientation and positioning across different imaging modalities using anatomical landmarks as reference points. The input preprocessing engine 112 can identify consistent anatomical features visible across multiple modalities and use these landmarks to establish common coordinate systems. For example, tooth boundaries, gingival margins, and/or bone contours can serve as reference points for aligning records from different imaging sources. The normalization process can apply rotational and translational corrections to ensure that corresponding anatomical structures are positioned consistently across modalities.

In some embodiments, the cross-modal normalization can address temporal variations in imaging parameters by establishing baseline standards for each modality type. The input preprocessing engine 112 can maintain reference templates for normal tissue appearance in each imaging modality and normalize patient records against these standards. For example, near-infrared imaging data can be calibrated to standard wavelength responses and intensity scales. The normalization process can account for equipment-specific variations by applying calibration factors derived from known reference standards.

In some embodiments, input preprocessing engine 312 can register image data 351 to other image data 351 using 3D registration and/or 2D image registration techniques. In some embodiments, 3D registration can include capturing 3D data of various points in multiple 3D representations (e.g., in multiple intraoral scans of the same patient), and registering the 3D representations by computing transformations between the 3D representations. In some embodiments, each tooth is separately registered to instances of that same tooth across different image data. By registering each individual tooth, processing logic can offset any tooth movement that may have occurred between imaging of the patient's dentition. In some embodiments, image registration involves identifying multiple points, point clouds, edges, corners, surface vectors, etc., in each image (e.g., 2D image and/or 3D image or point cloud such as an intraoral scan) of an image pair, surface fitting to the points of each image, and using local searches around points to match points of the two images. The registration data 354 can be stored in data store 308.

In some embodiments, the input preprocessing engine 312 can implement standardized formatting by automatically aligning records from different imaging modalities to a standardized dental coordinate system. The system can use predetermined anatomical landmarks to establish a common reference frame. Each of the image data items may have been pre-processed to generate landmark data, normalization data, segmentation data, etc. This data may be used to facilitate registration of image data across image modalities in embodiments. In some embodiments, the input preprocessing engine 312 can transform each record to match a standardized format. This may be performed regardless of the original imaging angle or patient positioning during image capture. The standardized format can correspond to established dental charting conventions, such as the Universal Numbering System or FDI World Dental Federation notation. The input preprocessing engine 312 can automatically identify tooth positions and orient each record to match a standardized view. In some embodiments, the standardized view includes one or more of a front view, a side view, or an occlusal view. The alignment can be performed using image registration techniques that map anatomical features to their corresponding coordinates in the standardized format.

In some embodiments, the input preprocessing engine 312 can maintain spatial relationship between teeth and surrounding structures across different imaging modalities. The input preprocessing engine 312 can account for variations in patent positioning, imaging device orientation, and/or field of view. The input preprocessing engine 312 can scale and/or rotate the records to achieve consistent positioning within the standardized format framework to normalize the image data within imaging modalities as well as across imaging modalities.

In some embodiments, the standardized format positioning can enable direct comparison of records captured at different points in time and/or using different imaging modalities. For example, an intraoral scan from one visit can be positioned alongside a CBCT scan from another visit within the same coordinate system. This enables dentition, individual teeth, oral health issues, etc. to be compared between image data of different imaging modalities. The standardization can facilitate accurate tracking of changes over time regardless of the imaging modality used at any given point in time.

In some embodiments, the clinical finding identification engine 320 can process the image data 351 and/or patient data 356 to identify one or more clinical findings for the patient. A clinical finding can be, for example, caries, malocclusion, periodontal disease, tooth wear, bruxism, temporomandibular joint disorder, structural anomaly, tooth crack, tooth wear, gingival recession, and/or restorative disorder. Some clinical findings can be correlated to other clinical findings. For example, gingival recession can lead to root caries, or malocclusion can lead to temporomandibular joint disorders. Clinical findings can also be the source of multiple issues. For example, bruxism can cause occlusal wear, gingival recession, decrease in tooth height, etc.

In some embodiments, the clinical finding identification engine 320 can include an AI model that is trained to determine an indication of a clinical finding (e.g., of an existing and/or of a predicted clinical finding). In some embodiments, the clinical finding identification engine 320 can compare the patient's record(s) to a predetermined criterion to identify a clinical finding.

In some embodiments, the clinical finding identification engine 320 can provide, as input to an AI model, the image data 351 that corresponds to the patient (or a subset thereof) from a given point in time. The input image data 351 may include raw or preprocessed image data, and may include landmark data, normalization data, segmentation data, and so on in some embodiments. The AI model can be trained to provide an indication of a clinical finding corresponding to particular imaging modality in some embodiments. In some embodiments, the AI model can receive different types of imaging modalities as inputs, and can provide an output with improved accuracy based on processing of the multiple imaging modalities. In some embodiments, multiple AI models are used, where each AI model processes image data of a different imaging modality. Each AI model may generate an output, and those outputs may be combined to determine a final assessment of a patient's oral health. For example, the outputs of multiple AI models may be averaged (e.g., with a weighted or unweighted average) or otherwise combined.

The clinical finding identification engine 320 can receive, as output from the AI model(s), the indication of the clinical finding(s). In some embodiments, the clinical finding(s) 355 can be stored in data store 308. In some embodiments, the AI model can provide multiple indications of a clinical finding. For example, each indication can correspond to a particular imaging modality. For example, the AI model can provide a first indication of a cavity in a first imaging modality (e.g., intraoral scan image), and a second indication of the cavity in a second imaging modality (e.g., NIR image). In some embodiments, to determine whether to include the clinical finding in the timelapse visualization, the clinical finding identification engine 320 can identify the imaging modality that best corresponds to the clinical finding, and can identify the indication that corresponds to that imaging modality. In some embodiments, the clinical finding identification engine 320 can identify the imaging modality that best corresponds to the clinical finding by identifying the imaging modality that provides the highest diagnostic accuracy of the clinical condition identified. In some embodiments, the clinical finding identification engine 120 can determine correspondence based on the inherent strengths and/or limitations of each imaging modality for detecting particular oral health conditions. For example, radiographic imaging can best correspond to subsurface conditions such as caries and bone loss, and photographic imaging can best correspond to surface conditions such as gingival inflammation and tooth discoloration. In some embodiments, the correspondence can be quantified through diagnostic accuracy metrics including sensitivity, specificity, and/or confidence levels associated with each imaging modality for a specific clinical finding. In some embodiments, the clinical finding identification engine 320 can maintain statistical data correlating imaging modalities with detection accuracy for different condition types. The clinical finding identification engine 320 can assign numerical scores representing the diagnostic reliability of each modality for particular clinical findings, in embodiments. For example, CBCT imaging can receive high correspondence scores for bone-related findings while near-infrared imaging can receive high scores for early decalcification detection.

In some embodiments, identifying the imaging modality that best corresponds to the clinical finding can be based on ranking systems that prioritize imaging modalities according to their diagnostic performance for specific clinical conditions. In some embodiments, the clinical finding identification engine 320 can use predetermined ranking hierarchies, e.g., established through clinical validation studies and/or statistical analysis of diagnostic outcomes. The clinical finding identification engine 120 can select the imaging modality with the highest ranking score for the identified clinical finding type. The ranking can consider factors such as detection sensitivity, image resolution, and/or clinical relevance for each modality-condition pairing, in embodiments.

In some embodiments, the clinical finding identification engine 320 can determine the best correspondence through machine learning algorithms trained on large datasets of imaging modality performance across different clinical scenarios. The algorithms can analyze historical diagnostic accuracy data to establish correlation patterns between imaging modalities and clinical finding types. The clinical finding identification engine 120 can apply these learned patterns to select the most appropriate imaging modality for each newly identified clinical finding based on the highest predicted diagnostic confidence.

In some embodiments, after identifying the indication that corresponds to the imaging modality that best corresponds to the clinical finding, the clinical finding identification engine 320 can determine whether that indication satisfies a criterion in order to be included in the timelapse visualization. In some embodiments, the AI model can provide a confidence score for each indication of a clinical finding, the clinical finding identification engine 320 can determine that the indication satisfies the criterion in response to determining that the corresponding confidence score exceeds a threshold value (e.g., is greater than 70%, 80%, or 90% depending on the specific clinical finding and/or imaging modality used). In some embodiments, the clinical finding identification engine 320 can aggregate the multiple indications of the clinical finding to generate an overall indication, and use the aggregate to determine whether the include the clinical finding in the timelapse visualization. In such embodiments, the criterion can be an average of the confidence score threshold values, for example.

In some embodiments, the clinical finding identification engine 320 can identify one or more treatment options corresponding to the identified clinical finding(s). The treatment options can be stored in treatment data 353. In some embodiments, the clinical finding identification engine 320 can categorize each clinical finding by type, severity, and/or location within the dental arch. In some embodiments, clinical findings can be matches against a treatment database containing established treatment protocols for various oral health conditions.

In some embodiments, the clinical finding identification engine 320 can use rule-based algorithms to compare clinical finding parameters with treatment criteria. For example, caries findings can be matched with restoration options based on cavity size and location. As another example, periodontal disease findings can be associated with scaling, root planning, or surgical interventions based on pocket depth measurements.

In some embodiments, the clinical finding identification engine 320 can use one or more machine learning models trained on historical treatment data to identify treatment options. The ML model(s) can consider patient-specific factors such as demographics, gender, age, medical history, and/or previous treatment outcomes. In some embodiments, multiple treatment options can be ranked by predicted success rate and combability with the patient's clinical profile (e.g., as stored in patient data 356).

In some embodiments, the clinical finding identification engine 320 can identify alternative treatment approaches (e.g., alternative to standard clinical or mechanical treatments) and/or behavioral modifications. For example, bruxism findings can be associated with night guard therapy, stress management, and/or orthodontic correction options. In some embodiments, the clinical finding identification engine 320 can consider interdisciplinary treatment approaches that address multiple clinical findings simultaneously.

In some embodiments, the clinical finding identification engine 320 can store identified treatment option(s) in treatment data 353 with associated metadata including expected outcomes, treatment duration, and/or cost considerations. In some embodiments, the timelapse generator 325 can use treatment data to generate predictive visualizations showing potential outcomes for each treatment option.

In some embodiments, the clinical finding identification engine 320 can identify existing and/or predicted orthodontic relapse following completion of orthodontic treatment. In some embodiments, the clinical finding identification engine 320 can process image data 151 to detect tooth movement that occurs after orthodontic treatment completion. In some embodiments, the clinical finding identification engine 320 can measure changes in tooth positions relative to the final treatment positions achieved by an orthodontic treatment plan.

In some embodiments, the clinical finding identification engine 320 can correlate orthodontic relapse measurements with retainer compliance data from sensor data (e.g., from sensor data 152). In some embodiments, the input preprocessing engine 312 can process compliance information from electronic compliance indicators integrated into retainers to determine wear patterns and/or compliance rates. The clinical finding identification engine 320 can determine correlations between periods of poor retainer compliance and subsequent tooth movement and/or relapse patterns, in embodiments.

In some embodiments, the timelapse generator 325 can create a timelapse visualization showing the progression of orthodontic relapse over time to demonstrate the consequences of inadequate retainer wear. The timelapse generator 325 can generate predictive simulations showing how continued poor compliance can lead to further relapse, possibly requiring further orthodontic intervention, in embodiments. In some embodiments, the visualization can highlight specific teeth or regions experiencing the greatest amount of relapse movement.

In some embodiments, the multimodal timelapse system 215 can generate motivational content demonstrating how retainers can realign minor relapse cases (e.g., cases in which the tooth movement measurement after completion of orthodontic treatment is less than a threshold amount), and prevent the need for comprehensive retreatment. In some embodiments, the generated timelapse visualization can show patients the potential benefits of improved retainer compliance by displaying scenarios where consistent retainer wear maintains treatment results. In some embodiments, the UI controller 330 can present the motivational visualization to encourage patient compliance and/or help patients avoid additional orthodontic treatment costs and time commitments.

In some embodiments, the generated predictive visualizations can be displayed in the primary imaging modality that is associated with the clinical finding. That is, in some embodiments, the timelapse generator 325 can generate predictive visualizations that correspond to the same imaging modality selected for the historical timelapse visualization. The timelapse generator 325 can maintain visual consistency by presenting future predictions in the modality that provides optimal visualization for the specific clinical condition, in embodiments.

In some embodiments, the predictive visualizations can be shown in a different imaging modality than the one used for clinical finding detection. For example, the timelapse generator 325 can detect clinical findings using one imaging modality that supports diagnostic accuracy, and generate predictive visualizations in another modality that may offer better patient comprehension. As an illustrative example, the timelapse generator 125 can process x-ray data to detect caries and generate predictive photographs showing how the condition might appear visually to the patient over time.

In some embodiments, the predictive visualizations can incorporate data from multiple imaging modalities while being displayed in a single primary modality. For example, the timelapse generator 325 can use CBCT data to inform bone structure predictions while presenting the results as photographic sequences that demonstrate facial profile changes. As another example, the timelapse generator 125 can combine near-infrared imaging data with intraoral scan information to generate predictive photographs showing surface changes that correlate with subsurface conditions.

In some embodiments, the modality selection for predictive visualizations can be based on the intended audience and/or communication objectives. The timelapse generator 325 can generate predictive visualizations in photographic modalities for patient education purposes while simultaneously creating predictive radiographic sequences for clinical documentation. The UI controller 330 can present different modality versions of the same predictive content to different users based on their roles and/or information needs.

In some embodiments, the timelapse generator 325 can use scan data 351, registration data 356, segmentation data 358, clinical findings data 355, treatment data 353, and/or normalization data 357 to generate a multimodal timelapse visualization of a patient's oral condition over time.

In some embodiments, the timelapse generator 325 can process clinical findings identified by the clinical finding identification engine 320 to create a temporal timelapse visualization of a patient's oral condition. In some embodiments, the timelapse generator 325 can generate a timelapse visualization for various clinical findings, including caries, malocclusion, periodontal disease, tooth wear, bruxism, temporomandibular joint disorder, structural anomalies, and/or restorative disorders. Each clinical finding can be associated with a specific imaging modality (or modalities) that provide optimal visualization for that condition.

In some embodiments, the timelapse generator 325 can use the primary imaging modality associated with a particular clinical finding when generating the timelapse visualization. In some embodiments, the timelapse generator 325 can incorporate data from multiple imaging modalities when generating a timelapse visualization that displays a single primary imaging modality. The timelapse generator 325 can use secondary imaging modalities to enhance the accuracy of the visualization and/or to provide additional clinical context. For example, CBCT data can inform bone structure changes while intraoral scan data can provide surface detail for the same clinical finding.

The timelapse generator 325 can identify and/or retrieve records from patient data 356 that correspond to the identified clinical finding(s) and the determined selected primary imaging modality. The timelapse generator 325 can retrieve records spanning multiple time points to create a chronological sequence showing the progression or improvement of the clinical condition over time.

In some embodiments, the timelapse generator 325 can apply registration data 354 to align records captured at different imaging sessions. The timelapse generator 325 can use transformation matrices that specify rotations, translations, and/or scaling factors to register each record to a common coordinate system. The registration process can ensure spatial consistency across records captured with different imaging angles, distances, and/or patient positioning. In embodiments, the registration process may be performed to register image data from different imaging modalities together. For example, some or all of x-ray data, a 3D model generated from intraoral scan data, CBCT data, 2D color images, and so on may be registered together in a common reference frame.

In some embodiments, the timelapse generator 325 can apply normalization data 357 to standardize visual appearance across records. For example, the timelapse generator 325 can adjust brightness, contrast, color balance, and/or lighting conditions to create a consistent visual presentation. For photographic records, the timelapse generator 325 can correct for different lighting environments such as fluorescent lighting versus natural lighting. For radiographic records, the timelapse generator 325 can normalize intensity values to account for different sensor types and/or exposure settings.

In some embodiments, the timelapse generator 325 can generate intermediate frames between chronologically consecutive records to create smooth temporal transitions. The timelapse generator 325 can use interpolation algorithms and/or optical flow techniques to calculate pixel movements between time points. These intermediate frames can fill temporal gaps and create movie-like visualization rather than abrupt transitions between discrete time points. Applicant hereby incorporates by reference the following application as if set forth fully here, as an example of generating dental treatment videos: U.S. Patent Pub. No. 20240144480A1, published on May 2, 2024. Applicant hereby incorporates by reference the following application as if set forth fully here, as an example of a system and method for generating augmented videos with dental modifications: U.S. Patent Pub. No. 20240185518A1, published on Jun. 6, 2024. Applicant hereby incorporates by reference the following application as if set forth fully here, as an example of integrating video data into image-based dental treatment planning and client device presentation: U.S. patent application Ser. No. 19/179,909, filed on Apr. 15, 2025.

In some embodiments, the timelapse generator 325 can incorporate clinical findings data 355 to highlight specific anatomical regions or conditions within the visualization. The timelapse generator 325 can overlay visual indicators, color maps, and/or annotations to draw attention to areas of clinical interest. For example, caries progression may be highlighted with color coding, and gingival recession may be marked with measurement indicators.

In some embodiments, the timelapse generator 325 can generate predictive frames representing future states of the patient's oral condition. The timelapse generator 325 can use treatment data 353 to model potential outcomes based on different treatment scenarios. In some embodiments, predictive algorithms can calculate disease progression rates, treatment response patterns, and/or healing timelines. These future frames can extend the timelapse beyond historical data to show potential outcomes.

In some embodiments, the timelapse generator 325 can generate multiple timelapse variations corresponding to different treatment options. Each variation can show the predicted progression of the clinical finding under a specific treatment scenario. The timelapse generator 325 can crate comparative visualizations showing side-by-side outcomes for treatment versus no treatment options.

In some embodiments, the timelapse generator 325 can incorporate filtering capability that focus the visualization on specific findings or anatomical regions. The timelapse generator 325 can generate targeted timelapse sequences while maintaining context from surrounding structures. This filtering can help reduce visual complexity and focus patient attention on relevant clinical information.

In some embodiments, the generated timelapse visualization can include temporal controls allowing a user to navigate through different time points, adjust playback speed, and/or pause at specific milestones. The timelapse generator 325 can embed metadata within the visualization indicating capture dates, imaging modalities, and/or clinical findings associated with each frame.

In some embodiments, the timelapse generator 325 can modify the timelapse visualizations, e.g., based on a user interaction received from the user device (e.g., via the UI). In some embodiments, the timelapse generator 325 can receive user interaction data from computing device 305 via UI controller 330. The user interaction data can include selection of one or more specific clinical findings to include or exclude from the timelapse visualization. For example, a user can select individual conditions such as caries, periodontal disease, and/or tooth wear to focus the visualization on particular aspects of oral health.

In some embodiments, the timelapse generator 325 can process the user selection(s) to identify clinical finding(s) to be emphasized in the visualization. For example, when a user selects a particular clinical finding, the timelapse generator 325 can generate an updated timelapse that highlights the selected condition while maintaining context from other findings. The timelapse generator 325 can apply visual emphasis techniques such as color coding, outlining, and/or opacity adjustments to draw attention to the selected finding(s).

In some embodiments, the timelapse generator 325 can retrieve user input to modify treatment scenarios within the visualization. For example, a user can select different treatment options and/or behavioral modifications to see predicted outcomes. The timelapse generator 325 can generate alternative timelapse sequences showing potential results for each selected treatment approach. Multiple scenarios can be displayed simultaneously for comparison purposes, in embodiments.

In some embodiments, the timelapse generator 325 can process user requests to change the imaging modality used for visualization. For example, a user can switch between different modalities such as photographs, x-rays, and/or intraoral scans or 3D models generated from intraoral scans while maintaining the same temporal sequence. The timelapse generator 325 can retrieve records corresponding to the newly selected imaging modality and regenerate the timelapse visualization accordingly.

In some embodiments, a user can adjust temporal parameters of the timelapse visualization. The timelapse generator 325 can receive input to modify playback speed, select specific time ranges, and/or focus on particular milestone events. The timelapse generator 325 can process these temporal adjustments and update the visualization to match user preferences, in embodiments.

In some embodiments, the timelapse generator 325 can receive filtering commands that allow a user to isolate specific anatomical regions and/or tooth positions. For example, a user can select an individual tooth or quadrant to create a focused visualization. The timelapse generator 325 can apply spatial filtering to generate targeted timelapse sequences while maintaining proper anatomical context.

In some embodiments, the timelapse generator 325 can process user input to incorporate or exclude predictive elements from the visualization. For example, a user can toggle between historical data only and extended predictions showing a predicted future state. The timelapse generator 325 can dynamically add or remove predictive frames based on the user's selection.

In some embodiments, the timelapse generator 325 can transmit the modified visualization to computing device 360 via the UI controller 330 for display in the user interface. The timelapse generator 325 can provide real-time or near real-time updates as the user interacts with visualization controls. Modified visualizations can maintain proper registration and normalization while reflecting user-specified parameters.

In some embodiments, the timelapse generator 325 can track the state of a patient's teeth and/or the condition of their oral health using milestones on a timeline. In some embodiments, the timelapse generator 325 can enable visualization of individual actions and/or combinations of events and their impact on overall oral health. The timelapse generation 325 can use milestone data (e.g., stored as patient data 356) to filter records based on individual items and/or combinations of items, adjusting the visualization to meet specific clinical needs. Each milestone can correspond to a specific point in time, and can be associated with a particular imaging modality (or modalities) and/or clinical finding(s).

As an illustrative example, the multimodal timelapse system 215 can create a 3D model of segmented teeth and the jaws using a combination of CBCT data and intraoral scan data for a patient. The multimodal timelapse system 215 can receive updated data for the patient (e.g., updated scan and/or photographs), and based on the updated data can determine the position and/or orientation of the teeth, and can project the position and/or orientation of the teeth onto the 3D model. The multimodal timelapse system 215 can use scan data 351 corresponding to different imaging modalities (and optionally processed by input preprocessing engine 312) to simulate the bone remodeling and/or soft tissue changes over time (e.g., position of gingival margins as indicated by the scan or photograph). The multimodal timelapse system 215 can then generate a timelapse visualization of the 3D model displaying the bone remodeling and/or soft tissue changes over time.

In some embodiments, the timelapse generator 325 can perform bone remodeling simulation from image data 351 from multiple imaging modalities. For example, the timelapse generator 325 can combine CBCT data with intraoral scan data to create a 3D model of segmented teeth and jaw structures. The timelapse generator 325 can simulate bone remodeling and soft tissue changes over time based on tooth position and orientation data from updated scans or photographs in some embodiments.

In some embodiments, the bone remodeling simulation can show how bone structure adapts in response to tooth movement or orthodontic treatment. The timelapse generator 325 can predict bone adaptation patterns and generate updated 3D models showing bone remodeling progression over time. In some embodiments, the timelapse generator 325 can use virtual articulator data and/or occlusogram information to simulate forces acting on teeth and predict root dehiscences or fenestrations. The timelapse generator 325 can model parafunctional forces and their effects on bone structure. The simulation may show how changes in occlusion affect periodontal health and bone density over time.

In some embodiments, the bone remodeling simulation may be displayed as part of the timelapse visualization, showing the evolution of both hard and soft tissues. The timelapse generator 325 can project bone changes onto profile view images to demonstrate facial changes resulting from orthodontic treatment. This simulation capability can enhance treatment planning and patient education by showing predicted bone adaptation outcomes.

As another illustrative example, the multimodal timelapse system 215 can receive data from a virtual articulator and/or occlusogram to simulate forces (e.g., including parafunctional) on the patient's teeth and to predict root dehiscence/fenestrations. The multimodal timelapse system 215 can use CBCT data (in additional to other imaging modality data) to generate a multimodal timelapse visualization displaying how the patient's face changes over time (e.g., by displaying a profile view image of the patient). For example, the multimodal timelapse system 215 can take scan data, including a video of movements (e.g., protrusion, retrusion, lateral, excursive movements), and project the movements back onto a virtual articulator for range of motion.

As another illustrative example, the multimodal timelapse system 215 can provide virtual care by using photograph data captured by a patient's user device and creating positive motivational videos of improvements using a multimodal timelapse visualization. Alternatively, the multimodal timelapse system 215 can create a multimodal timelapse visualization that displays a negative outcome, e.g., if no treatment is accepted. In some embodiments, the multimodal timelapse system 215 can enable a user to share the generated timelapse visualizations, e.g., via social media, email, text, etc.

In some embodiments, user interface (UI) controller 330 can provide the timelapse visualizations (e.g., generated by timelapse generator 325) to a user device (e.g., computing device 360) for presentation in a user interface. In some embodiments, UI controller 330 can enable a user to interact with the timelapse visualization, e.g., by selecting certain clinical finding(s) to include or exclude, by selecting certain treatment(s) or behavioral modification(s) to include or exclude, by changing imaging modalities, by changing the point in time, by incorporating a timeline, etc. In some embodiments, the UI 330 can present milestones (e.g., as stored in patient data 356) as navigable elements within the timelapse visualization. In some embodiments, a user can select a specific milestone to view the patient's condition at that point in time. In some embodiments, the multimodal timelapse system 215 can display the progression between milestones, showing how treatments or lack of treatment affected the patient's oral health over time.

In some embodiments, the UI controller 330 can enable a user to select a clinical finding to include in the timelapse visualization, and cause the timelapse generator 325 to generate an updated timelapse visualization that corresponds to the user selection(s). In some embodiments, the user's selection of a particular clinical finding may not cause the timelapse generator 325 to ignore the unselected findings, but rather may generate a timelapse that focuses on the selected finding. That is, the affect of the unselected findings may still be incorporated in the timelapse, but may not be the focus of the timelapse visualization.

In some embodiments, computing device(s) 360 can be or include a user device. In some embodiments, the user device 360 can be used by a dental professional (e.g., a doctor, a dentist, a hygienist, and/or a technician) to educate a patient regarding the patient's oral health. The user device 360 can include a user interface (UI) to display the generated multimodal timelapse visualization, and optionally include tools to interact with and/or modify the visualization.

In some embodiments, UI controller 330 can enable a user to switch between different imaging modalities during presentation of the timelapse visualization. For example, the user can select a different imaging modality from the plurality of imaging modalities through interactive controls in the user interface. The multimodal timelapse system 215 can receive the user's modality selection and process the request to generate an updated visualization. In some embodiments, the timelapse generator 325 can retrieve records corresponding to the newly selected imaging modality from the timelapse data store 344. The timelapse generator 325 can apply normalization and registration processes to the records of the selected modality, in embodiments. The timelapse generator 325 can generate a new timelapse visualization representing the same temporal sequence in the selected imaging modality. The updated visualization can maintain the same chronological progression while displaying the clinical findings in the newly selected modality. In some embodiments, the UI controller 330 can provide the updated timelapse visualization to the user device for presentation. In some embodiments, this modality switching can occur seamlessly without requiring the user to restart the visualization process. In some embodiments, the user can switch between modalities multiple times to compare how the same clinical findings appear across different imaging types.

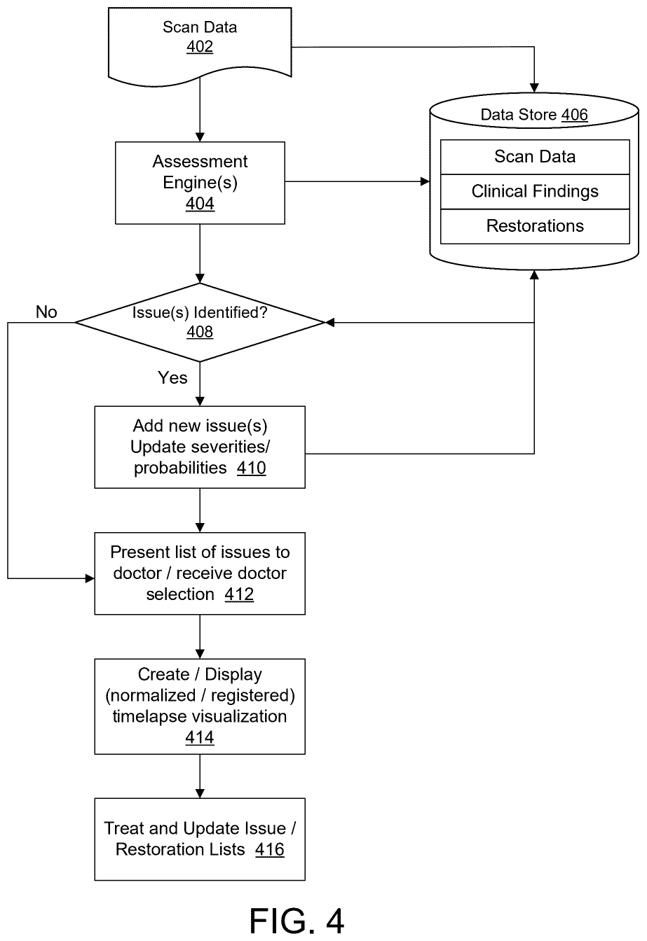

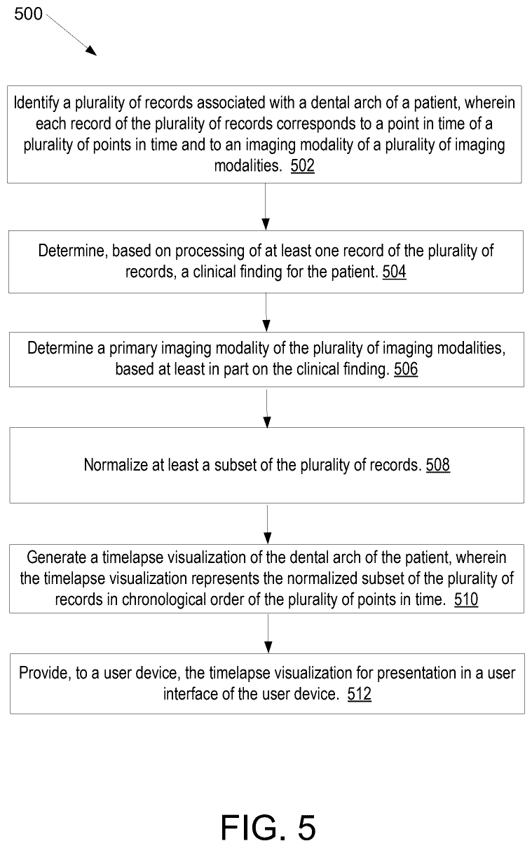

FIG. 4 illustrates a system workflow 400 for generating a multimodal timelapse, in accordance with some embodiments of the present disclosure. FIG. 5 illustrates a flow diagram of an example method 500 for generating and displaying a timelapse visualization of a dental arch of a patient, in accordance with some embodiments of the present disclosure. One or more of methods 400-500 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, one or more of methods 400-500 may be performed by the processing devices and the associated algorithms, e.g., as described in conjunction with FIG. 3. In embodiments, one or more of methods 400-500 is performed by processing logic comprising hardware, software, firmware, or a combination thereof. In certain embodiments, one or more of methods 400-500 may be performed by a single processing thread. Alternatively, one or more of methods 400-500 may be performed by two or more processing threads, each thread executing one or more individual functions, routines, subroutines, or operations of the method. In an illustrative example, the processing threads implementing one or more of methods 400-500 may be synchronized (e.g., using semaphores, critical sections, and/or other thread synchronization mechanisms). Alternatively, the processing threads implementing one or more of methods 400-500 may be executed asynchronously with respect to each other. Therefore, while FIGS. 4-5 and the associated descriptions list the operations of methods 400-500 in a certain order, in some embodiments, at least some of the described operations may be performed in parallel and/or in a different order. In some embodiments one or more operations of one or more of methods 400-500 is not performed.

Referring to FIG. 4, at block 402, the multimodal timelapse system 215 can receive image data, e.g., from a sensor (e.g., scanner, x-ray, CBCT, etc.). For example, the multimodal timelapse system 215 can receive new input files from a the oral state capture system(s) 310 of FIG. 3. The multimodal timelapse system 215 can store the received image data in data store 406. At block 404, the inputs can be processed by one or more assessment engine(s). In some embodiments, the assessment engine can be specific to the sensor type or imaging modality. That is, one assessment engine can process input generated by an intraoral scanner, one assessment engine can process input generated by a CBCT device, one assessment engine can process input generated by an x-ray machine, etc. In some embodiments, one assessment engine can process input generated by multiple input sources (e.g., can process images of multiple imaging modalities).

In some embodiments, the assessment engine(s) at block 404 can include one or more processing modules that analyze scan data 402 to identify clinical findings and/or oral health conditions. Each assessment engine can be configured to process specific types of imaging data and/or detect particular clinical conditions.

In some embodiments, the assessment engine(s) can include a caries detection engine that receives radiographic data as input. The caries detection engine can process x-ray images using machine learning algorithms trained to identify tooth decay patterns. The engine can output indications of caries presence with associated confidence levels and location coordinates within the dental arch.

In some embodiments, the assessment engine(s) can include a tooth wear analysis engine that receives intraoral scan data as input. The tooth wear analysis engine can perform geometric processing to compare tooth surfaces against normalized tooth shapes. The engine can calculate wear patterns and progression rates. The tooth wear analysis can output measurements of enamel loss and/or wear severity classifications, in embodiments.

In some embodiments, the assessment engine(s) can include a periodontal assessment engine that processes photographic data and/or intraoral scan data. The periodontal assessment engine can analyze gingival margins, tissue color, and/or pocket depth measurements. The periodontal assessment engine can apply segmentation algorithms to isolate gum tissue regions, in embodiments. The periodontal assessment engine can output indicators of gingivitis, periodontitis, and/or gingival recession with severity ratings.

In some embodiments, the assessment engine(s) can include a malocclusion detection engine that receives CBCT scan and/or intraoral scan data as input. The malocclusion detection engine can analyze tooth positioning, bite relationships, and/or jaw alignment. The malocclusion detection engine can compare patient anatomy against standard occlusal parameters, in embodiments. The malocclusion detection engine can output classifications of bite irregularities and/or alignment deviations.

In some embodiments, the assessment engine(s) can include sensor data processing engines that receive input from multisensory tools such as electronic compliance indicators. The sensor data processing engines can process pressure, temperature, and/or accelerometer data to detect bruxism events and/or compliance patterns. The sensor processing engines can output behavioral analysis results and/or force measurement data.

In some embodiments, the assessment engine(s) can correspond to clinical finding identification engine 320 of FIG. 3. The output of the assessment engine(s) can be stored in data store 406, which can correspond to timelapse data store 344 of FIG. 3.

In some embodiments, the assessment engine(s) at block 404 can output clinical findings, and the multimodal timelapse system 215 can store the output of the assessment engine(s) in data store 406 (e.g., as clinical findings). In some embodiments, data store 406 can store an issues list, which is a list of existing clinical findings for a patient. Each clinical finding in the issues list can be associated with a corresponding severity and/or probability value.

In some embodiments, the issues list can be a record of all clinical findings identified for a specific patient. The issues list can corresponds to clinical findings data 355 of FIG. 3. Each entry in the issues list can represent a distinct clinical condition such as caries, periodontal disease, tooth wear, malocclusion, or any other condition detected through assessment engine processing. In some embodiments, each clinical finding entry in the issues list can include associated metadata that characterizes the condition's current state or state associated with a particular point in time. The severity parameter can indicate the extent or progression of the clinical finding using standardized classification systems. The probability value can represent the diagnostic confidence level for that particular finding based on the assessment engine's analysis. In some embodiments, the issues list can include temporal information indicating when each clinical finding was first detected and last updated. In some embodiments, the issues list can track changes in severity and probability values over time as new assessment data becomes available.

In some embodiments, data store 406 can store a list of restorations, which can include a record of dental restorative work completed for a patient. In some embodiments, each restoration entry can include information about the type of restoration performed, such as fillings, crowns, bridges, implants, and so on. In some embodiments, the restoration data can specify the anatomical location within the dental arch where the restoration was placed. In some embodiments, the restoration data can include temporal information indicating when each restorative procedure began and/or was completed. In some embodiments, the restoration records can track the materials used and the treating practitioner who performed the work. In some embodiments, each restoration entry can include quality assessments and expected longevity data based on the restoration type and patient factors.

In some embodiments, the restorations list can be linked to corresponding entries in the issues list to establish treatment relationships. In some embodiments, a restoration entry can indicate that a previous clinical finding such as caries has been addressed through treatment. In some embodiments, the multimodal timelapse system 215 can update both the issues list and restorations when treatment is completed to reflect the changed clinical status.

In some embodiments, the restorations list can influence future assessment and prediction algorithms. In some embodiments, restored locations can be flagged for different types of monitoring, such as secondary caries detection around existing fillings. In some embodiments, the restoration data can inform treatment planning by identifying areas that require special consideration due to previous dental work. The restorations list can corresponds to treatment data 353 of FIG. 3.

At block 408, the multimodal timelapse system 215 can determine whether an assessment (e.g., determined at block 404) is indicative of an issue (e.g., an oral health concern). The assessment is determined to be indicative of an issue when the corresponding clinical finding exceeds a predetermined threshold criterion. The multimodal timelapse system 215 can compare assessment results against established diagnostic parameters for various oral health conditions. For example, caries detection algorithms can identify cavities when radiograph density measurements fall below normal tooth structure values.

In some embodiments, the multimodal timelapse system 215 can use confidence scores generated by one or more AI models of the assessment engines to determine the significance of the assessment result(s). An assessment result with a confidence level above a predetermined threshold can be classified as indicative of a clinical issue. In some embodiments, the multimodal timelapse system 215 can use confidence scores of 70%, 80%, or 90% depending on the specific clinical finding and/or imaging modality used.

In some embodiments, the multimodal timelapse system 215 can evaluate multiple assessment criteria simultaneously to improve diagnostic accuracy. For example, geographic analysis of tooth wear can be combined with surface texture analysis to confirm wear patterns. As another example, periodontal assessments can incorporate gingival margin measurements, pocket depth measurements, and/or tissue color analysis to identify disease indicators.

In some embodiments, the multimodal timelapse system 215 can compare current assessment results with historical patient data to identify progressive changes. Measurements that show significant deviation from previous baseline values (e.g., deviation above a threshold) can indicate developing issues. The multimodal timelapse system 215 can track rate of change parameters to distinguish between normal variation and pathological progression.

In some embodiments, the assessment may be considered indicative of an issue when multiple imaging modalities provide corroborating evidence. Cross-modal validation can increase diagnostic confidence and/or reduce false positive identifications. In some embodiments, the multimodal timelapse system 215 can rely on agreement between two or more assessment engines before classifying a finding as a clinical issue.

If the assessment is indicative of an issue, at block 410, the multimodal timelapse system 215 can determine whether the issue is a known issue, e.g. by performing issue classification and/or tracking operations. For example, the multimodal timelapse system 215 can compare the identified issue to the issues list stored at data store 406. In some embodiments, the issues list can correspond to clinical findings data 355 and/or patient data 356 of FIG. 3. This comparison can determine whether the identified issue represents a new clinical finding or an update to a previously identified clinical finding.

If the issue is included in the issues list, the multimodal timelapse system 215 can determine that it is a known issue. In some embodiments, the multimodal timelapse system 215 can update the probability and/or severity parameters associated with that issue in the issues list. These updates can reflect disease progression, treatment response, and/or changes in diagnostic confidence based on additional imaging data.

If the issue is not included in the issues list, the multimodal timelapse system 215 can determine that it is not a known issue (e.g., that it is a new issue). In some embodiments, the multimodal timelapse system 215 can add the identified issue to the issues list. In some embodiments, the multimodal timelapse system 215 can add the newly identified issue to the issues list with initial probability and/or severity values. The new entry can include metadata such as detection date, imaging modality (or modalities) used for identification, and/or anatomical location within the dental arch.

In some embodiments, the multimodal timelapse system 215 can correlate multiple assessment results to determine if separate findings represent different aspects of the same underlying condition. For example, gingival recession and root caries detected in the same anatomical region can be linked as related clinical findings. The multimodal timelapse system 215 can establish causal relationships between issues to improve treatment planning accuracy.

In some embodiments, the updated issues list can serve as input for subsequent processing steps including, for example, treatment option identification and/or timelapse visualization generation. The probability and/or severity data associated with each issue can influence the prioritization of clinical findings presented to dental practitioners and the emphasis given to specific conditions in patient visualizations.

If no assessment (e.g., determined at block 404) is indicative of an issue, the multimodal timelapse system 215 can proceed to block 412.

At block 412, the multimodal timelapse system 215 can present the list of issues to a dental practitioner (e.g., to a user of computing device 360). In some embodiments, the multimodal timelapse system 215 can generate a presentation interface that displays identified clinical findings for practitioner review. In some embodiments, the multimodal timelapse system 215 can organize the issues list by severity level, anatomical location, and/or detection confidence, e.g., to facilitate clinical decision-making. In some embodiments, each issue entry can be presented with associated metadata including detection date, imaging modality used, and/or probability scores.

In some embodiments, the multimodal timelapse system 215 can enable user interaction, e.g., by allowing the user (e.g., the dental practitioner) to select one (or more) issues to discuss with the patient. For example, a doctor can interact with the multimodal timelapse system 215 by reviewing the list of issues and selecting one (or more) issues to discuss with the patient. In some embodiments, the presentation interface can enable interactive selection of individual clinical findings from the issues list. For example, the dental practitioner can click or select specific issues to view detailed information about each condition. In some embodiments, the system can provide visual indicators such as color coding or icons to distinguish between different types of clinical findings.

In some embodiments, the multimodal timelapse system 215 can present filtering options that allow a practitioner to focus on specific categories of issues. For example, the practitioner can filter the display to show only new findings, high-severity conditions, and/or issues requiring immediate attention. In some embodiments, the multimodal timelapse system 215 can group related clinical findings together to show comprehensive treatment needs. In some embodiments, the presentation can include summary statistics showing, for example, the total number of issues, distribution by condition type, and/or overall oral health status indicators. In some embodiments, the multimodal timelapse system 215 can highlight issues that have changed in severity or probability since the last assessment. In some embodiments, the interface can provide direct access to underlying imaging data and assessment results that support each clinical finding.

In some embodiments, the selection from the user can trigger the multimodal timelapse system 215 to, at block 414, retrieve the relevant records from the data store, normalize the records, register the records, and create a multimodal timelapse visualization, e. g, as described with respect to the multimodal timelapse system 215 of FIG. 3 In some embodiments, the records can be normalized and/or registered at previous step (e.g., block 410 or 412), to reduce latency experienced by the user in the generation of the timelapse. The generated timelapse visualization can be shown to the patient, and the patient and dental practitioner can have a discussion about potential treatments, behavioral modifications, outcomes, etc. In some embodiments, a user (e.g., a dental practitioner) can modify the selections of issues and/or treatments, and the multimodal timelapse system 215 can generate updated timelapse visualizations corresponding to the updated selections.

At block 416, based on a treatment (e.g., provided by the user via the UI), the multimodal timelapse system 215 can update the issues list and/or restorations in the data store 406. In some embodiments, if the treatment is a restoration, the multimodal timelapse system 215 can update the issues list and the restorations list to indicate that there is no longer an old issue. The multimodal timelapse system 215 can further indicate that there may be a need to treat the location of the restoration differently after it has been restored (e.g., secondary caries).

In some embodiments, at block 416, the multimodal timelapse system 215 can update patient records based on completed dental treatments. In some embodiments, the multimodal timelapse system 215 can receive treatment information indicating that a restoration procedure has been performed on the patient. In some embodiments, the system can modify the issues list in datastore 406 to reflect that a previously identified clinical finding has been addressed through treatment.

In some embodiments, the multimodal timelapse system 215 can add new entries to the restorations list of data store 406 when dental work is completed. In some embodiments, each restoration entry can include information about the type of procedure performed, the anatomical location, and the completion date. In some embodiments, the system can link the new restoration entry to the corresponding issue that was treated.

In some embodiments, the multimodal timelapse system 215 can update the severity and probability parameters for treated conditions in the issues list. In some embodiments, a successfully treated caries can be marked as resolved while maintaining the historical record of the original finding. In some embodiments, the system can flag restored locations for different types of future monitoring, such as secondary caries detection around existing fillings.

In some embodiments, the multimodal timelapse system 215 can establish relationships between completed restorations and potential future issues. In some embodiments, the multimodal timelapse system 215 can indicate that restored areas require special consideration during subsequent assessments. In some embodiments, the updated restoration and issue data can influence future timelapse visualizations and treatment planning recommendations. For instance, in some embodiments, the multimodal timelapse system 215 can adjust subsequent timelapse sequences to start from the restored state rather than the original diseased condition. In some embodiments, restored locations can be flagged for specialized monitoring that focuses on secondary conditions such as recurrent caries around existing fillings.

In some embodiments, the multimodal timelapse system 215 can generate updated patient records that reflect the current clinical status following treatment completion. In some embodiments, these updated records can serve as baseline data for future assessment cycles and timelapse generation processes. For example, in some embodiments, the restoration data can influence how assessment engines 404 analyze future scan data 402. In some embodiments, the multimodal timelapse system 215 can apply different diagnostic criteria and threshold values for restored areas compared to natural tooth structures. In some embodiments, the assessment engines 404 can modify their analysis algorithms to account for the presence of dental restorations when identifying new clinical findings.

Referring to FIG. 5, at block 502, processing logic identifies a plurality of records associated with a dental arch of a patient. Each record corresponds to a point in time and to an imaging modality of a plurality of imaging modalities. In some embodiments, processing logic can query a data store (e.g., data store 308 of FIG. 3) to retrieve imaging records for the patient. In some embodiments, the plurality of imaging modalities can include, for example, intraoral scan, extraoral scan, near-infrared image, cone beam computed tomography (CBCT), photograph, video, or radiograph.

At block 504, based on processing of at least one record, processing logic determines a clinical finding for the patient. In some embodiments, processing logic can utilize the clinical finding identification engine 320 to analyze the plurality of records and identify conditions such as caries, peridonal disease, tooth wear, malocclusion, and/or other health issues. The clinical finding degermation can involve automated assessment algorithms that process geometric features, density patters, color variations, and/or other characteristics specific to each imaging modality.

In some embodiments, processing logic can provide, as input, the at least one record to an AI model that is trained to provide an indication of the clinical finding. Processing logic can receive, as output from the AI model, the indication of the clinical finding. In some embodiments, the clinical finding can be, for example, an indication of caries, malocclusion, periodontal disease, tooth wear, gingival recession, tooth fracture, bruxism, temporomandibular joint disorder, structural anomaly, and/or restorative disorder.

In some embodiments, processing logic can provide, as input, one or more of the plurality of records to an AI model trained to provide an indication of the clinical finding. Processing logic can receive, as output from the AI model, a plurality of indications of the clinical finding. Each indication can correspond to a record, and each indication can include a confidence level. Processing logic can identify the clinical finding with the highest confidence level. In some embodiments, processing logic can provide, to the user device, each indication and its corresponding confidence level.

In some embodiments, processing logic can compare the at least one record to a corresponding predetermined criterion to determine the clinical finding. In some embodiments, the predetermined criterion can include threshold values for specific clinical conditions based on established diagnostic parameters. In some embodiments, processing logic can compare geometric measurements from tooth wear analysis against normal tooth surface profiles to identify excessive wear patterns. In some embodiments, the predetermined criterion can include radiographic density thresholds that indicate the presence of caries when tooth structure density falls below normal values.

In some embodiments, the comparison process can involve evaluating multiple parameters simultaneously to improve diagnostic accuracy. In some embodiments, the predetermined criterion can include pocket depth measurements for periodontal disease detection or gingival margin position thresholds for recession identification. In some embodiments, processing logic can use standardized clinical classification systems to establish the predetermined criteria for different oral health conditions.

In some embodiments, the predetermined criterion can be specific to the imaging modality being analyzed. For example, photographic records can be compared against color and texture criteria for soft tissue conditions, while CBCT data can be evaluated against bone density and structural parameters. In some embodiments, processing logic can apply different threshold values based on patient demographics, age, and/or medical history to customize the predetermined criteria for individual cases.

At block 506, processing logic determines, based at least in part on the clinical finding, a primary imaging modality of the plurality of imaging modalities. Processing logic can select the imaging modality that provides the most accurate visualization or diagnostic information for the clinical finding identified at operation 504. For example, x-ray imaging can be selected as the primary modality for caries visualization, while photographic imaging can be selected for gingival recession or aesthetic concerns. The selection process can consider factors such as diagnostic accuracy, patient comprehension, and/or visualization clarity for the specific condition.

In some embodiments, the primary imaging modality that corresponds to the clinical finding is determined based on a ranking of the plurality of imaging modalities. In some embodiments, the ranking of the plurality of imaging modalities can be based on the diagnostic accuracy of each modality for specific clinical findings. For example, radiographic imaging can be ranked highest for caries detection due to its ability to visualize subsurface tooth decay. Photographic imaging can be ranked highest for gingival conditions because it provides clear visualization of soft tissue color and texture changes. CBCT imaging can be ranked highest for bone-related findings such as root resorption or periodontal bone loss. Intraoral scan data can be ranked highest for tooth wear analysis due to its precise geometric measurements of surface changes. Near-infrared imaging can be ranked highest for early decalcification detection before cavitation occurs.

In some embodiments, the ranking can consider the correlation between each imaging modality and the specific clinical finding being evaluated. In some embodiments, certain imaging modalities can have higher accuracy rates for particular oral health conditions based on their physical imaging principles. In some embodiments, processing logic can select the imaging modality with the highest ranking score for the identified clinical finding as the primary modality for timelapse visualization.

In some embodiments, the ranking can be determined through statistical analysis of diagnostic performance across large datasets. In some embodiments, machine learning algorithms can establish ranking hierarchies based on sensitivity and specificity measurements for each modality-condition pairing. In some embodiments, the ranking system can be updated over time as new imaging technologies become available or as diagnostic accuracy improves.

In some embodiments, the at least one record that is processed to determine the clinical finding has a first imaging modality, and the primary imaging modality is a second imaging modality that is different from the first imaging modality. In some embodiments, the multimodal timelapse system 215 can combine the clinical accuracy of specialized imaging modalities with the visual clarity of patient-friendly modalities. In some embodiments, this approach can enhance both diagnostic precision and patient comprehension within the same clinical workflow. For example, processing logic can process x-ray data to detect caries while the primary imaging modality selected for timelapse visualization can be photographic data for enhanced patient understanding. As another example, processing logic can identify periodontal disease using CBCT scan data and generate the timelapse visualization using intraoral photographs to show visible gum changes over time.

In some embodiments, this cross-modal approach can leverage the diagnostic accuracy of one imaging modality while optimizing patient communication through a different modality. For example, radiographic data can provide superior clinical detection capabilities for subsurface conditions, but photographic data can offer more intuitive visualization for patient education purposes. As another example, processing logic can use near-infrared imaging to detect early decalcification that is not visible in standard photographs and can create predictive photographic sequences showing how the detected decalcification might progress to visible cavitation over time.

At block 508, processing logic normalizes at least a subset of the plurality of records. In some embodiments, the subset corresponds to the primary imaging modality. In some embodiments, processing logic performs the normalization across the plurality of records for a size, a scale, a color balance, a brightness, lighting conditions, contrast levels, magnification factor, capture angle, and/or an orientation. That is, the processing logic can address variations in lighting conditions, color balance, contrast levels, magnification factors, size, scale, capture angle, and/or orientation that may exist between imaging sessions. In some embodiments, the normalization process can utilize the normalization data 357 stored in the timelapse data store 344 of FIG. 3 to apply consistent transformation parameters across the subset of records.

In some embodiments, the normalization process can be performed on at least a subset of the plurality of records that corresponds to the primary imaging modality. The system can normalize records within the same imaging modality to establish consistent visual parameters such as size, scale, brightness, color balance, and/or orientation. The normalization can address variations that occur between imaging sessions using the same modality type, such as differences in lighting conditions for photographs or exposure settings for radiographs.

In some embodiments, the processing logic can perform normalization across different imaging modalities. Cross-modality normalization can enable the integration of data from different imaging sources into a unified visualization while maintaining spatial and temporal consistency. The processing logic can apply modality-specific normalization parameters so that records from different imaging types can be accurately combined within the same timelapse sequence.

In some embodiments, the processing logic can perform registration operations within the same imaging modality and/or across different imaging modalities. Registration within the same imaging modality can align records captured at different time points to account for variations in patient positioning, camera angles, and/or imaging device placement. The processing logic can compute transformation parameters to spatially align images of the same modality type for accurate temporal comparison. In some embodiments, cross-modal registration can establish spatial correspondence between records from different imaging modalities captured at the same or different time points. For example, the processing logic can identify anatomical landmarks that are visible across multiple imaging modalities to create transformation matrices that map spatial coordinates between different modality types. The cross-modal registration can enable the system to correlate clinical findings detected in one imaging modality with corresponding anatomical regions in other modalities. In some embodiments, the registration process can be performed before normalization to ensure that spatial alignment is established prior to visual parameter adjustments.

In some embodiments, the subset of the plurality of records includes a first record corresponding to a first point in time of the plurality of points in time and to a first imaging modality of the plurality of imaging modalities. The subset of the plurality of records includes a second record corresponding to a second point in time of the plurality of points in time and to a second imaging modality of the plurality of imaging modalities. The timelapse visualization represents at least one of the first imaging modality or the second imaging modality. That is, the timelapse visualization can be generated in one of the first imaging modality or the second imaging modality. In some embodiments, the timelapse visualization can be generated in both the first imaging modality and the second imaging modality.

At block 510, processing logic generates a timelapse visualization of the dental arch of the patient. The timelapse visualization represents the normalized subset of the plurality of records in chronological order. In some embodiments, the timelapse visualization of the dental arch is presented in a dental chart format. In some embodiments, the timelapse visualization is a animated represented of the normalized subset of the plurality of records. In some embodiments, the timelapse generation can be performed by timelapse generator 325 of FIG. 3, which can create smooth transitions between time points through interpolation techniques and/or registration processes. The timelapse visualization can demonstrate the progression of the clinical finding over time, showing how conditions have developed or changes across the temporal sequence.

In some embodiments, processing logic can generate, for one or more pairs of chronologically consecutive records in the subset of the plurality of records, one or more intermediary images. Processing logic can insert, for each of the one or more pairs, the one or more intermediary images in between corresponding pair of chronologically consecutive records. In some embodiments, the one or more intermediary images are generated using interpolation or optical flow techniques.

In some embodiments, the timelapse visualization includes data that corresponds to at least one imaging modality other than the primary imaging modality. For example, in some embodiments, the timelapse visualization can incorporate CBCT data to provide subsurface bone structure information while using photographs as the primary imaging modality for patient-friendly visualization. As another example, in some embodiments, the timelapse generator 325 can overlay x-ray findings onto intraoral scan sequences to show both surface and subsurface changes simultaneously. As another example, in some embodiments, the timelapse generator 325 can combine near-infrared imaging data with photographic sequences to highlight decalcification patterns that are not visible in standard photographs. In some embodiments, the multimodal approach can enhance diagnostic accuracy by correlating findings across different imaging types while maintaining optimal visualization in the primary modality. In some embodiments, the timelapse visualization can fade between different imaging modalities within the same timelapse sequence to demonstrate how skeletal changes relate to soft tissue modifications over time.

In some embodiments, the multimodal data integration synthesizes information from disparate imaging modalities (e.g., disparate imaging sources) into a unified timelapse presentation. In some embodiments, the timelapse generator 125 can process imaging data from multiple distinct modalities captured at different time points and combine this heterogeneous data into a cohesive temporal narrative. For example, each imaging modality can contribute unique diagnostic information that, when integrated, creates a more comprehensive understanding of oral health progression than any single modality could provide alone.

In some embodiments, the processing logic can perform cross-modal data fusion by identifying corresponding anatomical features across different imaging types and temporally aligning these features within the timelapse visualization. The processing logic can establish spatial correspondence between modalities using anatomical landmarks, and can overlay and/or blend information from different imaging modalities. In some embodiments, the processing logic can weight the contribution of each modality based on its diagnostic relevance to the identified clinical finding in order to feature (or highlight) clinically significant information in the timelapse visualization.

In some embodiments, the multimodal integration can reveal temporal relationships between different tissue types and/or anatomical structures that may not be apparent when viewing individual modalities in isolation. The timelapse visualization can demonstrate how changes detected in one imaging modality correlate with or predict changes visible in another modality over time. The processing logic can use this cross-modal temporal correlation to enhance predictive accuracy and provide more comprehensive patient education by showing the interconnected nature of oral health conditions across different tissue types and/or anatomical regions.

In some embodiments, the processing logic can dynamically adjust the opacity, color mapping, and/or visual emphasis of different modalities within the same visualization frame to highlight specific clinical findings and/or temporal changes. For example, the processing logic can create layered visualizations where subsurface information from radiographic and/or CBCT data provides context for surface changes visible in photographic and/or intraoral scan data. This layered approach can enable practitioners to demonstrate the underlying causes of visible symptoms and help patients understand the relationship between internal pathology and external manifestations of oral health conditions, in embodiments.

In some embodiments, the timelapse visualization can highlight a region (or multiple regions) within the timelapse visualization that correspond to the clinical finding across multiple (or all) imaging modalities for the patient. In some embodiments, processing logic can identify an area of interest in at least one of the plurality of imaging modalities, the area of interest corresponding to the clinical finding. The processing logic can provide, in the timelapse visualization, a visual indicator corresponding to the area of interest in the at least one of the plurality of imaging modalities other than the primary imaging modality, wherein the visual indicator highlights the area of interest. As an illustrative example, the visual indicator can be a color coding, outlining, and/or opacity adjustment to draw attention to the region of clinical interest. In some embodiments, the processing logic can correlate clinical findings detected in different imaging modalities and emphasize the same anatomical locations within the primary imaging modality visualization.

In some embodiments, processing logic can identify a clinical finding for a particular anatomical region of the dental arch. The processing logic can map the clinical finding to corresponding spatial coordinates across different imaging modalities (e.g., using registration data 354 and/or transformation parameters). The processing logic can then include, in the timelapse visualization, a visual indicator (e.g., a highlighting) of the clinical finding across multiple imaging modalities to maintain consistent emphasis on affected areas throughout the timelapse progression. In some embodiments, the highlighting techniques can be modality-specific, such as intensity adjustments for radiographic data and color overlays for photographic data. The highlighted region(s) can persist across time points within the timelapse visualization to demonstrate the progression or improvement of clinical findings in the same anatomical locations displayed using multiple imaging modalities.

At block 512, processing logic provides, to a user device (e.g., computing device 360 of FIG. 3), the timelapse visualization for presentation in a user interface of the user device. In some embodiments, UI controller 330 of FIG. 3 can transmit the visualization data through network 350 to compute device 360 and/or other user device(s) for display to a partitioner and/or patient. In some embodiments, the UI may include controls for playback speed, timeline navigation, and/or filtering options that allow a user to focus on specific aspects of the visualization.

In some embodiments, the multimodal timelapse system can generate a visualization that displays the future state of the patient's teeth if the patient accepts treatment. Processing logic can identify a treatment corresponding to the clinical finding. Examples of a treatment include orthodontic treatment, retainer realignment treatment, restorative treatment, periodontal treatment, behavioral modification, appliance-based treatment, interdisciplinary treatment, and so on. Processing logic can generate one or more predicted records representing the dental arch of the patient affected by the treatment at a future point in time. Processing logic can update the visualization of the dental arch of the patient to include the one or more predicted records, and can provide, to the user device, the updated timelapse visualization for presentation in the UI of the user device. In some embodiments, playback of the updated timelapse visualization can be simultaneous to playback of the timelapse visualization generated at operation 510. In some embodiments, the one or more predicted records can correspond to the primary imaging modality.

In some embodiments, in generating the one or more predicted records, processing logic can determine an effect of the treatment on a condition of at least a portion of the dental arch of the patient at the future point in time, and can apply the effect to the most recent record of the plurality of records. In some embodiments, the effect is determined using artificial intelligence, and/or using a predetermined set of rules.

In some embodiments, the multimodal timelapse system can provide virtual care for a patient, e.g., by taking using photographs to create a positive motivational video of improvements with the timelapse visualization. Processing logic can identify one or more photographs of the patient (e.g., the photographs can include the mouth of the patient, the teeth, etc.). Processing logic can generate one or more predicted photographs of the patient. The one or more predicted photographs can represent an improvement of the state of the mouth of the patient, e.g., affected by the treatment, at corresponding future points in time. Processing logic can generate, based on the one or more predicted photographs of the patient, a video representing the improvement of state of the mouth of the patient over time.

In some embodiments, an improvement of the mouth can refer to positive change(s) in the patient's oral health status and/or aesthetic appearance during and/or following treatment intervention. For example, the improvement can include restoration of a damaged tooth structure through fillings, crowns, and/or other restorative procedures. As another example, the improvement can encompass healing of gingival tissues resulting in reduced inflammation, decreased pocket depths, and/or improved tissue color. In some embodiments, the improvement can include orthodontic corrections that align teeth into position and/or improve bite relationship. In some embodiments, the improvement can involve enhanced facial aesthetics through changes in facial profile. In some embodiments, improvement can include resolution of functional issues such as improved chewing efficiency or reduced jaw joint discomfort.

In some embodiments, the improvement can be visualized through predicted photographs showing the expected positive outcomes of proposed treatments. In some embodiments, improvement can include correction of spacing issues, replacement of missing teeth with implants or prosthetics, and so on. In some embodiments, the system can generate timelapse visualizations demonstrating the progressive improvement that occurs during and/or after treatment completion

In some embodiments, the multimodal timelapse system can generate a visualization that displays the future state of the patient's teeth if the patient does not accept treatment. Processing logic can generate one or more predicted records representing the dental arch of the patient affected by the clinical finding at a future point in time. Processing logic can update the timelapse visualization of the dental arch to include the one or more predicted records, and can provide, to the user device, the updated timelapse visualization for presenting in the UI. In some embodiments, the one or more predicted records can represent the dental arch of the patient affected by a secondary clinical finding at the future point in time. The secondary clinical finding can be associated with the clinical finding. For example, the clinical finding can be bruxism, which can affect the occlusal wear, gingival recession, tooth height, etc., of the patient's teeth. Hence, the one or more predicted records can represent the dental arch of the patient affected by occlusal wear, gingival recession, and/or reduced tooth height at the future point in time. In some embodiments, the one or more predicted records can correspond to the primary imaging modality (e.g., the predicted records can be generated to reflect the primary imaging modality).

In some embodiments, in generating the one or more predicted records, processing logic can determine an effect of the clinical finding on a condition of at least a portion of the dental arch of the patient at a future point in time, and can apply the effect to the most recent record of the plurality of records. In some embodiments, processing logic can identify the effect using artificial intelligence and/or a predetermined set of rules. In some embodiments, the effect is determined based on patient data, familial data, general population data, and/or statistical analysis associated with the clinical finding. In some embodiments, the effect can include a potential outcome, and/or a velocity of the progression of the clinical finding.

In some embodiments, the multimodal timelapse system can generate a video showing a negative outcome, e.g., if the patient refuses treatment. Processing logic can identify one or more photographs of the patient (e.g., the photographs can include the mouth or teeth of the patient). Processing logic can generate one or more predicted photographs of the patient. The one or more predicted photographs can represent a deterioration of the mouth/teeth of the patient affected by the clinical finding at a corresponding future point in time. Processing logic can generate, based on the one or more predicted photographs, a video representing the deteriorations of the mouth of the patient over time.

In some embodiments, the deterioration of the mouth can refer to the progressive worsening of oral health conditions affecting the patient's dental arch, teeth, and/or surrounding tissues. For example, the deterioration can include advancement of caries leading to increased cavity size and depth. In some embodiments, deterioration can encompass progression of periodontal disease resulting in increased gingival recession, pocket depth, and/or bone loss, for example. In some embodiments, deterioration of the mouth can include progressive tooth wear patterns that reduce tooth height and/or alter occlusal surfaces. In some embodiments, the deterioration can involve structural changes such as tooth fractures, enamel loss, and/or root surface exposure. In some embodiments, deterioration can include soft tissue changes such as increased inflammation, tissue discoloration, or loss of attached gingiva.

In some embodiments, the deterioration can result from untreated clinical findings that progress over time without intervention. In some embodiments, deterioration can be accelerated by patient behaviors such as poor oral hygiene, dietary habits, and/or parafunctional activities like bruxism. In some embodiments, the multimodal timelapse system can model and visualize this deterioration to demonstrate potential negative outcomes if clinical findings remain untreated. In some embodiments, deterioration of the mouth can be quantified through measurements of tissue loss, structural damage, and/or functional impairment. In some embodiments, the predicted deterioration can serve as a motivational tool for patient education and treatment acceptance.

In some embodiments, generating the one or more predicted records is performed based on the processing one or more of the plurality of records and sensor data corresponding to the patient. The sensor data can be generated by a multisensory tool, and can include, for example, pressure, temperature, and/or acceleration data.

In some embodiments, the multimodal timelapse system can account for discrepancies between imaging modalities. Processing logic can determine, based on the processing of the at least one record, a plurality of indications of the clinical finding for the patient. Each indication can correspond to an imaging modality. Processing logic can determine a discrepancy between at least two indications of the clinical finding. Processing logic can rank the at least two indications based on a correlation between the clinical finding and the corresponding imaging modality. Processing logic can resolve the discrepancy based on the ranking. For example, certain imaging modalities can have a higher accuracy for certain clinical findings, and processing logic can rank the indications based on the accuracy of the corresponding imaging modality.

In some embodiments, processing logic can determine a confidence for each of the at least two indications associated with the discrepancy. Processing logic can determine an overall confidence by combining the confidence for each of the at least two indications, and can provide, to the user device, the overall confidence corresponding to the clinical finding. In some embodiments, processing logic can identify the indication with the highest confidence as the indication of the clinical finding to resolve the discrepancy.

In some embodiments, the multimodal timelapse system can predict future states affected by multiple clinical findings. Processing logic can determine, based on the processing of the at least one record, a plurality of clinical findings for the patient. Processing logic can generate one or more predicted records representing the dental arch of the patient affected by the plurality of clinical findings at a future point in time. Processing logic can generate an updated timelapse visualization by updating the timelapse visualization to include the one or more predicted records, and can provide, to the user device, the updated timelapse visualization for presentation in the UI.

In some embodiments, the multimodal timelapse system can predict future states affected by a subset of the multiple clinical findings. For example, a user (e.g., a dentist) can select certain clinical findings to include in the predicted simulation, or certain clinical findings to exclude in the predicted simulation. Processing logic can receive, for the user device, a user interaction indicating a first clinical finding of the plurality of clinical findings. Processing logic can determine a subset of the clinical findings that includes the plurality of clinical findings other than the first clinical finding. Processing logic can generate a second set of one or more predicted records representing the dental arch of the patient affected by the subset of clinical findings at the future point in time. Processing logic can generate a second updated timelapse visualization of the dental arch by updating the timelapse visualization to include the second set of the one or more predicted records, and can provide, to the user device, the second updated timelapse visualization for presentation in the UI.

In some embodiments, the multimodal timelapse system can predict the future state affected by only one clinical finding (e.g., the user selected a single clinical finding to include in the predicted simulation). Processing logic can receive, from the user device, a user interaction indicating a first clinical finding of the plurality of clinical findings. Processing logic can generate a third set of one or more predicted records representing the dental arch of the patient affected by the first clinical finding at the future point in time. Processing logic can generate a third updated timelapse visualization of the dental arch by updating the timelapse visualization to include the third set of the one or more predicted records, and can provide, to the user device, the third updated timelapse visualization for presentation in the UI.

In some embodiments, the multimodal timelapse system can provide multiple timelapse visualizations, each one representing a different outcome (e.g., corresponding to different treatments, to no treatment, or to behavioral modifications, etc.). Processing logic can generate a first set of one or more predicted records representing the dental arch of the patient affected by the clinical finding at a future point in time. Processing logic can generate a second set of one or more predicted records representing the dental arch of the patient affected by behavior modifications corresponding to the clinical finding at the future point in time. Processing logic can generate a third set of one or more predicted records representing the dental arch of the patient affected by a first treatment corresponding to the clinical finding at the future point in time. Processing logic can generate a fourth set of one or more predicted records representing the dental arch of the patient affected by a second treatment corresponding to the clinical finding at the future point in time. Processing logic can generate a plurality of updated timelapse visualizations of the dental arch of the patient, wherein each updated timelapse visualization comprises the timelapse visualization updated to include one of the first set of one or more predicted records, the second set of one or more predicted records, the third set of one or more predicted records, or the fourth set of one or more predicted records. Processing logic can provide, to the user device, the plurality of updated timelapse visualizations for presentation in the user interface of the user device. In some embodiments, the plurality of updated timelapse visualizations can be presented in the UI simultaneously, e.g., to highlight the differences between treatments, or between a treatment and no treatment, etc.

In some embodiments, processing logic can identify a first record corresponding to a first imaging modality (e.g., a CBCT) at a first point in time. Processing logic can identify a second record corresponding to a second imaging modality (e.g., an intraoral scan) at the first point in time. Processing logic can identify, based on the first record and/or the second record, a position of one or more teeth and/or an orientation of the one or more teeth. Processing logic can generate, based on the first record and the second record, a 3D model of the dental arch of the patient. The 3D model can include the one or more teeth and the jaw.

In some embodiments, processing logic can receive a third record corresponding to a third imaging modality, and corresponding to a second point in time that follows, chronologically, the first point in time. Processing logic can identify a change in the position of the one or more teeth and/or in the orientation of the one or more teeth. The change can correspond to a movement of the jaw. Processing logic can generate, based on the change, an updated 3D model representing a simulation of the movement of the jaw over time.

In some embodiments, processing logic can receive a fourth record corresponding to a fourth imaging modality, and corresponding to a second point in time that follows, chronologically, the first point in time. Processing logic can identify a change in the position of one or more teeth and/or in the orientation of the one or more teeth. The change can correspond to a movement of the soft tissue. Processing logic can generate, based on the change, an updated 3D model representing a simulation of the movement of the soft tissue over time.

In some embodiments, processing logic can determine a secondary imaging modality, and can normalize at least a second subset of the records. Each record of the second subset can correspond to a subset of the plurality of points in time. Processing logic can generate a second timelapse visualization of the dental arch of the patient. The second timelapse visualization represents the normalized second subset of the plurality of records displayed in the chronological order of the subset of the plurality of points in time. Processing logic can provide, to the user device, the second timelapse visualization for presentation in the UI. In some embodiments, processing logic can cause the timelapse visualization and the second timelapse visualization to be presented concurrently in the user interface. In some embodiments, at least one point in time of the plurality of points in time of the timelapse visualization matches at least a second point in time of the subset of the plurality of points in time of the second timelapse visualization.

In some embodiments, a user can switch between imaging modalities during the presentation of the timelapse visualization. In some embodiments, to determine the secondary imaging modality, the processing logic can receive a user interaction identifying the secondary imaging modality. For example, a user of the user device can select a different imaging modality, and the timelapse visualization can switch to the different imaging modality.

In some embodiments, the multimodal timelapse system can generate a second timelapse visualization in an imaging modality for which no data currently exists for the patient. For example, one imaging modality (e.g., x-rays) can provide a more accurate clinical finding, but another modality (e.g., photographic images) may be easier for a patient to visualize the progression of the clinical finding. Processing logic can identify a second imaging modality of the plurality of imaging modalities. The plurality of records does not comprise a record corresponding to the second imaging modality. Processing logic can generate, based on the timelapse visualization, a second timelapse visualization of the dental arch of the patient. The second timelapse visualization represents the timelapse visualization in the second imaging modality. Processing logic provides, to the user device, the second timelapse visualization for presentation in the UI.

In embodiments, processing logic can use one or more machine learning models in the generation of the multimodal timelapse visualization of the patient. One type of machine learning model that may be used is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, gingiva, enamel, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

In some embodiments, the training dataset can contain hundreds, thousands, tens of thousands, hundreds of thousands, or more images (e.g., scan data, video data, and/or additional patient data) may be provided. In some embodiments, the training dataset can include labeled 3D color models generated from intraoral scan data of the dentition of a patient and/or color 2D images. In some embodiments, some or all of the data may be labeled with segmentation information, oral condition information, clinical finding information, and/or other information.

In some embodiments, some or all of the image-based data in the training dataset can be processed by a segmenter that segments the image-based data input individual teeth, and that outputs segmentation information (e.g., stored in segmentation data 358 of FIG. 3). The segmenter may be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, mandible, teeth, the TMJ's cartilage disc, etc. The segmenter may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects. In one embodiment, a single mask or map includes segmentation information for all identified classes of features and/or objects. Some types of features are location-specific features and are represented in one or more masks.

In some embodiments, the segmenter performs one or more image processing and/or computer vision techniques or operations to extract segmentation information from images. Such image processing and/or computer vision techniques may or may not include the use trained machine learning models. Accordingly, in some embodiments, segmenter does not include a machine learning model.

In some embodiments, the training dataset, and optionally the segmentation information (e.g., segmentation data 358) can be used to train one or more machine learning models to indicate clinical findings, oral conditions, treatment plans, primary imaging modalities, and/or primary image sources corresponding to an imaging modality. Training may be performed by inputting one or more data points and optionally corresponding segmentation information into the machine learning model one at a time. The data that is input into the machine learning model may include a single layer or multiple layers. In some embodiments, a recurrent neural network (RNN) is used. In such an embodiment, a second layer may include a previous output of the machine learning model (which resulted from processing a previous input).

The machine learning model processes the input to generate an output. An artificial neural network includes an input layer that consists of values in a data point. The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce.

Processing logic may then compare the generated values and/or categorizations to the known condition and/or label that was included in the training data item. Processing logic determines an error based on the differences between the output probability map and/or label(s) and the provided probability map and/or label(s). Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons,” where each layer receives input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the model. After one or more rounds of training, processing logic may determine whether a stopping criterion has been met. A stopping criterion may be a target level of accuracy, a target number of processed data items from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset (and optionally segmentation information) may be used to test the model. Testing the model can include performing unit tests, regression tests, and/or integration tests.

In some embodiments, processing logic can identify a second subset of the plurality of records that corresponds to points in time after completion of orthodontic treatment for the patient. For example, the second subset can include records captured during the retention phase of orthodontic treatment when patients are expected to wear retainers to maintain tooth positions achieved during active treatment. Processing logic can analyze the second subset to detect tooth movement patterns that indicate orthodontic relapse has occurred. Processing logic can compare current tooth positions from the second subset against the final tooth positions achieved at the end of orthodontic treatment to quantify the amount of relapse movement. In some embodiments, processing logic can perform comparative analysis by registering sequential records within the second subset to identify spatial displacement of individual teeth from their established post-treatment positions. The analysis can involve measuring changes in tooth coordinates, angulation, and/or inter-tooth spacing to quantify movement patterns that deviate from the stable positions achieved at orthodontic treatment completion, in embodiments.

In some embodiments, processing logic can determine the amount of orthodontic relapse by measuring positional changes in individual teeth or groups of teeth relative to their post-treatment positions. Processing logic can calculate displacement vectors, rotational changes, and spacing modifications that have occurred since treatment completion, in embodiments. Processing logic can update the timelapse visualization to display the measured relapse progression over time, e.g., using visual indicators such as color coding and/or measurement annotations.

In some embodiments, processing logic can identify compliance data associated with retainer wear patterns from sensor data and/or patient reporting systems. The compliance data can include one or more time periods of retainer non-wear. Time periods of retainer non-wear can refer to periods of time during which the patient failed to wear the retainer for the prescribed duration and/or frequency as specified in the orthodontic treatment protocol. For example, the time period can be characterized by wear times that fall below established compliance thresholds, such as wearing the retainer for less than the recommended 12-22 hours per day during initial retention phases or failing to maintain consistent nightly wear during long-term retention. In some embodiments, processing logic can determine periods of poor retainer compliance (e.g., periods of retainer non-wear) through sensor data 152 generated by electronic compliance indicators integrated within the retainer. The compliance data can include temperature sensors that detect when the retainer is in the patient's mouth, pressure sensors that measure occlusal contact, and/or accelerometers that track jaw movement patterns. Processing logic can analyze this sensor data to identify time periods when the retainer was not worn for the prescribed duration and/or when wear patterns deviate from expected compliance parameters. In some embodiments, processing logic can identify poor compliance periods (e.g., periods of retainer non-wear) by comparing actual wear data against predetermined compliance criteria stored in patient data 356. Processing logic can flag periods where daily wear time falls below threshold values, where there are extended gaps between wear sessions, and/or where the frequency of retainer use does not meet the prescribed schedule. In some embodiments, processing logic can correlate these identified non-compliance periods with subsequent changes in tooth positions detected through comparative analysis of sequential intraoral scans and/or photographs to establish temporal relationships between inadequate retainer use and orthodontic relapse progression.

In some embodiments, processing logic can correlate periods of poor retainer compliance with subsequent tooth movement to establish causal relationships between non-compliance and relapse progression. The processing logic can display visual indicators in the timelapse visualization that highlight temporal correlations between retainer non-wear periods and orthodontic relapse events. In some embodiments, the visual indicators can include color-coded timeline markers, overlay graphics, and/or annotation callouts that synchronize periods of documented non-compliance with corresponding tooth movement detected in the sequential records. Processing logic can use different visual emphasis techniques such as highlighting, outlining, and/or opacity changes to demonstrate the causal relationship between inadequate retainer wear and subsequent relapse progression.

In some embodiments, processing logic can generate predicted records representing prevention of orthodontic relapse through improved retainer compliance or intervention strategies. Processing logic can create predictive visualizations showing how consistent retainer wear can halt or reverse minor relapse movements. In some embodiments, the predictive visualizations can demonstrate specific scenarios where improved retainer compliance prevents tooth movement from the current tooth position. Processing logic can generate timelapse sequences showing stabilization of tooth positions when consistent retainer wear is maintained according to prescribed protocols. In some embodiments, processing logic can model different compliance improvement strategies such as increased daily wear time, more frequent retainer use, and/or transition to different retainer types. In some embodiments, processing logic can create comparative visualizations showing side-by-side outcomes between continued poor compliance and improved compliance scenarios. The predictive records can incorporate patient-specific factors such as age, bone density, and/or initial relapse severity to customize the prevention outcomes. Processing logic can generate motivational content that highlights the benefits of compliance improvement by showing how minor adjustments in retainer wear patterns can prevent the need for comprehensive orthodontic retreatment.

In some embodiments, the processing logic can determine whether the measured relapse amount satisfies a condition for correction through retainer realignment or other interventions. In some embodiments, processing logic can determine that the measured relapse amount satisfies the condition by comparing the quantified tooth movement to a threshold range that defines the limit of correctable relapse through retainer realignment treatment. For example, processing logic can evaluate factors such as the magnitude of individual tooth movement, the number of affected teeth, and/or the direction of relapse to determine whether the measured changes fall within parameters that can be effectively addressed through retainer-based correction rather than requiring comprehensive orthodontic retreatment. When the condition is satisfied, processing logic can generate predicted records showing potential relapse correction outcomes and update the timelapse visualization to include these corrective scenarios. For example, the predicted records can demonstrate the expected tooth movement patterns and timeline for achieving correction through retainer realignment treatment, showing patients the gradual return to their post-orthodontic treatment positions. Processing logic can generate multiple corrective scenarios corresponding to different retainer types or treatment protocols, enabling practitioners to present patients with various correction options and their associated timelines for achieving stable tooth positions.

In some embodiments, processing logic can provide adjustable progression rates for a clinical finding, allowing acceleration or deceleration of deterioration based on factors such as diet and/or behavioral habits. Processing logic can incorporate pateitn-specific risk factors and/or lifestyle information to modify the predicted rate of disease progression in future state visualizations. For example, for patients with high sugar diets or poor oral hygiene habits, processing logic can accelerate the predicted progression of caries or periodontal disease. As another example, for patients who implement behavioral modifications or dietary changes, processing logic can decelerate the predicted progression rates to reflect the positive impact of these changes.

In some embodiments, processing logic can create virtual patient representations for certain clinical findings (such as temporomandibular joint disorder), where the clinical finding is associated with a probability of causing another issue. For example, the clinical finding identification engine 320 can analyze malocclusion patterns and predict their likelihood of contributing to TMJ symptoms, muscle tension, and/or joint dysfunction. Processing logic can incorporate biomechanical principles and statistical relationship derived from clinical databases to quantify the probability that specific occlusion conditions will lead to secondary complications over time.

In some embodiments, processing logic can incorporate virtual articulator functionality to simulate jaw movements and/or predict forces on teeth for conditions such as root dehiscence and fenestrations. Processing logic can recreate the movements of the temporomandibular joints virtually, and analyze how occlusal forces are distrusted across the dental arch during various jaw movements, including protrusion, retrusion, lateral excursions, and/or centric relation to centric occlusion movements. The virtual articulator simulation may predict areas of excessive force concentration that may lead to bone loss around tooth roots or contribute to tooth fractures over time.

In some embodiments, processing logic can simulate, using at least one of a virtual articulator or occlusogram data of the patient, one or more forces acting on one or more teeth of the dental arch of the patient, wherein the one or more forces comprises parafunctional forces. Processing logic can generate one or more predicted records representing the dental arch of the patient affected by the one or more forces, wherein the one or more predicted records comprises at least one of a profile view image representing a facial change of the patient. Processing logic can update the timelapse visualization to include the one or more predicted records.

In some embodiments, processing logic can generate a virtual articulator model based on at least one record of the plurality of records. For example, the virtual articulator model can be constructed using three-dimensional dental arch data obtained from intraoral scans, CBCT data, and/or anatomical measurements extracted from the patient records. In some embodiments, the processing logic can process the plurality of records to identify jaw geometry, temporomandibular joint parameters, and/or condylar guidance angles specific to the patient. The virtual articulator model can incorporate patient-specific anatomical features including condylar inclination, Bennett angle, and/or incisal guidance to accurately simulate dental occlusion dynamics.

In some embodiments, processing logic can receive occlusogram data corresponding to occlusal contact patterns of the dental arch of the patient. The occlusogram data can be captured using pressure-sensitive films, digital bite analysis systems, and/or force measurement sensors integrated within the oral state capture system 110. The occlusogram data can indicate the location, intensity, and/or timing of tooth contacts during various jaw positions and movements. The processing logic can process the occlusogram data to identify premature contacts, occlusal interferences, and/or force distribution patterns across the dental arch.

In some embodiments, the processing logic can simulate forces acting on one or more teeth of the dental arch using the virtual articulator model and/or the occlusogram data. The force simulation can include normal masticatory forces as well as parafunctional forces such as those generated during bruxism, clenching, or grinding activities. The processing logic can apply biomechanical principles to calculate force vectors, moments, and/or stress distributions on individual teeth and supporting structures. In some embodiments, the force simulation can incorporate patient-specific factors such as muscle strength, jaw morphology, and/or occlusal relationships to provide accurate force predictions.

In some embodiments, the processing logic can predict potential root dehiscences and/or fenestrations affecting the one or more teeth based on the simulated forces. For example, the prediction algorithms can analyze force magnitude, direction, and/or duration to identify teeth at risk for bone loss around the root surfaces. The processing logic can use machine learning models trained on historical data correlating force patterns with bone defect development. The processing logic system can consider factors such as root morphology, bone density, and/or periodontal health status when generating predictions of root dehiscences and/or fenestrations.

In some embodiments, the processing logic can capture jaw movement data corresponding to protrusion, retrusion, lateral excursive movements, and/or centric relation to centric occlusion movements of the patient. The jaw movement data can be captured using video recording systems, electromagnetic tracking devices, and/or optical motion capture technology integrated within the oral state capture system 110. The processing logic can process the movement data to extract kinematic parameters including velocity, acceleration, and/or range of motion for each movement type. For example, the processing logic can record movement patterns during functional activities such as chewing, speaking, and/or swallowing.

In some embodiments, the processing logic can project the jaw movement data onto the virtual articulator model to determine range of motion parameters. The projection process can involve mapping the captured movement trajectories onto the virtual articulator's mechanical constraints and/or anatomical limitations. The processing logic can calculate maximum opening capacity, lateral excursion distances, and/or protrusive movement ranges based on the patient's specific anatomy. The range of motion parameters can be used to identify movement restrictions, asymmetries, and/or abnormal patterns that may contribute to temporomandibular joint disorders or occlusal problems.

In some embodiments, the processing logic can generate profile view images showing facial changes over time based on the virtual articulator model and cone beam computed tomography data. The processing logic can combine skeletal information from CBCT scans with soft tissue predictions derived from the virtual articulator model to create profile visualizations. The processing logic can simulate how changes in tooth position, jaw relationships, and/or occlusal vertical dimension affect facial appearance and profile aesthetics. The profile view images can demonstrate the relationship between internal skeletal changes and external facial modifications over time.

In some embodiments, the processing logic can update the timelapse visualization to include the predicted root dehiscences and/or fenestrations and the profile view images showing facial changes over time. The updated visualization can integrate the force simulation results with the temporal progression of clinical findings to demonstrate potential complications and/or treatment outcomes. The processing logic can highlight areas of predicted bone loss using color coding, annotations, and/or overlay graphics within the timelapse sequence. In some embodiments, The UI controller can provide the updated visualization to a user device for presentation in clinical consultation and patient education contexts, enabling practitioners to demonstrate the biomechanical consequences of occlusal problems and/or the benefits of corrective treatments.

In some embodiments, processing logic can provide treatment versus no treatment comparisons through filtering capabilities that remove predicted effects of clinical findings when a patient agrees to treatment, for example. In some embodiments, UI controller 330 of FIG. 3 can present side-by-side visualizations showing the predicted progression of an oral health condition with and without proposed interventions. When a patient agrees to a treatment for a clinical finding, processing logic can, using the filtering capability, eliminate the predicted negative effects of the clinical finding while maintaining the effect(s) of untreated clinical finding(s), providing a realistic assessment of expected outcomes following partial treatment acceptance.

In some embodiments, processing logic can incorporate a milestone and/or an event on a timeline corresponding to the timelapse visualization. Milestone and/or event incorporation on the timeline can include treatment planned items, completed work, failed work, and/or treatment progress for navigation purposes. In some embodiments, the timelapse data store 344 can maintain records of significant events in a patient's treatment history, allowing practitioners to correlate changes in oral health conditions with specific interventions or milestones. In some embodiments, the timeline navigation can enable a user (e.g., a practitioner) to jump to specific points in time where treatments were performed or complications occurred, facilitating analysis of treatment effectiveness and/or long-term outcomes.

In some embodiments, processing logic can identify, based on the plurality of records associated with the dental arch of the patient, one or more events indicative of a health status of the patient, wherein each of the one or more events corresponds a particular point in time of the plurality of points in time. An event can include an milestone, in embodiments. Processing logic can display, in the timelapse visualization, a visual indicator of at least one of the one or more events, wherein the visual indicator is displayed at the particular point in time corresponding to the at least one of the one or more events. In some embodiments, the visual indicator can be displayed for a particular time duration (e.g., 3 seconds, 30 seconds) starting at the particular point in time corresponding the event, or around the particular point in time corresponding to the event. In some embodiments, the one or more events can be displayed a list on the UI, near or within the timelapse visualization. In some embodiments, the one or more events can include predicted events. In some embodiments, events can include treatment planning milestones, completed work, failed work, treatment progress, and/or poor outcomes can be displayed and/or used to navigate patient records. In some embodiments, the events can include specific dental procedures, interventions, and/or therapeutic actions that have been identified and/or scheduled for a patient, e.g., based on an identified clinical finding.

In some embodiments, processing logic can provide social media sharing capabilities for positive motivational videos and/or improvement visualizations. In some embodiments, the UI controller 330 can generate patient-friendly versions of timelapse visualizations that highlight positive changes and/or treatment successes, which a patient can share through social media platforms, email, and/or text messaging. The sharing functionality can include privacy controls and/or patient consent mechanisms to provide appropriate use of medical imagining data while enabling the patient to celebrate oral health improvements and potentially motivate others to seek dental care.

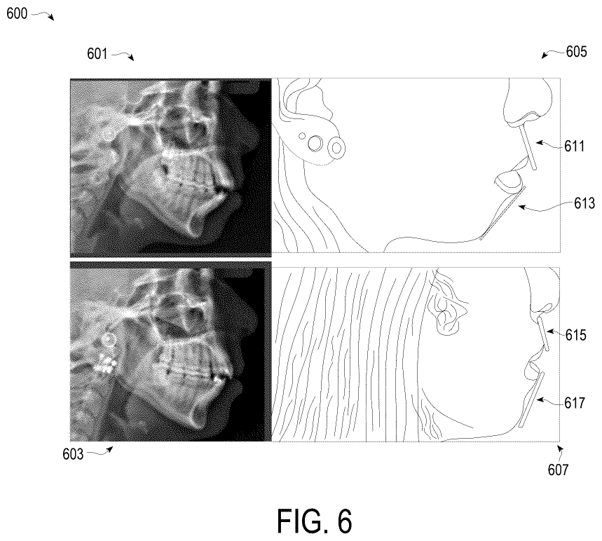

FIG. 6 illustrates an example of a multimodal presentation 600 of a visualization of a simulated outcome of orthodontic treatment generated by the multimodal timelapse system 215, in accordance with some embodiments of the present disclosure. In some embodiments, the multimodal timelapse system 215 can generate presentation 600 illustrating comprehensive treatment outcome visualizations that combine multiple imaging modalities. In some embodiments, the multimodal timelapse system 215 can produce both radiographic images and corresponding photographic simulations to demonstrate orthodontic treatment progress over time. For instance, in some embodiments, a radiograph alone may not provide the same visual impact as a photo simulation that shows soft tissue changes and facial profile improvements, and presenting both radiographic images and photographic simulations can help provide additional context for patient education and/or informed consent.

As illustrated in FIG. 6, radiographs 601 and 603 illustrate subsurface information about skeletal and dental structure changes, and profile photographs 605 and 607 provide surface information about facial appearance modifications. Lines 611 and 613 illustrate the relationship between internal anatomical changes and external facial profile improvements illustrated in photograph 605. Similarly, lines 615 and 617 illustrate the relationship between internal anatomical changes and external facial profile improvements illustrated in photograph 607. Thus, multimodal presentation 600 can enable visualization of both skeletal positioning changes and soft tissue appearance modifications over time.

As illustrated in FIG. 6, radiograph 601 displays a lateral x-ray view of the patient's skull, jaw, and dental structures at a first time point, and profile photograph 605 shows a photographic profile view of the patient's face at the corresponding first time point. Radiograph 603 displays a lateral x-ray view of the same patient at a second point in time, e.g., following orthodontic treatment. The first point in time can precede the second point in time. Profile photograph 607 shows a photographic profile view of the patient's face at the corresponding second point in time. In some embodiments, the second point in time can be a future point in time, and radiograph 603 and photograph 607 can be a visualization of a predictive simulation that displays the progression of orthodontic treatment over time.

In some embodiments, radiograph 601 and photograph 605 can represent radiographic data and photographic data, respectively, of the patient collected at the beginning of an orthodontic treatment plan. The multimodal timelapse system 215 can generate the radiograph 603 by processing radiograph 601 (and optionally other scan data of the patient) through predictive algorithms that model soft tissue adaptation. The multimodal timelapse system 215 can generate the photograph 607 by processing photograph 605 (and optionally other scan data of the patient) through predictive algorithms that model soft tissue adaptation. Using the output of the predictive algorithms, the output of the multimodal timelapse system 215 can simulate changes in lip position, facial profile, and smile aesthetics resulting from tooth movement during orthodontic treatment.

In some embodiments, the multimodal timelapse system 215 (e.g., via the timelapse generator 325) can apply transformation parameters to project predicted hard tissue movements onto corresponding soft tissue changes. In some embodiments, the multimodal timelapse system 215 can generate the lateral cephalometric radiographs 607 by processing CBCT data or existing radiographic records (e.g., radiograph 601) through bone remodeling simulation algorithms. In some embodiments, the multimodal timelapse system 215 can model skeletal changes including jaw repositioning and tooth root movement that occur during orthodontic treatment. In some embodiments, the system can correlate radiographic changes with corresponding facial profile modifications to provide comprehensive treatment visualization.

In some embodiments, the multimodal timelapse system 215 can present the radiographic views 601, 605 and photographic views 603, 607 simultaneously to demonstrate the relationship between internal skeletal changes and external facial appearance. In some embodiments, the multimodal timelapse system 215 can generate temporal sequences showing progressive changes from initial presentation through predicted treatment completion. In some embodiments, this integrated visualization approach can enhance patient understanding and treatment acceptance by showing both clinical and aesthetic outcomes.

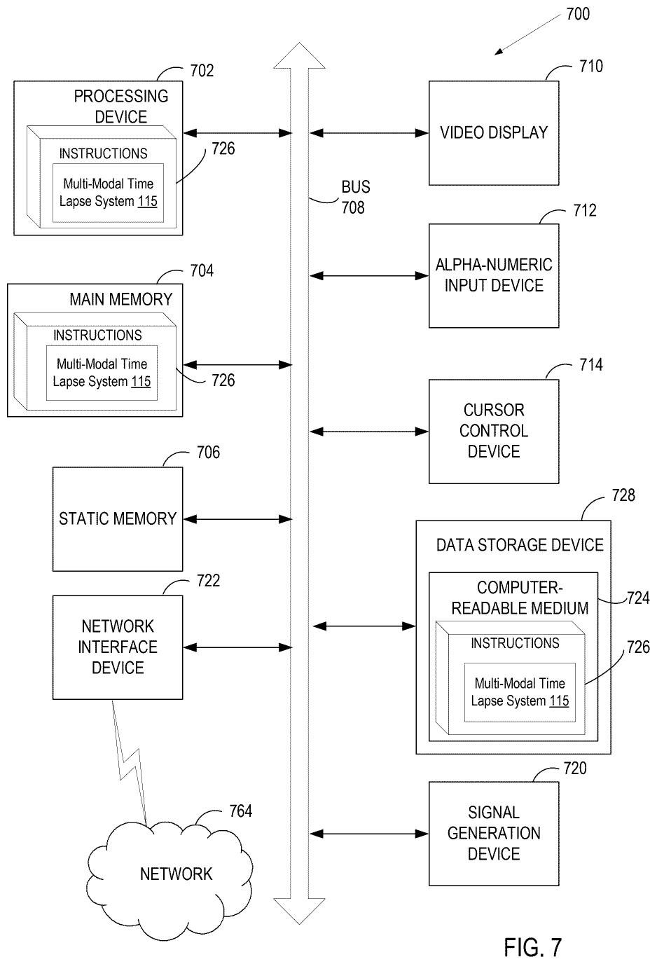

FIG. 7 illustrates a diagrammatic representation of a machine in the example form of a computing device 700 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computing device 700 corresponds to any computing device of FIG. 3.

The example computing device 700 includes a processing device 702 (e.g., a CPU), a main memory 704 (e.g., read-only memory (ROM), flash memory, dynamic random access memory (DRAM) such as synchronous DRAM (SDRAM), etc.), a static memory 706 (e.g., flash memory, static random access memory (SRAM), etc.), and a secondary memory (e.g., a data storage device 728), which communicate with each other via a bus 708.

Processing device 702 represents one or more general-purpose processors such as a microprocessor, central processing unit, or the like. More particularly, the processing device 702 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. In accordance with one or more aspects of the present disclosure, processing device 702 is configured to execute the processing logic (instructions 726, which may implement the multimodal timelapse system 215 of FIG. 3) for performing operations and steps discussed herein. While only a single example processing device is illustrated, the term “processing device” shall also be taken to include any collection of processing devices (e.g., computers) that individually or jointly execute a set (or multiple sets) of instructions to perform any one or more of the methods discussed herein.

The computing device 700 may further include a network interface device 722 for communicating with a network 764. The computing device 700 also may include a video display unit 710 (e.g., a liquid crystal display (LCD) or a cathode ray tube (CRT)), an alphanumeric input device 712 (e.g., a keyboard), a cursor control device 714 (e.g., a mouse, a touch-screen control device), and a signal generation device 720 (e.g., a speaker).

The data storage device 728 may include a machine-readable storage medium (or more specifically a non-transitory computer-readable storage medium) 724 on which is stored one or more sets of instructions 726 embodying any one or more of the methodologies or functions described herein. A non-transitory storage medium refers to a storage medium other than a carrier wave. The instructions 726 may also reside, completely or at least partially, within the main memory 704 and/or within the processing device 702 during execution thereof by the computer device 700, the main memory 704 and the processing device 702 also constituting computer-readable storage media.

The computer-readable storage medium 724 may also be used to store a multimodal timelapse system 215, which may correspond to the similarly named component of FIG. 3. The computer readable storage medium 724 may also store a software library containing methods for a multimodal timelapse system 215. While the computer-readable storage medium 724 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

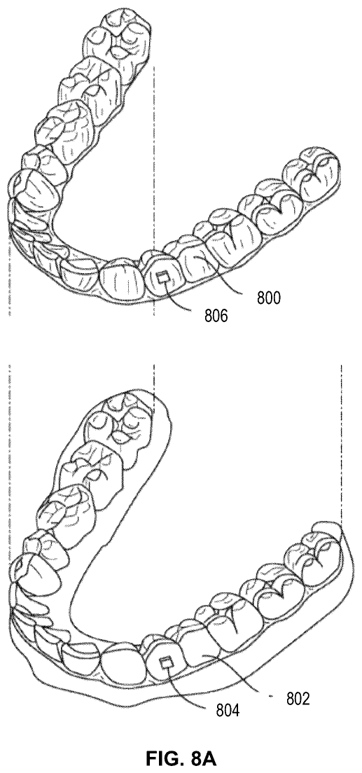

FIG. 8A illustrates an exemplary tooth repositioning dental appliance or aligner 800 that can be worn by a patient in order to achieve an incremental repositioning of individual teeth 802 in the jaw. The aligner 800 may be formed using modular trays and/or global plan data, as disclosed herein. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. A “polymeric material,” as used herein, may include any material formed from a polymer. A “polymer,” as used herein, may refer to a molecule composed of repeating structural units connected by covalent chemical bonds often characterized by a substantial number of repeating units (e.g., equal to or greater than 3 repeating units, optionally, in some embodiments equal to or greater than 10 repeating units, in some embodiments greater or equal to 30 repeating units) and a high molecular weight (e.g. greater than or equal to 10,000 Da, in some embodiments greater than or equal to 50,000 Da or greater than or equal to 100,000 Da).

Although polymeric aligners are discussed herein, the techniques disclosed may also be applied to aligners having different materials. Some embodiments are discussed herein with reference to orthodontic aligners (also referred to simply as aligners). However, embodiments also extend to other types of shells formed over molds, such as orthodontic retainers, orthodontic splints, sleep appliances for mouth insertion (e.g., for minimizing snoring, sleep apnea, etc.), palatal expanders and/or shells for non-dental applications. Accordingly, it should be understood that embodiments herein that refer to aligners also apply to other types of shells.

The aligner 800 can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some, most, or even all of the teeth will be repositioned at some point during treatment. Teeth that are moved can also serve as a base or anchor for holding the appliance as it is worn by the patient. Typically, no wires or other means will be provided for holding an appliance in place over the teeth. In some cases, however, it may be desirable or necessary to provide individual dental auxiliaries (e.g., dental attachments or other anchoring elements) 804 on teeth 802 with corresponding receptacles or apertures 806 in the aligner 800 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted dental auxiliaries suitable for use with orthodontic appliances are also described in patents and patent applications assigned to Align Technology, Inc., including, for example, U.S. Pat. Nos. 6,309,215 and 6,830,450.

FIG. 8B illustrates a tooth repositioning system 810 including a plurality of appliances 812, 814, 816. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 810 can include a first appliance 812 corresponding to an initial tooth arrangement, one or more intermediate appliances 814 corresponding to one or more intermediate arrangements, and a final appliance 816 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 812, 814, 816 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

In an example of indirect fabrication, a mold of a patient's dental arch may be fabricated from a digital model of the dental arch, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 812, 814, 816 after the digital models of the appliances 812, 814, 816 have been processed by processing logic of a computing device, such as the computing device in FIG. 7. The processing logic may include hardware (e.g., circuitry, dedicated logic, programmable logic, microcode, etc.), software (e.g., instructions executed by a processing device), firmware, or a combination thereof.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a 3D virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner and/or treatment planning software may determine a desired treatment outcome, which includes final positions and orientations for the patient's teeth. Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. The shape of each virtual 3D model will be different. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a rapid prototyping machine. The rapid prototyping machine then manufactures the mold using the digital model. One example of a rapid prototyping manufacturing machine is a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 812, 814, 816 can be pressure formed or thermoformed over the molds. Each mold may be used to fabricate an appliance that will apply forces to the patient's teeth at a particular stage of the orthodontic treatment. The appliances 812, 814, 816 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

After an appliance is formed over a mold for a treatment stage, the appliance is removed from the mold (e.g., automated removal of the appliance from the mold), and the appliance is subsequently trimmed along a cutline (also referred to as a trim line). The processing logic may determine a cutline for the appliance. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

In some embodiments, the dental appliances (e.g., orthodontic appliances) herein (or portions thereof) can be produced using direct fabrication, such as additive manufacturing techniques (also referred to herein as “3D printing) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances 812, 814, and 816. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliances 812, 814, and 816 can be directly fabricated using selective laser sintering. In some embodiments, selective laser sintering involves using a laser beam to selectively melt and fuse a layer of powdered material according to a desired cross-sectional shape in order to build up the object geometry. As yet another example, the appliances 812, 814, and 816 can be directly fabricated by fused deposition modeling. In some embodiments, fused deposition modeling involves melting and selectively depositing a thin filament of thermoplastic polymer in a layer-by-layer manner in order to form an object. In yet another example, material jetting can be used to directly fabricate the appliances 812, 814, and 816. In some embodiments, material jetting involves jetting or extruding one or more materials onto a build surface in order to form successive layers of the object geometry.

FIG. 8C illustrates a method 850 of orthodontic treatment using a plurality of appliances, in accordance with embodiments. One or more of the plurality of appliances may be formed using modular trays and/or global plan data, as disclosed herein. The method 850 can be practiced using any of the appliances or appliance sets described herein. In block 860, a first orthodontic appliance is applied to a patient's teeth in order to reposition the teeth from a first tooth arrangement to a second tooth arrangement. In block 870, a second orthodontic appliance is applied to the patient's teeth in order to reposition the teeth from the second tooth arrangement to a third tooth arrangement. The method 850 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the subsequent appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

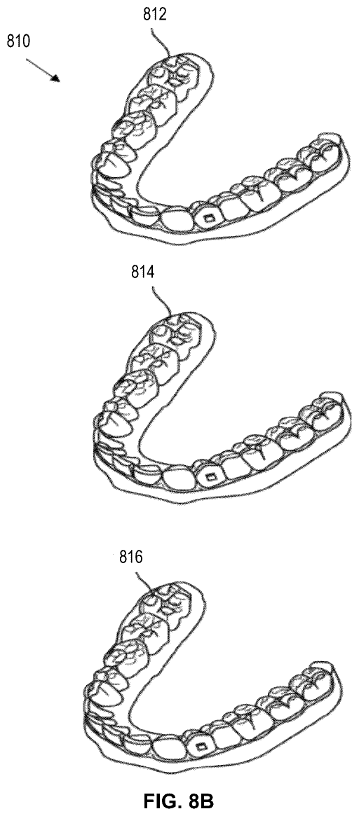

FIG. 9 illustrates a method 900 for designing an orthodontic appliance to be produced by direct fabrication, in accordance with embodiments. Some or all of the blocks of the method 900 can be performed by any suitable data processing system or device, e.g., one or more processors configured with suitable instructions. In some embodiments, method 900 is performed using treatment planning system 220 of FIG. 2.

In block 910, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In block 920, a force system to produce movement of the one or more teeth along the movement path may be determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In block 930, appliance design for an orthodontic appliance configured to produce the force system may be determined. Determination of the orthodontic appliance, appliance geometry, material composition, and/or properties can be performed using a treatment or force application simulation environment and/or application of machine learning. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA(Abaqus) software products from Dassault Systèmes of Waltham, MA.

Optionally, one or more orthodontic appliances can be selected for testing or force modeling. As noted above, a desired tooth movement, as well as a force system required or desired for eliciting the desired tooth movement, can be identified. Using the simulation environment, a candidate orthodontic appliance can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In block 940, instructions for fabrication of the orthodontic appliance incorporating the appliance design are generated. The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming. In some embodiments, the instructions for fabrication of the orthodontic appliance include instructions for performing modular trays and/or global plan data, as disclosed herein.

Method 900 may comprise additional blocks: 1) The upper arch and palate of the patient is scanned intraorally to generate three dimensional data of the palate and upper arch; and/or 2) The three dimensional shape profile of the appliance is determined to provide a gap and teeth engagement structures.

Although the above blocks show a method 900 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 900 may be performed with any suitable fabrication system or device, such as the embodiments described herein. Some of the blocks may be optional, and the order of the blocks can be varied as desired.

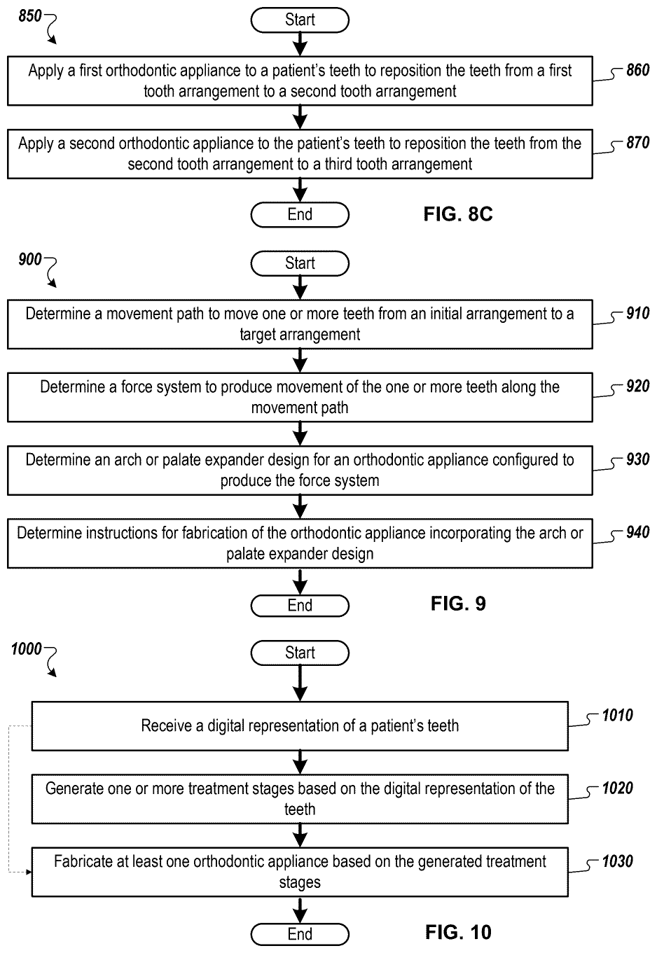

FIG. 10 illustrates a method 1000 for digitally planning an orthodontic treatment and/or design or fabrication of an appliance, in accordance with embodiments. The method 1000 can be applied to any of the treatment procedures described herein and can be performed by any suitable data processing system.

In block 1010, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 1020, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 1030, at least one orthodontic appliance is fabricated based on the generated treatment stages. For example, a set of appliances can be fabricated, each shaped according a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired. The fabrication of the appliance may include modular trays and/or global plan data, as disclosed herein.

In some instances, staging of various arrangements or treatment stages may not be necessary for design and/or fabrication of an appliance. As illustrated by the dashed line in FIG. 10, design and/or fabrication of an orthodontic appliance, and perhaps a particular orthodontic treatment, may include use of a representation of the patient's teeth (e.g., receive a digital representation of the patient's teeth at block 1010), followed by design and/or fabrication of an orthodontic appliance based on a representation of the patient's teeth in the arrangement represented by the received representation.

Some examples have been described with reference to orthodontic treatment plans that include a series of stages that are each associated with a different orthodontic aligner. It should be understood that any such examples described with reference to orthodontic treatment and a series of orthodontic aligners also applies to palatal expansion treatment and a series of palatal expanders. For palatal expansion treatment, a similar process may be performed as described above for orthodontic treatment. For example, an upper and/or lower dental arch and upper palate may be scanned using an intraoral scanner to generate a 3D model of the dental arch(es) and of the upper palate. A final shape (e.g., width) of the upper palate may be determined, and a series of treatment stages to progress from a current upper palate shape and a final target upper palate shape may be determined. For each treatment stage, a polymeric palatal expander may be fabricated, either via direct fabrication (e.g., direct 3D printing) or by 3D printing of a mold and thermoforming a palatal expander over the mold. Materials used for palatal expanders may be the same as or different from those used for orthodontic aligners in embodiments.

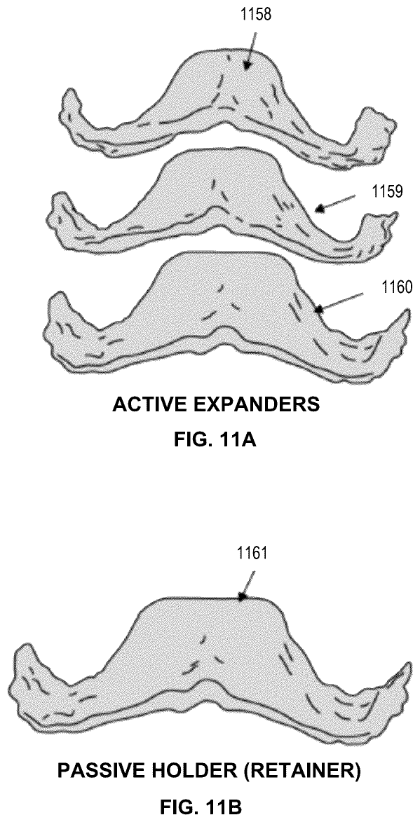

FIG. 11A shows an example of a series of palatal expanders that get progressively broader. For example, an initial upper palatal expander 1158 may have narrower palatal region than the intermediate palatal expander 1159 and a final palatal expander 1160. FIG. 11B illustrates an example of a passive holder (e.g., retainer) 1161 that may be worn after the series has completed expanding the patient's palate. In this example, the palatal expander retainer 1161 is similar or identical to the last of the palatal expanders in the sequence, although it may have a different configuration.

As mentioned above, a palatal expander as described herein can be one of a series of palatal expanders (incremental palatal expanders) that may be used to expand a subject's palate from an initial size/shape toward a target size/shape. For example, the methods and improvements described herein may be incorporated into a palatal expander or series of palatal expander as described, for example, in US20190314119A1, herein incorporated by reference in its entirety. A series of palatal expanders may be configured to expand the patient's palate by a predetermined distance (e.g., the distance between the molar regions of one expander may differ from the distance between the molar regions of the prior expander by not more than 2 mm, by between 0.1 and 2 mm, by between 0.25 and 1 mm, etc.) and/or by a predetermined force (e.g., limiting the force applied to less than 180 Newtons (N), to between 8-200 N, between 8-90 N, between 8-80 N, between 8-70 N, between 8-60 N, between 8-50 N, between 8-40 N, between 8-30 N, between 30-60 N, between 30-70 N, between 40-60 N, between 40-70 N, between 60-200 N, between 120-180 N, between 120-160 N, etc., including any range there between).

The palatal region may be between about 1-5 mm thick (e.g., between 1.5 to 3 mm, between 2 and 2.5 mm thick, etc.). The occlusal side may have a thickness of between about 0.5-2 mm (e.g., between 0.5 to 1.75 mm, between 0.75 to 1.7 mm, etc.). The buccal side may have a thickness of between about 0.25-1 mm (e.g., between 0.35 and 0.85 mm, between about 0.4 and 0.8 mm, etc.).

The dental devices described herein can include any of a number of features to facilitate the expansion process, improve patient comfort, and/or aid in insertion/retention of the dental devices in the patient's dentition. Examples of some features of dental devices are described in U.S. Patent Application Publication No. 2018/0153648A1, filed on Dec. 4, 2017, which is incorporated herein by reference in its entirety. For example, any of the dental devices described herein may include any number of attachment features that are configured to couple with corresponding attachments bonded to the patient's teeth. The dental devices may have regions of varying thickness. In any of the dental devices described herein can have varied thicknesses. For example, the thickness of a palatal region may be thicker or thinner than the thickness of tooth engagement regions. The palatal region of any of the palatal expanders may include one or more cut-out regions, which may enhance comfort and/or prevent problems with speech.

Any of the methods (including user interfaces) described herein may be implemented as software, hardware or firmware, and may be described as a non-transitory machine-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, computer models (e.g., for additive manufacturing) and instructions related to forming a dental device may be stored on a non-transitory machine-readable storage medium.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but may be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware, or code set forth above may be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the foregoing specification, a detailed description has been given with reference to specific exemplary embodiments. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but may refer to different and distinct embodiments, as well as potentially the same embodiment.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media, and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that may include one or more digital processing devices and memory to store executable instructions to perform the operations described in this specification.

While this specification contains many specific embodiment details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A system comprising:
a memory; and
a processing device to execute instructions from the memory to:
receive a plurality of records associated with a dental arch of a patient, wherein each record of the plurality of records corresponds to a point in time of a plurality of points in time and to an imaging modality of a plurality of imaging modalities;
provide the plurality of records for further processing, wherein the further processing comprises:
determining, based on processing of at least one record of the plurality of records, a clinical finding for the patient;
determining a primary imaging modality of the plurality of the imaging modalities based at least in part on the clinical finding;
normalizing at least a subset of the plurality of records; and
generating a timelapse visualization of the dental arch of the patient, wherein the timelapse visualization represents the normalized subset of the plurality of records displayed in chronological order of the plurality of points in time;
receive the timelapse visualization of the dental arch of the patient; and output the timelapse visualization for presentation to a display.

2. (canceled)

3. The system of claim 1, wherein the subset of the plurality of records comprises a first record corresponding to a first point in time of the plurality of points in time and to a first imaging modality of the plurality of imaging modalities, wherein the subset of the plurality of records comprises a second record corresponding to a second point in time of the plurality of points in time and to a second imaging modality of the plurality of imaging modalities, and wherein the timelapse visualization represents at least one of the first imaging modality or the second imaging modality.

4. (canceled)

5. (canceled)

6. The system of claim 1, wherein the further processing further comprise:
identifying a treatment corresponding to the clinical finding;
generating one or more predicted records representing the dental arch of the patient affected by the treatment at a future point in time; and
updating the timelapse visualization of the dental arch of the patient to include the one or more predicted records; and
wherein the instructions are further to:
receive the updated timelapse visualization; and
output the updated timelapse visualization for presentation to the display.

7. The system of claim 6, wherein the treatment comprises at least one of orthodontic treatment, retainer realignment treatment, restorative treatment, periodontal treatment, behavioral modification, appliance-based treatment, or interdisciplinary treatment.

8. The system of claim 6, wherein the further processing further comprises:
identifying one or more photographs of the patient, wherein the photographs comprise at least a mouth of the patient;
generating one or more predicted photographs of the patient, wherein the one or more predicted photographs represent an improvement of the at least the mouth of the patient affected by the treatment at a corresponding future point in time; and
generating, based on the one or more predicted photographs of the patient, a video representing the improvements of the at least the mouth of the patient over time; and
wherein the instructions are further to:
receive the video representing the improvements of the at least the mouth of the patient over time; and
output the video representing the improvements of the at least the mouth of the patient over time for presentation to the display.

9. (canceled)

10. The system of claim 6, wherein generating the one or more predicted records comprises:
determining an effect of the treatment on a condition of at least a portion of the dental arch of the patient at the future point in time; and
applying the effect to a most recent record of the plurality of records.

11. (canceled)

12. The system of claim 1, wherein the further processing further comprises:
generating one or more predicted records representing the dental arch of the patient affected by the clinical finding at a future point in time; and
updating the timelapse visualization of the dental arch of the patient to include the one or more predicted records; and
wherein the instructions are further to:
receive the updated timelapse visualization of the dental arch of the patient; and
output the updated timelapse visualization for presentation to the display.

13. (canceled)

14. (canceled)

15. (canceled)

16. (canceled)

17. (canceled)

18. (canceled)

19. (canceled)

20. (canceled)

21. The system of claim 1, wherein determining the clinical finding for the patient comprises:
providing, as input, the at least one of the plurality of records to an artificial intelligence model trained to provide an indication of the clinical finding; and
receiving, as output from the artificial intelligence model, the indication of the clinical finding.

22. The system of claim 1, wherein the clinical finding comprises an indication of at least one of: caries, malocclusion, periodontal disease, tooth wear, gingival recession, tooth facture, bruxism, temporomandibular joint disorder, structural anomaly, orthodontic relapse, or restorative disorder.

23. (canceled)

24. The system of claim 1, wherein determining the clinical finding for the patient comprises:
providing, as input, one or more of the plurality of records to an artificial intelligence model trained to provide an indication of the clinical finding;
receiving, as output from the artificial intelligence model, a plurality of indications of the clinical finding, each indication corresponding to a record, wherein each indication comprises a confidence level; and
identifying the clinical finding with a highest confidence level.

25. (canceled)

26. The system of claim 1, wherein the primary imaging modality that corresponds to the clinical finding is determined based on a ranking of the plurality of imaging modalities.

27. The system of claim 1, wherein the plurality of imaging modalities comprise at least one of: intraoral scan, extraoral scan, near-infrared image, cone beam computed tomography (CBCT), photograph, video, or radiograph.

28. The system of claim 1, wherein the timelapse visualization is an animated representation of the normalized subset of the plurality of records.

29. The system of claim 1, wherein generating the timelapse visualization of the dental arch comprises:
generating, for one or more pairs of chronologically consecutive records in the subset of the plurality of records, one or more intermediate images; and
inserting, for each of the one or more pairs, the one or more intermediate images in between the corresponding pair of chronologically consecutive records.

30. (canceled)

31. The system of claim 1, wherein the timelapse visualization comprises data corresponding to at least one of the plurality of imaging modalities other than the primary imaging modality.

32. (canceled)

33. The system of claim 1, wherein the at least one record that is processed to determine the clinical finding has a first imaging modality, and wherein the primary imaging modality is a second imaging modality that is different from the first imaging modality.

34. The system of claim 1, wherein the further processing further comprises:
determining, based on the processing of the at least one record of the plurality of records, a plurality of indications of the clinical finding for the patient, wherein each indication of the clinical finding corresponds to an imaging modality of the plurality of imaging modalities;
determining a discrepancy between at least two indications of the clinical finding of the plurality of indications of the clinical finding;
ranking, based on a correlation between each of the at least two indications of clinical finding and the corresponding imaging modality, each of the at least two indications of the clinical finding; and
resolving the discrepancy based on the ranking.

35. (canceled)

36. The system of claim 1, wherein the further processing further comprises:
determining, based on the processing of the at least one record of the plurality of records, a plurality of clinical findings for the patient;
generating one or more predicted records representing the dental arch of the patient affected by the plurality of clinical findings at a future point in time; and
generating an updated timelapse visualization of the dental arch by updating the timelapse visualization to include the one or more predicted records; and
wherein the instructions are further to:
receive the updated timelapse visualization of the dental arch; and
output the updated timelapse visualization for presentation to the display.

37. The system of claim 36, wherein in response to receiving a user interaction indicating a first clinical finding of the plurality of clinical findings, the further processing further comprises:
determining a subset of clinical findings comprising the plurality of clinical findings other than first clinical finding;
generating a second set of one or more predicted records representing the dental arch of the patient affected by the subset of clinical findings at the future point in time; and
generating a second updated timelapse visualization of the dental arch by updating the timelapse visualization to include the second set of the one or more predicted records; and
wherein the instructions are further to:
receive the second updated timelapse visualization of the dental arch; and
output the second updated timelapse visualization for presentation to the display.

38. (canceled)

39. The system of claim 1, wherein the further processing further comprises:
generating a first set of one or more predicted records representing the dental arch of the patient affected by the clinical finding at a future point in time;
generating a second set of one or more predicted records representing the dental arch of the patient affected by behavior modifications corresponding to the clinical finding at the future point in time;
generating a third set of one or more predicted records representing the dental arch of the patient affected by a first treatment corresponding to the clinical finding at the future point in time;
generating a fourth set of one or more predicted records representing the dental arch of the patient affected by a second treatment corresponding to the clinical finding at the future point in time; and
generating a plurality of updated timelapse visualizations of the dental arch of the patient, wherein each updated timelapse visualization comprises the timelapse visualization updated to include one of the first set of one or more predicted records, the second set of one or more predicted records, the third set of one or more predicted records, or the fourth set of one or more predicted records; and
wherein the instructions are further to:
receive the plurality of updated timelapse visualizations; and
output the plurality of updated timelapse visualizations for presentation to the display.

40. The system of claim 1, further comprising:
identifying a first record of the plurality of records corresponding to a first imaging modality of the patient at a first point in time;
identifying a second record of the plurality of records corresponding to a second imaging modality of the dental arch of the patient at the first point in time;
identifying, based on at least one of the first record or the second record, a position of one or more teeth of the dental arch and an orientation of the one or more teeth of the dental arch; and
generating, based on the first record and the second record, a three-dimensional model of the dental arch of the patient, wherein the three-dimensional model comprises the one or more teeth of the dental arch and a jaw of the dental arch.

41. (canceled)

42. (canceled)

43. (canceled)

44. (canceled)

45. (canceled)

46. (canceled)

47. The system of claim 1, wherein the further processing further comprises:
identifying, based on the plurality of records associated with the dental arch of the patient, one or more events indicative of a health status of the patient, wherein each of the one or more events corresponds a particular point in time of the plurality of points in time; and
displaying, in the timelapse visualization, a visual indicator of at least one of the one or more events, wherein the visual indicator is displayed at the particular point in time corresponding to the at least one of the one or more events.

48. The system of claim 1, wherein the further processing further comprises:
simulating, using at least one of a virtual articulator or occlusogram data of the patient, one or more forces acting on one or more teeth of the dental arch of the patient, wherein the one or more forces comprises parafunctional forces;
generating one or more predicted records representing the dental arch of the patient affected by the one or more forces, wherein the one or more predicted records comprises at least one of a profile view image representing a facial change of the patient; and
updating the timelapse visualization to include the one or more predicted records.

49. The system of claim 1, wherein the further processing further comprises:
identifying a second subset of the plurality of records, wherein the second subset comprises one or more records of the plurality of records that have a corresponding point in time after a completion of an orthodontic treatment for the patient;
detecting, based on processing of the second subset of the plurality of records, tooth movement indicating orthodontic relapse;
determining an amount of the orthodontic relapse by comparing one or more tooth positions of the second subset of the plurality of records to one or more corresponding final tooth positions associated with the orthodontic treatment for the patient; and
updating the timelapse visualization to display the amount of orthodontic relapse.

50. The system of claim 49, wherein the further processing further comprises:
identifying compliance data associated with a retainer worn by the patient, wherein the compliance data comprises one or more time periods of retainer non-wear;
correlating the amount of the orthodontic relapse with the one or more time periods of retainer non-wear; and
displaying, in the timelapse visualization, one or more visual indicators correlating the orthodontic relapse with the one or more time periods of retainer non-wear.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. A non-transitory computer-readable storage medium comprising instructions that, when executed by a processing device, cause the processing device to:
receive a plurality of records associated with a dental arch of a patient, wherein each record of the plurality of records corresponds to a point in time of a plurality of points in time and to an imaging modality of a plurality of imaging modalities;
provide the plurality of records for further processing, wherein the further processing comprises:
determining, based on processing of at least one record of the plurality of records, a clinical finding for the patient;
determining a primary imaging modality of the plurality of the imaging modalities based at least in part on the clinical finding;
normalizing at least a subset of the plurality of records; and
generating a timelapse visualization of the dental arch of the patient, wherein the timelapse visualization represents the normalized subset of the plurality of records displayed in chronological order of the plurality of points in time;
receive the timelapse visualization of the dental arch of the patient; and
output the timelapse visualization for presentation to a display.

106.

107. The non-transitory computer-readable storage medium of claim 105, wherein the subset of the plurality of records comprises a first record corresponding to a first point in time of the plurality of points in time and to a first imaging modality of the plurality of imaging modalities, wherein the subset of the plurality of records comprises a second record corresponding to a second point in time of the plurality of points in time and to a second imaging modality of the plurality of imaging modalities, and wherein the timelapse visualization represents at least one of the first imaging modality or the second imaging modality.

108. (canceled)

109. (canceled)

110. The non-transitory computer-readable storage medium of claim 105, wherein the further processing comprises:
identifying a treatment corresponding to the clinical finding;
identifying one or more predicted records representing the dental arch of the patient affected by the treatment at a future point in time; and
updating the timelapse visualization of the dental arch of the patient to include the one or more predicted records; and
wherein the processing device is further to:
receive the timelapse visualization of the dental arch of the patient; and
output the updated timelapse visualization for presentation to the display.

111. The non-transitory computer-readable storage medium of claim 110, wherein the treatment comprises at least one of orthodontic treatment, retainer realignment treatment, restorative treatment, periodontal treatment, behavioral modification, appliance-based treatment, or interdisciplinary treatment.

112. The non-transitory computer-readable storage medium of claim 110, wherein the further processing further comprises:
identifying one or more photographs of the patient, wherein the photographs comprise at least a mouth of the patient;
generating one or more predicted photographs of the patient, wherein the one or more predicted photographs represent an improvement of the at least the mouth of the patient affected by the treatment at a corresponding future point in time; and
generating, based on the one or more predicted photographs of the patient, a video representing the improvements of the at least the mouth of the patient over time; and
wherein the processing device is further to:
receive the video representing the improvements of the at least the mouth of the patient over time; and
output the video representing the improvements of the at least the mouth of the patient over time for presentation to the display.

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)
